(19) United States
(12) Patent Application Publication
(10) Pub. No.: US 2008/0090770 A1
(43) Pub. Date: Apr. 17, 2008

(54) Modulation of Muc1 Mediated Signal Transduction

(76) Inventors: Michael P. Belmares, San Jose, CA (US); Peter S. Lu, Mountain View, CA (US); Jonathan David Garman, San Jose, CA (US); Albert A. Jecminek, San Antonio, TX (US); Surender Kharbanda, Natick, MA (US); Naoki Agata, Brookline, MA (US); Donald W. Kufe, Wellesley, MA (US)

Correspondence Address: FULBRIGHT & JAWORSKI L.L.P., 600 CONGRESS AVE., SUITE 2400, AUSTIN, TX, 78701, US

(21) Appl. No.: 10/553,028

(22) PCT Filed: Apr. 12, 2004
(86) PCT No.: PCT/US04/11195

§ 371 Date: Apr. 26, 2007

Related U.S. Application Data
(60) Provisional application No. 60/462,111, filed on Apr. 11, 2003.
(60) Provisional application No. 60/467,728.
(60) Provisional application No. 60/475,595, filed on Jun. 4, 2003.
(60) Provisional application No. 60/502,111, filed on Sep. 11, 2003.
(60) Provisional application No. 60/524,188, filed on Nov. 21, 2003.

(51) Int. Cl. A61K 38/08; A61K 38/06

(52) U.S. Cl. 514/17; 514/18

(57) ABSTRACT

The present invention provides compositions and methods for inhibiting the binding of the carboxy-terminus of MUC1 to PDZ domain(s) and to enhance the sensitivity of MUC1 expressing cancer cells to chemotherapeutic agents.

This application claims priority to U.S. Provisional Application Ser. No. 60/462,111, filed Apr. 11, 2003, U.S. Provisional Application Ser. No. 60/467,728, filed May, 2, 2003, U.S. Provisional Application Ser. No. 60/475,595, filed Jun. 4, 2003, U.S. Provisional Application Ser. No. 60/502,111, filed Sep. 11, 2003 and U.S. Provisional Application Ser. No. 60/524,188, filed Nov. 21, 2003, all herein incorporated by reference

### BACKGROUND OF THE INVENTION

The present invention relates generally to the field of cancer therapy and more specifically to the use of modulators or agents that interact with MUC1 as a point on intervention in cancer therapy.

The human MUC1 mucin glycoprotein is expressed on the apical borders of secretory epithelial cells on the luminal surface of most glandular epithelia (Kufe et al., 1984). In carcinomas, MUC1 is highly overexpressed throughout the entire cell membrane and cytoplasm (Kufe et al., 1984; Perey et al., 1992). As such, the aberrant pattern of MUC1 expression in carcinoma cells may confer a function for MUC1 normally found at the apical membrane to the entire cell membrane. The hallmark of MUC1 mucin is an ectodomain comprising a glycosylated 20 amino acid extracellular sequence that is tandemly repeated 25-100 times in each molecule (Strouss & Decker, 1992). The mucin glycosylation level appears to be lower in cancer cells than normal cells of ductal epithelial tissue (Kufe, U.S. Pat. No. 5,506,343). This hypoglycosylation results in the exposure of tumor-specific epitopes that are hidden in the fully glycosylated mucin.

Over ninety percent of breast cancers show an increased expression of MUC1 (also known as Mucin, Epithelial Membrane Antigen, Polymorphic Epithelial Mucin, Human Milk Fat Globule Membrane antigen, Episialin, DF-3, etc., see Barry & Sharkey, 1985). Several clinical studies have suggested that mucinous tumor antigens expressed on the cell surface of tumor cells associate with poor prognosis of a variety of cancer types (Itzkowitz et al., 1990).

MUC1 is expressed as both a transmembrane form and a secreted form (Finn et al., 1995). The repeating sialyl epitopes of MUC1 (the “ectodomain”) are shed into the serum (Reddish et al., 1996). The N-terminal ectodomain (the extracellular domain that is cleaved) of MUC1 consists of a variable number of the 20-amino acid tandem repeats that are subject to O-glycosylation. This mucin extends far above the cell surface and past the glycocalyx making it easily available for interactions with other cells. The C-terminal region of MUC1 includes a 37 amino acid transmembrane domain and a 72 amino acid cytoplasmic tail that contains sites for tyrosine phosphorylation. An approximately 45-amino acid extracellular domain remains following cleavage of the ectodomain. It is not known what enzyme is responsible for the cleavage of the ectodomain at this time.

The cytoplasmic domain of MUC1 (“MUC1/CD”) encompasses multiple sub-domains that are important in intracellular signaling in cancer cells. β-catenin binds directly to MUC1/CD at a SAGNGGSSL motif (Yamamoto et al., 1997). β-catenin, a component of the adherens junctions of mammalian epithelium, binds to cadherins at the intracellular surface of the plasma membrane and performs a signaling role in the cytoplasm as the penultimate downstream mediator of the wnt signaling pathway (Takeichi, 1990; Novak & Dedhar, 1999). The ultimate mediator of the wnt pathway is a nuclear complex of β-catenin and lymphoid enhancer factor/T cell factor (Lef/Tcf) that stimulates the transcription of a variety of target genes (see e.g., Molenaar et al., 1996; Brunner et al., 1997). Defects in the β-catenin-Lef/Tcf pathway are involved in the development of several types of cancers Novak & Dedhar, 1999).

Glycogen synthase kinase 3β (GSK3β) also binds directly to MUC1/CD and phosphorylates serine in a DRSPY site adjacent to the β-catenin binding motif, thereby decreasing the association between MUC1 and β-catenin (Li et al., 1998). In addition, the c-Src tyrosine kinase also binds to and phosphorylates a MUC1/CD SPYEKV motif, resulting in an increased interaction between MUC1/CD and β-catenin and a decreased interaction between MUC1/CD and GSK3β (Li et al., 2001).

MUC1 associates also constitutively with the epidermal growth factor receptor (EGF-R, HER1) at the cell membrane and activated EGF-R induces phosphorylation of the MUC1/CD SPYKEV motif (Li et al., 2001(a)). EGF-R mediated phosphorylation of MUC1/CD appears to increase the interaction of MUC1 with c-Src and β-catenin and downregulate the interaction between MUC1 and GSK3β. These results support a model wherein MUC1 integrates the signaling among c-Src, β-catenin and GSK3β pathways and dysregulation of this integrated signaling by aberrant overexpression of MUC1 in cancer cells could promote the transformed phenotype (Li et al., 2001(a)).

The Armadillo protein p120^{ctn} also binds directly to MUC1/CD resulting in the nuclear localization of p120 (Li & Kufe, 2001). p120 has been implicated in cell transformation and altered patterns of p120 expression have been observed in carcinomas (see e.g., Jawhari et al., 1999; Shimazui et al., 1996). p120 is a v-Src tyrosine kinase substrate, binds to E-cadherin, and is implicated as a transcriptional coactivator (Reynolds et al., 1989; Reynolds et al., 1994; Daniels & Reynolds, 1999). The observation that p120 localizes to both cell junctions and the nucleus, have supported a role for p120, like β-catenin, in the regulation of both cell adhesion and gene transcription. Decreased cell adhesion resulting from association of MUC1 and p120 may be involved in increased metastatic potential of MUC1-expressing tumor cells.

Thus, the available evidence indicates that MUC1/CD functions to transfer signals from the extracellular domain to the nucleus, and utilizes signaling mechanisms that have been implicated in adhesion receptor and growth factor signaling and cellular transformation. It is desirable to identify compositions and methods related to modulation of the MUC1-mediated signaling and its putative role in cellular transformation.

### SUMMARY OF THE INVENTION

The present invention provides methods for inhibiting the binding of the cytoplasmic domain of MUC1 to a PDZ domain, wherein the PDZ domain may suitably be ZO-1 d2, SIP1 d1, LIM MYSTIQUE, AIPC, KIAA0751, MAST2, PRIL-16 d1, GRIP2 d5, SITAC 18, NSP or KIAA1526 d1, and wherein the PDZ domain may be within a MUC1-expressing cancer; enhancing the sensitivity of MUC1-expressing cancer cells to chemotherapeutic agents comprising contacting the MUC1-expressing cancer cell with an effective amount of an agent that inhibits the binding of MUC1 to a PDZ domain; killing MUC1-expressing cancer cells comprising contacting the MUC1-expressing cancer cells with an effective amount of a chemotherapeutic agent and an agent that inhibits the binding of MUC1 to a PDZ domain; inhibiting the proliferation of MUC1-expressing cancer cells comprising contacting the MUC1-expressing cancer cells with an effective amount of an agent that inhibits the binding of MUC1 to a PDZ domain; treating a MUC1-expressing cancer by administering an effective amount of an agent that inhibits the binding of MUC1 to a PDZ domain; treating a MUC1-expressing cancer by administering an effective amount of an agent that inhibits the binding of MUC1 to a PDZ domain and an effective amount of a chemotherapeutic agent; and inhibiting the colocalization or association of MUC1 with one or more of the proteins FGFR, EGFR, ErbB2, ErbB3, ErbB4, β-catenin, γ-catenin, c-SRC or GSK3β.

Agents that inhibit the binding of MUC1 to a PDZ domain suitably include peptides of the formula X¹-aa²-aa¹-aa⁰, wherein aa⁰ is a hydrophobic aliphatic amino acid residue or a hydrophobic aromatic amino acid residue, aa² is a hydrophobic aliphatic amino acid residue, hydrophobic aromatic amino acid residue, polar amino acid residue, basic amino acid residue or an acidic amino acid residue, aa¹ is an amino acid residue and X¹ is a sequence of 0 to 50 amino acid residues. In some embodiments, aa⁰ is V, L, A, I, S or Y and aa² is V, L, A, I, F, Y, W, Q, N, S, T, R, K, D or E. In some embodiments, a²-aa¹-aa⁰ is a sequence selected from SEQ ID NO: 1 through SEQ ID NO: 40. In some embodiments, the carboxy-terminus of the peptide of formula X¹-aa²-aa¹-aa⁰ comprises the carboxy-terminal 4, 5 6, 7, 8 or 9 amino acid residues of a nine amino acid residue sequence selected from SEQ ID NO: 41 through SEQ ID NO: 94. In some embodiments, the carboxy-terminus of the peptide of formula X¹-aa²-aa¹-aa⁰ comprises the carboxy-terminal 4, 5 6, 7, 8, 9, 10, 11, 12, 13, 14, 15, 16, 17, 18, 19 or 20 amino acid residues of SEQ ID NO: 95 or SEQ ID NO: 96. In some embodiments, the amino-terminus of X¹ of the peptide X¹-aa²-aa⁰ comprises X²-X³, wherein X² is a transmembrane transporter peptide sequence and X³ is an optional linker sequence. In some embodiments, X² is a sequence selected from SEQ ID NO: 97 through SEQ ID NO: 127. In some embodiments, X² is SEQ ID NO: 102, SEQ ID NO: 108 or SEQ ID NO: 119.

In embodiments that encompass a cancer cell, the cancer cell may be a breast cancer cell, an ovarian cancer cell, a lung cancer cell, a pancreatic cancer cell, a prostate cancer cell, a stomach cancer cell, a small intestine cancer cell, a colon cancer cell, a liver cancer cell, a kidney cancer cell, an esophageal cancer cell, a head and neck cancer cell, a testicular cancer cell, a blood cancer cell, a bone marrow cancer cell, or a cancer cell of another tissue. In some embodiments, the cancer cell is within a patient.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1: 293 cells were transected to express pIRESpuro2-Flag-MUC1-CD(1-72) or pIRESpuro2-Flag-MUC1-CD(1-68). Lysates were subjected to immunoprecipitation with anti-FGFR3 or IgG as a control. The immunoprecipitates and lysate not subjected to immunoprecipitation were analyzed by immunoblotting with anti-MUC1-CD.

FIG. 2: 293 cells were transected to express pIRESpuro2-Flag-MUC1-CD(1-72) or pIRESpuro2-Flag-MUC1-CD(1-68). Lysates were subjected to immunoprecipitation with anti-EGFR or IgG as a control. The immunoprecipitates and lysate not subjected to immunoprecipitation were analyzed by immunoblotting with anti-MUC1-CD.

FIG. 3: Profile of the binding of 0.01 μM C-terminus of MUC1 to PDZ domains.

FIG. 4: Profile of the binding of 0.1 μM C-terminus of MUC1 to PDZ domains.

FIG. 5: Summary of effects of the knockdown of Lim Mystique (LIM-M) or KIAA0751, aka RIM2 (KIAA) on CDDP-induced apoptosis in A549 and HCT116/MUC1 cells. At 48 hr after transfection of siRNAs specific for Lim Mystique or KIAAO751, cells were treated with or without 100 μM CDDP for 24 hr and then analyzed for apoptosis.

FIG. 6: Summary of effects of the knockdown of KIAAO751, aka RIM2 (KIAA) on CDDP-induced apoptosis in HCT116/Vector cells. At 48 hr after transfection of siRNA specific for KIAA0751, cells were treated with 0, 10 and 100 μM CDDP for 24 hr and then analyzed for apoptosis.

FIG. 7: Summary of effects of the knockdown of KIAAO751, aka RIM2 (KIAA) or ZO-1 on CDDP-induced apoptosis in HCT116/MUC1 cells. At 48 hr after transfection of siRNA specific for KIAA0751 or ZO-1 SIP1, cells were treated with or without 100 μM CDDP for 24 hr and 48 hr and then analyzed for apoptosis.

FIG. 8: Summary of effects of the knockdown of SIP1 on CDDP-induced apoptosis in A549 or HCT116/MUC1 cells. At 48 hr after transfection of siRNA specific for SIP1, cells were treated with or without 100 μM CDDP for 24 hr and 48 hr and then analyzed for apoptosis.

FIG. 9: Summary of results of titration of RIM2 (KIAAO751) and ZO1 d2 with two biotinylated carboxy-terminal MUC1 isotypes, i.e., with an A/T substitution at the fifth amino acid residue from the carboxy-terminus (AAA and AAT). Results indicate similar binding affinities for both ZO1 d2 and RIM2.

FIG. 10: Summary of results of competitive inhibition of selected peptides of the binding of biotinylated TAT-MUC1 to RIM2

FIG. 11: Summary of results of screening the binding of 0.01 μM biotinylated SEQ ID NO: 137 to PDZ domains.

FIG. 12: Summary of results of screening the binding of 0.025 μM biotinylated SEQ ID NO: 136 to PDZ domains.

FIG. 13: Summary of results of screening the binding of 0.05 μM biotinylated SEQ ID NO: 138 to PDZ domains.

### DETAILED DESCRIPTION OF THE INVENTION

### I. PDZ Domains and Related Ligands

PDZ domains are modular protein interaction domains that play a cellular role in protein targeting and protein complex assembly. These domains are relatively small (≧90 residues), fold into a compact globular structure and generally have N- and C-termini that are close to one another in the folded structure. Thus the domains are highly modular and could easily have been integrated into existing proteins without significant structural disruption through the course of evolution. PDZ domains typically consists of six β-strands (βA-βF) and two α-helices (αA and αB). Peptide ligands bind in an extended groove between strand βB and helix αB by a mechanism referred to as β-strand addition, wherein the peptide serves as an extra β-strand that is added onto the edge of a pre-existing β-sheet within the PDZ domain (Harrison, 1996).

PDZ domains recognize specific C-terminal sequence motifs that are usually about four to five residues in length (Niethammer et al., 1998). One nomenclature utilized for residues within the PDZ-binding motif refers to the C-terminal residue as the P₀ residue and subsequent residues towards the N-terminus are termed P₋₁, P₋₂, P₋₃, etc. Extensive peptide library screens suggest that the P₀ and P₋₂ residues are most critical for recognition (Songyang et al., 1997; Schultz et al., 1998). These studies also show that PDZ domains can be divided into at least three main classes on the basis of their preferences for residues at these two sites: class I PDZ domains recognize the motif S/T-X-Φ-COOH (where Φ is a hydrophobic amino acid and X is any amino acid); class II PDZ domains recognize the motif Φ-X-Φ-COOH; and class m PDZ domains recognize the motif X—X—C—COOH. There are a few other PDZ domains that do not fall into any of these specific classes.

The four terminal amino acids of the cytoplasmic domain of MUC1 are serine, alanine aspargine and leucine. Both leucine and alanine are hydrophobic amino acids, albeit that alanine is significantly less hydrophobic than leucine. This carboxy-terminal region of MUC1 is highly conserved over a number of species suggesting that this sequence is directed towards some cellular functionality. The present invention identifies the MUC1 carboxy-terminus as a ligand for select PDZ domains.

### II. Peptides

A “fusion protein” or “fusion polypeptide” as used herein refers to a composite protein, i.e., a single contiguous amino acid sequence, made up of two (or more) distinct, heterologous polypeptides that are not normally fused together in a single amino acid sequence. Thus, a fusion protein can include a single amino acid sequence that contains two entirely distinct amino acid sequences or two similar or identical polypeptide sequences, provided that these sequences are not normally found together in the same configuration in a single amino acid sequence found in nature. Fusion proteins can generally be prepared using either recombinant nucleic acid methods, i.e., as a result of transcription and translation of a recombinant gene fusion product, which fusion comprises a segment encoding a polypeptide of the invention and a segment encoding a heterologous protein, or by chemical synthesis methods well known in the art.

As used herein, the term “PDZ domain” refers to protein sequence (i.e., modular protein domain) of less than approximately 90 amino acids (i.e., about 80-90, about 70-80, about 60-70 or about 50-60 amino acids), characterized by homology to the brain synaptic protein PSD-95, the Drosophila septate junction protein Discs-Large (DLG), and the epithelial tight junction protein ZO1 (ZO1). PDZ domains are also known as Discs-Large homology repeats (“DHRs”) and GLGF repeats. PDZ domains generally appear to maintain a core consensus sequence (Doyle, 1996).

Exemplary PDZ domain-containing proteins and PDZ domain sequences are shown in Table 3 in Example 6. The term “PDZ domain” also encompasses variants (e.g., naturally-occurring variants) of the sequences (e.g., polymorphic variants, variants with conservative substitutions, and the like) and domains from alternative species (e.g., mouse, rat). Typically, PDZ domains are substantially identical to those shown in U.S. Ser. No. 09/724,553, e.g., at least about 70%, at least about 80%, or at least about 90% amino acid residue identity when compared and aligned for maximum correspondence. The percentage of sequence identity, also termed homology, between a polypeptide native and a variant sequence may be determined by comparing the two sequences using the GAP program (Wisconsin Sequence Analysis Package, Version 8 for Unix, Genetics Computer Group, University Research Park, Madison Wis.), which uses the algorithm of Smith and Waterman (1981). It is appreciated in the art that PDZ domains can be mutated to give amino acid changes that can strengthen or weaken binding and to alter specificity, yet they remain PDZ domains (Schneider et al. 1998). Unless otherwise indicated, a reference to a particular PDZ domain (e.g., KIAAO751 or PRIL-16 d1) is intended to encompass the particular PDZ domain and variants that bind the same relevant protein ligand as the native protein, (e.g., MUC1-binding variants of KIAAO751 or PRIL-16 d1). In other words, if a reference is made to a particular PDZ domain, a reference is also made to variants of that PDZ domain wherein the variant is competent to bind the relevant protein ligand, e.g., cytoplasmic tail of MUC1, as described herein.

As used herein, the term “PDZ protein” refers to a naturally-occurring protein containing a PDZ domain. Exemplary PDZ proteins include ZO-1, SIP1, LIM MYSTIQUE, AIPC, KIAA0751, MAST2, PRIL-16, GRIP2, SITAC 18, NSP, and KIAA1526.

As used herein, the term “PDZ-domain polypeptide” refers to a polypeptide containing a PDZ domain, such as a fusion protein including a PDZ domain sequence, a naturally-occurring PDZ protein, or an isolated PDZ domain peptide. A PDZ-domain polypeptide may therefore be about 60 amino acids or more in length, about 70 amino acids or more in length, about 80 amino acids or more in length, about 90 amino acids or more in length, about 100 amino acids or more in length, about 200 amino acids or more in length, about 300 amino acids or more in length, about 500 amino acids or more in length, about 800 amino acids or more in length, about 1000 amino acids or more in length, usually up to about 2000 amino acids or more in length. PDZ domain peptides are usually no more than about 100 amino acids (e.g., 50-60 amino acids, 60-70 amino acids, 80-90 amino acids, or 90-100 amino acids), and encode a PDZ domain.

As used herein, the term “PL protein” or “PDZ Ligand protein” refers to a naturally-occurring protein that forms a molecular complex with a PDZ-domain, or to a protein whose carboxy-terminus, when expressed separately from the full length protein (e.g., as a peptide fragment of 4-25 residues, e.g., 8, 10, 12, 14 or 16 residues), forms such a molecular complex. The molecular complex can be observed in vitro using the binding assays described herein This definition is not intended to include anti-PDZ antibodies and the like.

As used herein, a “PL sequence” refers to the amino acid sequence of the C-terminus of a PL protein (e.g., the C-terminal 3, 4, 5, 6, 7, 8, 9, 10, 12, 14, 16, 20 or 25 residues) (“C-terminal PL sequence”) or to an internal sequence known to bind a PDZ domain (“internal PL sequence”).

As used herein, a “PL peptide” is a peptide of having a sequence from, or based on, the sequence of the C-terminus of a PL protein. Exemplary MUC1 PL peptides (biotinylated) are listed in Table 8.

As used herein, a “PL fusion protein” is a fusion protein that has a PL sequence as one domain, typically as the C-terminal domain of the fusion protein. An exemplary PL fusion protein is a TAT-PL sequence fusion.

As used herein, the term “PL inhibitor peptide sequence” refers to PL peptide amino acid sequence that (in the form of a peptide or PL fusion protein) inhibits the interaction between a PDZ domain polypeptide and a PL peptide (e.g., as measured by the binding assays described herein).

As used herein, a “PDZ-domain encoding sequence” means a segment of a polynucleotide encoding a PDZ domain. In various embodiments, the polynucleotide is DNA, RNA, single-stranded or double-stranded.

As used herein, the terms “antagonist” and “inhibitor,” when used in the context of modulating a binding interaction (such as the binding of a PDZ domain sequence to a PL sequence), are used interchangeably and refer to an agent that reduces the binding of the, e.g., PL sequence (e.g., PL peptide) and the, e.g., PDZ domain sequence (e.g., PDZ protein, PDZ domain peptide).

As used herein, the terms “peptide mimetic,” “peptidomimetic,” and “peptide analog” are used interchangeably and refer to a synthetic chemical compound that has substantially the same structural and/or functional characteristics of a PL inhibitory or PL binding peptide of the invention. The mimetic can be either entirely composed of synthetic, non-natural analogues of amino acids, or, is a chimeric molecule of partly natural peptide amino acids and partly non-natural analogs of amino acids. The mimetic can also incorporate any amount of natural amino acid conservative substitutions as long as such substitutions also do not substantially alter the mimetic's structure and/or inhibitory or binding activity. As with polypeptides of the invention which are conservative variants, routine experimentation will determine whether a mimetic is within the scope of the invention, i.e., that its structure and/or function is not substantially altered. Thus, a mimetic composition is within the scope of the invention if it is capable of binding to a PDZ domain and/or inhibiting a PL-PDZ interaction.

Polypeptide mimetic compositions can contain any combination of nonnatural structural components, which are typically from three structural groups: a) residue linkage groups other than the natural amide bond (“peptide bond”) linkages; b) non-natural residues in place of naturally occurring amino acid residues; or c) residues which induce secondary structural mimicry, i.e., to induce or stabilize a secondary structure, e.g., a beta turn, gamma turn, beta sheet, alpha helix conformation, and the like.

A polypeptide can be characterized as a mimetic when all or some of its residues are joined by chemical means other than natural peptide bonds. Individual peptidomimetic residues can be joined by peptide bonds, other chemical bonds or coupling means, such as, e.g., glutaraldehyde, N-hydroxysuccinimide esters, bifunctional maleimides, N,N=-dicyclohexylcarbodiimide (ICC) or N,N=diisopropylcarbodiimide (DIC). Linking groups that can be an alternative to the traditional amide bond (“peptide bond”) linkages include, e.g., ketomethylene (e.g., —C(═O)—CH₂— for —C(═O)—NH—), aminomethylene (CH₂—NH), ethylene, olefin (CH═CH), ether (CH₂—O), thioether (CH₂—S), tetrazole (CN₄—), thiazole, retroamide, thioamide, or ester (see, e.g., Spatola (1983) in Chemistry and Biochemistry of Amino Acids, Peptides and Proteins, Vol. 7, pp 267-357, A Peptide Backbone Modifications, Marcell Dekker, N.Y.).

A polypeptide can also be characterized as a mimetic by containing all or some non-natural residues in place of naturally-occurring amino acid residues. Nonnatural residues are well described in the scientific and patent literature; a few exemplary nonnatural compositions useful as mimetics of natural amino acid residues and guidelines are described below.

Mimetics of aromatic amino acids can be generated by replacing by, e.g., D- or L-naphylalanine; D- or L-phenylglycine; D- or L-2 thieneylalanine; D- or L-1, -2, 3-, or 4-pyreneylalanine; D- or L-3 thieneylalanine; D- or L-(2-pyridinyl)-alanine; D- or L-(3-pyridinyl)-alanine, D- or L-(2-pyrazinyl)-alanine; D- or L-(4-isopropyl)-phenylglycine; D-(trifluoromethyl)-phenylglycine; D-(trifluoromethyl)-phenylalanine; D-p-fluorophenylalanine; D- or L-p-biphenylphenylalanine; K- or L-p-methoxybiphenylphenylalanine; D- or L-2-indole(alkyl)alanines; and, D- or L-alkylainines, where alkyl can be substituted or unsubstituted methyl, ethyl, propyl, hexyl, butyl, pentyl, isopropyl, iso-butyl, sec-isotyl, iso-pentyl, or a non-acidic amino acids. Aromatic rings of a nonnatural amino acid include, e.g., thiazolyl, thiophenyl, pyrazolyl, benzimidazolyl, naphthyl, furanyl, pyrrolyl, and pyridyl aromatic rings.

Mimetics of acidic amino acids can be generated by substitution by, e.g., non-carboxylate amino acids while maintaining a negative charge; (phosphono)alanine; sulfated threonine. Carboxyl side groups (e.g., aspartyl or glutamyl) can also be selectively modified by reaction with carbodiimides (R═—N—C—N—R═) such as, e.g., 1-cyclohexyl-3(2-morpholinyl-(4-ethyl) carbodiimide or 1-ethyl-3(4-azonia-4,4-dimetholpentyl) carbodiimide. Aspartyl or glutamyl can also be converted to asparaginyl and glutaminyl residues by reaction with ammonium ions.

Mimetics of basic amino acids can be generated by substitution with, e.g., (in addition to lysine and arginine) the amino acids ornithine, citrulline, or (guanidino)-acetic acid, or (guanidino)alkyl-acetic acid, where alkyl is defined above. Nitrile derivative (e.g., containing the CN-moiety in place of COOH) can be substituted for asparagine or glutamine. Asparaginyl and glutaminyl residues can be deaminated to the corresponding aspartyl or glutamyl residues.

Arginine residue mimetics can be generated by reacting arginyl with, e.g., one or more conventional reagents, including, e.g., phenylglyoxal, 2,3-butanedione, 1,2-cyclohexanedione, or ninhydrin, preferably under alkaline conditions.

Tyrosine residue mimetics can be generated by reacting tyrosyl with, e.g., aromatic diazonium compounds or tetranitromethane. N-acetylmidizol and tetranitromethane can be used to form O-acetyl tyrosyl species and 3-nitro derivatives, respectively.

Cysteine residue mimetics can be generated by reacting cysteinyl residues with, e.g., alpha-haloacetates such as 2-chloroacetic acid or chloroacetamide and corresponding amines, to give carboxymethyl or carboxyamidomethyl derivatives. Cysteine residue mimetics can also be generated by reacting cysteinyl residues with, e.g., bromo-trifluoroacetone, alpha-bromo-beta-(5-imidozoyl) propionic acid; chloroacetyl phosphate, N-alkylmaleimides, 3-nitro-2-pyridyl disulfide; methyl 2-pyridyl disulfide; p-chloromercuribenzoate; 2-chloromercuri-4 nitrophenol; or, chloro-7-nitrobenzo-oxa-1,3-diazole.

Lysine mimetics can be generated (and amino terminal residues can be altered) by reacting lysinyl with, e.g., succinic or other carboxylic acid anhydrides. Lysine and other alpha-amino-containing residue mimetics can also be generated by reaction with imidoesters, such as methyl picolinimidate, pyridoxal phosphate, pyridoxal, chloroborohydride, tritrobenzenesulfonic acid, O-methylisourea, 2,4, pentanedione, and transamidase-catalyzed reactions with glyoxylate.

Mimetics of methionine can be generated by reaction with, e.g., methionine sulfoxide. Mimetics of proline include, e.g., pipecolic acid, thiazolidine carboxylic acid, 3- or 4-hydroxy proline, dehydroproline, 3- or 4-methylproline, or 3,3,-dimethylproline. Histidine residue mimetics can be generated by reacting histidyl with, e.g., diethylprocarbonate or para-bromophenacyl bromide.

Other mimetics include, e.g., those generated by hydroxylation of proline and lysine; phosphorylation of the hydroxyl groups of seryl or threonyl residues; methylation of the alpha-amino groups of lysine, arginine and histidine; acetylation of the N-terminal amine; methylation of main chain amide residues or substitution with N-methyl amino acids; or amidation of C-terminal carboxyl groups.

A component of a natural polypeptide (e.g., a PL polypeptide or PDZ polypeptide) can also be replaced by an amino acid (or peptidomimetic residue) of the opposite chirality. Thus, any amino acid naturally-occurring in the L-configuration (which can also be referred to as the R or S, depending upon the structure of the chemical entity) can be replaced with the amino acid of the same chemical structural type or a peptidomimetic, but of the opposite chirality, generally referred to as the D-amino acid, but which can additionally be referred to as the R— or S— form.

The mimetics of the invention can also include compositions that contain a structural mimetic residue, particularly a residue that induces or mimics secondary structures, such as a beta turn, beta sheet, alpha helix structures, gamma turns, and the like. For example, substitution of natural amino acid residues with D-amino acids; N-alpha-methyl amino acids; C-alpha-methyl amino acids; or dehydroamino acids within a peptide can induce or stabilize beta turns, gamma turns, beta sheets or alpha helix conformations. Beta turn mimetic structures have been described, e.g., by Nagai, (1985); Feigl (1986); Kahn (1988); Kemp (1988); Kahn (1988a). Beta sheet mimetic structures have been described, e.g., by Smith (1992). For example, a type VI beta turn induced by a cis amide surrogate, 1,5-disubstituted tetrazol, is described by Beusen (1995). Incorporation of achiral omega-amino acid residues to generate polymethylene units as a substitution for amide bonds is described by Banerjee (1996). Secondary structures of polypeptides can be analyzed by, e.g., high-field 1H NMR or 2D NMR spectroscopy, see, e.g., Higgins (1997). See also, Hruby (1997) and Balaji et al., U.S. Pat. No. 5,612,895.

As used herein, “peptide variants” and “conservative amino acid substitutions” refer to peptides that differ from a reference peptide (e.g., a peptide having the sequence of the carboxy-terminus of a specified PL protein) by substitution of an amino acid residue having similar properties (based on size, polarity, hydrophobicity, and the like). Thus, insofar as the compounds that are encompassed within the scope of the invention are partially defined in terms of amino acid residues of designated classes, the amino acids may be generally categorized into three main classes: hydrophilic amino acids, hydrophobic amino acids and cysteine-like amino acids, depending primarily on the characteristics of the amino acid side chain. These main classes may be further divided into subclasses. Hydrophilic amino acids include amino acids having acidic, basic or polar side chains and hydrophobic amino acids include amino acids having aromatic or apolar side chains. Apolar amino acids may be further subdivided to include, among others, aliphatic amino acids. The definitions of the classes of amino acids as used herein are as follows:

“Hydrophobic Amino Acid” refers to an amino acid having a side chain that is uncharged at physiological pH and that is repelled by aqueous solution. Examples of genetically encoded hydrophobic amino acids include Ile, Leu and Val. Examples of non-genetically encoded hydrophobic amino acids include t-BuA.

“Aromatic Amino Acid” refers to a hydrophobic amino acid having a side chain containing at least one ring having a conjugated π-electron system (aromatic group). The aromatic group may be further substituted with groups such as alkyl, alkenyl, alkynyl, hydroxyl, sulfanyl, nitro and amino groups, as well as others. Examples of genetically encoded aromatic amino acids include Phe, Tyr and Trp. Commonly encountered non-genetically encoded aromatic amino acids include phenylglycine, 2-naphthylalanine, β-2-thienylalanine, 1,2,3,4-tetrahydroisoquinoline-3-carboxylic acid, 4-chloro-phenylalanine, 2-fluorophenyl-alanine, 3-fluorophenylalanine and 4-fluorophenylalanine.

“Apolar Amino Acid” refers to a hydrophobic amino acid having a side chain that is generally uncharged at physiological pH and that is not polar. Examples of genetically encoded apolar amino acids include Gly, Pro and Met. Examples of non-encoded apolar amino acids include Cha.

“Aliphatic Amino Acid” refers to an apolar amino acid having a saturated or unsaturated straight chain, branched or cyclic hydrocarbon side chain. Examples of genetically encoded aliphatic amino acids include Ala, Leu, Val and Ile. Examples of non-encoded aliphatic amino acids include Nle.

“Hydrophilic Amino Acid” refers to an amino acid having a side chain that is attracted by aqueous solution. Examples of genetically encoded hydrophilic amino acids include Ser and Lys. Examples of non-encoded hydrophilic amino acids include Cit and hCys.

“Acidic Amino Acid” refers to a hydrophilic amino acid having a side chain pK value of less than 7. Acidic amino acids typically have negatively charged side chains at physiological pH due to loss of a hydrogen ion. Examples of genetically encoded acidic amino acids include Asp and Glu.

“Basic Amino Acid” refers to a hydrophilic amino acid having a side chain pK value of greater than 7. Basic amino acids typically have positively charged side chains at physiological pH due to association with hydronium ion. Examples of genetically encoded basic amino acids include Arg, Lys and His. Examples of non-genetically encoded basic amino acids include the non-cyclic amino acids ornithine, 2,3-diaminopropionic acid, 2,4-diaminobutyric acid and homoarginine.

“Polar Amino Acid” refers to a hydrophilic amino acid having a side chain that is uncharged at physiological pH, but which has a bond in which the pair of electrons shared in common by two atoms is held more closely by one of the atoms. Examples of genetically encoded polar amino acids include Asx and Glx. Examples of non-genetically encoded polar amino acids include citrulline, N-acetyl lysine and methionine sulfoxide.

“Cysteine-Like Amino Acid” refers to an amino acid having a side chain capable of forming a covalent linkage with a side chain of another amino acid residue, such as a disulfide linkage. Typically, cysteine-like amino acids generally have a side chain containing at least one thiol (SH) group. Examples of genetically encoded cysteine-like amino acids include Cys. Examples of non-genetically encoded cysteine-like amino acids include homocysteine and penicillamine.

As will be appreciated by those having skill in the art, the above classification are not absolute, and several amino acids exhibit more than one characteristic property, and can therefore be included in more than one category. For example, tyrosine has both an aromatic ring and a polar hydroxyl group. Thus, tyrosine has dual properties and can be included in both the aromatic and polar categories. Similarly, in addition to being able to form disulfide linkages, cysteine also has apolar character. Thus, while not strictly classified as a hydrophobic or apolar amino acid, in many instances cysteine can be used to confer hydrophobicity to a peptide.

Certain commonly encountered amino acids which are not genetically encoded of which the peptides and peptide analogues of the invention may be composed include, but are not limited to, β-alanine (b-Ala) and other omega-amino acids such as 3-aminopropionic acid (Dap), 2,3-diaminopropionic acid (Dpr), 4-aminobutyric acid and so forth; α-aminoisobutyric acid (Aib); ε-aminohexanoic acid (Aha); Δ-aminovaleric acid (Ava); N-methylglycine or sarcosine (MeGly); ornithine (Orn); citrulline (Cit); t-butylalanine (t-BuA); t-butylglycine (t-BuG); N-methylisoleucine (MeIle); phenylglycine (Phg); cyclohexylalanine (Cha); norleucine (Nle); 2-naphthylalanine (2-Nal); 4-chlorophenylalanine (Phe(4-Cl)); 2-fluorophenylalanine (Phe(2-F)); 3-fluorophenylalanine (Phe(3-F)); 4-fluorophenylalanine (Phe(4-F)); penicillamine (Pen); 1,2,3,4-tetrahydroisoquinoline-3-carboxylic acid (Tic); β-2-thienylalanine (Thi); methionine sulfoxide (MSO); homoarginine Arg); N-acetyl lysine (AcLys); 2,3-diaminobutyric acid (Dab); 2,3-diaminobutyric acid (Dbu); p-aminophenylalanine (Phe(pNH₂)); N-methyl valine (MeVal); homocysteine (hCys) and homoserine (hSer). These amino acids also fall conveniently into the categories defined above.

The classifications of the above-described genetically encoded and non-encoded amino acids are summarized in Table 1, below. It is to be understood that Table 1 is for illustrative purposes only and does not purport to be an exhaustive list of amino acid residues which may comprise the peptides and peptide analogues described herein. Other amino acid residues which are useful for making the peptides and peptide analogues described herein can be found, e.g., in Fasman, 1989, CRC Practical Handbook of Biochemistry and Molecular Biology, CRC Press, Inc., and the references cited therein. Amino acids not specifically mentioned herein can be conveniently classified into the above-described categories on the basis of known behavior and/or their characteristic chemical and/or physical properties as compared with amino acids specifically identified.

TABLE 1

| Classification | Genetically Encoded | Genetically Non-Encoded |
|---|---|---|
| Hydrophobic | | |
| Aromatic | F, Y, W | Phg, Nal, Thi, Tic, Phe(4-Cl), |
| | | Phe(2-F), Phe(3-F), Phe(4-F), |
| | | Pyridyl Ala, Benzothienyl Ala |
| Apolar | M, G, P | |
| Aliphatic | A, V, L, I | t-BuA, t-BuG, MeIle, Nle, MeVal, |
| | | Cha, bAla, MeGly, Aib |
| Hydrophilic | | |
| Acidic | D, E | |
| Basic | H, K, R | Dpr, Orn, hArg, Phe(p-NH₂), |
| | | DBU, A₂BU |
| Polar | Q, N, S, T, Y | Cit, AcLys, MSO, hSer |
| Cysteine-Like | C | Pen, hCys, p-methyl Cys |

Cyclic derivatives of the peptides of the invention are also part of the present invention. Cyclization may allow the peptide to assume a more favorable conformation for association with molecules in complexes of the invention. Cyclization may be achieved using techniques known in the art, e.g., disulfide bonds may be formed between two appropriately spaced components having free sulfhydryl groups, or an amide bond may be formed between an amino group of one component and a carboxyl group of another component. Cyclization may also be achieved using an azobenzene-containing amino acid as described by Ulysse et al. (1995). The side chains of tyrosine and asparagine may be linked to form cyclic peptides. The components that form the bonds may be side chains of amino acids, non-amino acid components or a combination of the two. In an embodiment of the invention, cyclic peptides are contemplated that have a beta-turn in the right position. Beta-turns may be introduced into the peptides of the invention by adding the amino acids proline and glycine at the right position.

In addition to novel peptides herein disclosed, some peptide sequences that bind to PDZ domains of interest have been previously disclosed, e.g., sequences SEQ ID NO: 173 through SEQ ID NO: 188 are disclosed in WO02311512, incorporated herein by reference, wherein the sequences bind to RIM2 and other PDZ domains, and SEQ ID NO: 189 and SEQ ID NO: 190 are disclosed in WO03014303, incorporated herein by reference, wherein the sequences bind to RIM2 and other PDZ domains binding sequences.

In some embodiments, the agent that inhibits MUC1 binding to a PDZ domain is a peptide of the formula X¹-aa²-aa¹-aa⁰ wherein aa² is a hydrophobic aliphatic amino acid residue or a hydrophobic aromatic amino acid residue, aa² is a hydrophobic aliphatic amino acid residue, hydrophobic aromatic amino acid residue, polar amino acid residue, basic amino acid residue or an acidic amino acid residue, aa¹ is an amino acid residue and X¹ is a sequence of 0 to 200 amino acid residues, or 0 to 100 amino acid residues, or 0 to 50 amino acid residues, or 0 to 25 amino acid residues. In some embodiments, X¹ is 1, 2, 3, 4, 5, 6, 7, 8, 9, 10, 11, 12, 13, 14, 15, 16, 17, 18, 19 or 20 amino acid residues. In some embodiments aa⁰ is V, L, A, I, S or Y and aa² is V, L, A, I, Y, W, Q, N, S, T, R, K, D or E. In some embodiments, the residues aa²-aa¹-aa⁰ of the peptide of the formula X¹-aa²-aa¹-aa⁰ is selected from SEQ ID NO: 1 through SEQ ID NO: 40: RIV (SEQ ID NO: 1); LYI (SEQ ID NO: 2); SVV (SEQ ID NO: 3); AEV (SEQ ID NO: 4); SQL (SEQ ID NO: 5); SAA (SEQ ID NO: 6); SDA (SEQ ID NO: 7); SLV (SEQ ID NO: 8); SGI (SEQ ID NO: 9); SKV (SEQ ID NO: 10); FYA (SEQ ID NO: 11); TRV (SEQ ID NO: 12); TTL (SEQ ID NO: 13); TDV (SEQ ID NO: 14); SDV (SEQ ID NO: 15); YFI (SEQ ID NO: 16); YYV (SEQ ID NO: 17); ELV (SEQ ID NO: 18); IWA (SEQ ID NO: 19); ANL (SEQ ID NO: 20); IIA (SEQ ID NO: 21); RIA (SEQ ID NO: 22); YWA (SEQ ID NO: 23); IWS (SEQ ID NO: 24); INL (SEQ ID NO: 25); IRV (SEQ ID NO: 26); VEV (SEQ ID NO: 27); YIV (SEQ ID NO: 28); YQI (SEQ ID NO: 29); LML (SEQ ID NO: 30); VPV (SEQ ID NO: 31); IVL (SEQ ID NO: 32); VSL (SEQ ID NO: 33); VWV (SEQ ID NO: 34); EYV (SEQ ID NO: 35); EIV (SEQ ID NO: 36); IIY (SEQ ID NO: 37); IUV (SEQ ID NO: 38); TWV (SEQ ID NO: 39); and TQV (SEQ ID NO: 40).

In some embodiments, the peptide of formula X¹-aa²-aa¹-aa⁰ comprises as the carboxy-terminus the carboxy-terminal 4, 5 6, 7, 8 or 9 residues of a nine amino acid residue sequence selected from SEQ ID NO: 41 through SEQ ID NO: 94: ARGDRKRIV (SEQ ID NO: 41); TLASHQLYI (SEQ ID NO: 42); GMTSSSSVV (SEQ ID NO: 43); YGSPRYAEV (SEQ ID NO: 44); WPPSSSSQL (SEQ ID NO: 45); DDYDDISAA (SEQ ID NO: 46); LKPPATSDA (SEQ ID NO: 47); DKERLTSDA (SEQ ID NO: 48); FRNETQSLV (SEQ ID NO: 49); ALRASESGI (SEQ ID NO: 50); LVEAQKSKV (SEQ ID NO: 51); PTKQEEFYA (SEQ ID NO: 52); FSRRPKTRV (SEQ ID NO: 53); SSGHTSTTL (SEQ ID NO: 54); NIKKIFTDV (SEQ ID NO: 55); KMDSIESDV (SEQ ID NO: 56); DSSRKEYFI (SEQ ID NO: 57); KNKDKEYYV (SEQ ID NO: 58); VTDHKTELV (SEQ ID NO: 59); QDEEEGIWA (SEQ ID NO: 60); AVAATSINL (SEQ ID NO: 61); AVAATYSNL (SEQ ID NO: 62); ARGDRKRWA SEQ ID NO: 63); ARGDRKRWL (SEQ ID NO: 64); AVAATGIWA (SEQ ID NO: 65); QDEEETIVA (SEQ ID NO: 66); ARSDRTIWA (SEQ ID NO: 67); ARSDRTIIA (SEQ ID NO: 68); ARSDRKRIA (SEQ ID NO: 69); SRTDRKYWA (SEQ ID NO: 70); QDEEEGIWS (SEQ ID NO: 71); SRTVREIWA (SEQ ID NO: 72); SVTSTSINL (SEQ ID NO: 73); ARGDRKIRV (SEQ ID NO: 74); ARTDRKVEV (SEQ ID NO: 75); ARGDRKYIV (SEQ ID NO: 76); SRTDRKYQI (SEQ ID NO: 77); ARGDVRLML (SEQ ID NO: 78); ARGDRKVPV (SEQ ID NO: 79); QDERRLIVL (SEQ ID NO: 80); ARGDRLVSL (SEQ ID NO: 81); ARGTRLVWV (SEQ ID NO: 82); ARGDRYRUV (SEQ ID NO: 83); SRTDRLEYV (SEQ ID NO: 84); ARGDRLEIV (SEQ ID NO: 85); ARGDRTIIY (SEQ ID NO: 86); ARGDRRRUV (SEQ ID NO: 87); ARGDRKKUV (SEQ ID NO: 88); ARSDRKRIV (SEQ ID NO: 89); KNKDKEYYV (SEQ ID NO: 90); GMTSSSSVV (SEQ ID NO: 91); ARGRRETWV (SEQ ID NO: 92); QDERVETRV (SEQ ID NO: 93); and LQRRRETQV (SEQ ID NO: 94).

In some embodiments, the peptide of formula X¹-aa²-aa¹-aa⁰ comprises as the carboxy-terminus the carboxy-terminal 4, 5 6, 7, 8, 9, 10, 11, 12, 13, 14, 15, 16, 17, 18, 19 or 20 amino acid residues of NGGSSLSYTNPAVAAASANL (SEQ ID NO: 95) or NGGSSLSYTNPAVAATSANL (SEQ ID NO: 96).

In some embodiments, the amino-terminus of X¹ comprises X²-X³, wherein X² is a transmembrane transporter peptide sequence and X³ is an optional linker sequence. In some embodiments, the transmembrane transporter peptide sequence is derived from the Drosophila antennapedia protein, or homologs thereof. The 60 amino acid long homeodomain of the homeo-protein antennapedia has been demonstrated to translocate through biological membranes and can facilitate the translocation of coupled peptides. See for example Derossi et al. (1994) and Perez et al. (1992). It has been demonstrated that fragments as small as 16 amino acids long of this protein are sufficient to drive internalization. Examples of transmembrane transporter peptide sequences derived in unmodified or modified form from antennapedia include: RQIKIWFQNRRMKWKK (SEQ ID NO: 97) (Derossi et al., 1994); SGRQIKIWFQNRRMKWKKC (SEQ ID NO: 98) (Console et al., 2003); RRWRRWWRRWWRRWRR (SEQ ID NO: 99) (Williams et al., 1997); KKWKMRRNQFWIKIQR (SEQ ID NO: 100) (Derossi et al., 1996); and KKWKMRRNQFWIKIQR (SEQ ID NO: 101) (Pescarolo et al., 2001). The present invention contemplates a PDZ inhibitory peptide or peptidomirnmetic sequence as described herein, and at least a portion of the Antennapedia protein (or homolog thereof) sufficient to increase the transmembrane transport of the chimeric protein, relative to the PDZ inhibitory peptide or peptidomimetic, by a statistically significant amount.

Another example of an internalizing peptide is the HIV transactivator (TAT) protein. This protein appears to be divided into four domains Kuppuswamy et al., 1989). Purified TAT protein is taken up by cells in tissue culture (Frankel and Pabo, 1989), and peptides, such as the fragment corresponding to residues 37-62 of TAT, are rapidly taken up by cell in vitro (Green and Loewenstein, 1989). The highly basic region mediates internalization and targeting of the internalizing moiety to the nucleus (Ruben et al., 1989). Examples of transmembrane transporter peptide sequences derived in unmodified or modified form from TAT include YGRKKRRQRRR (SEQ ID NO: 102) (Vives et al., 1997); GRRKRRQRRRPPQ (SEQ ID NO: 103) (all L or all D amino acids) (Futaki et al., 2001); SGYGRKKRRQRRRC (SEQ ID NO: 104) (Console et al., 2003); RRRQRRKKRGY (SEQ ID NO: 105) (D amino acids) (Snyder et al., 2004); YARAAARQARA (SEQ ID NO: 106) (Ho et al., 2001); RKKRRQRRR (SEQ ID NO: 107) (Wender et al., 2000); RRRRRRRRR (SEQ ID NO: 108) (using either all L or all D amino acids) (Wender et al., 2000); RRRRRR (SEQ ID NO: 109) (Futaki et al., 2001); RRRRRRRR (SEQ ID NO: 110) (Futaki et al., 2001); and RRRQRR (SEQ ID NO: 111) (all D amino acids) (WO03059942). In some embodiments the peptide of formula X¹-aa²-aa¹-aa⁰ comprising a TAT transmembrane transporter peptide sequence selected from SEQ ID NO: 134 through 172.

Transmembrane transporter peptide sequences such as those derived from TAT and Antennapedia protein can also be attached to liposomes and the PDZ inhibitory peptide is translocated within the liposome (Torchilin & Levchenko, 2003; Tseng et al., 2002)

Other transmembrane transporter peptide sequences include galanin and mastoparan chimera sequences, e.g., GWTLNSAGYLLGKINLKALAALAKKIL (SEQ ID NO: 112) (Pooga et al., 1998) and AGYLLGKINLKALAALAKKIL (SEQ ID NO: 113) (Soomets et al., 2000); Herpes Simplex Virus VP22 derived sequences, e.g., DAATATRGRSAASRPTERPRAPARSASRPRRVE (SEQ ID NO: 114) (Elliot & O'Hare, 1997) and GALFLGFLGAAGSTMGAWSQPKSKRKV (SEQ ID NO: 115) (Morris et al., 1997); pegelin derived sequences, e.g., RGGRLSYSRRRFSTSTGR (SEQ ID NO:116) (Rousselle et al., 2000); integrin β3 signal derived sequences, e.g., VTVVLALGALAGVGVG (SEQ ID NO:117) (Liu et al., 1996); Karposi FGF signal derived sequences, e.g., AAVALLPAVLLALLAP (SEQ ID NO: 118) (Lin et al., 1996); amphipathic peptide sequences, e.g., KLALKLALKALKAALKLA (SEQ ID NO: 119) (Oehlke et al., 1998), FHV coat derived sequences, e.g., RRRRNRTRRNRRRVR (SEQ ID NO: 120) (Suzuki et al., 2002); synthetic sequences, e.g., PIRRRKLRRLK (SEQ ID NO: 121) and RRQRRTSKLMKR (SEQ ID NO: 122) (Mi et al., 2000); VE cadherin derived sequences. e.g., LLILRRRIRKQAHAHSK (SEQ ID NO: 123) (Elmquist et al., 2001) and nuclear localization signal derived sequences, e.g., SV40-NLS PKKKRKV (SEQ ID NO: 124) and Nucleoplasmin-NLS KRPAAIKKAGQAKKKK (SEQ ID NO: 125) (Futaki et al., 2001).

While not wishing to be bound by any particular theory, it is noted that hydrophilic polypeptides may be also be physiologically transported across the membrane barriers by coupling or conjugating the polypeptide to a transportable peptide which is capable of crossing the membrane by receptor-mediated transcytosis. Suitable internalizing peptides of this type can be generated using all or a portion of, e.g., a histone, insulin, transferrin, basic albumin, prolactin and insulin-like growth factor I (IGF-I), insulin-like growth factor II (IGF-II) or other growth factors. For instance, it has been found that an insulin fragment, showing affinity for the insulin receptor on capillary cells, and being less effective than insulin in blood sugar reduction, is capable of transmembrane transport by receptor-mediated transcytosis and can therefore serve as an internalizing peptide for the subject transcellular peptides and peptidomimetics. Preferred growth factor-derived internalizing peptides include EGF (epidermal growth factor)-derived peptides, such as CMHIESLDSYTC (SEQ ID NO: 126) and CMYIEALDKYAC (SEQ ID NO: 127); TGF-β (transforming growth factor beta)-derived peptides; peptides derived from PDGF (platelet-derived growth factor) or PDGF-2; peptides derived from IGF-I (insulin-like growth factor) or IGF-II; and FGF (fibroblast growth factor)-derived peptides. Also included are antibodies to receptors that are internalized upon binding of the antibody. Such antibodies include, but are not limited to, those that target MUC1, MUC4, EGRF, ErbB2, c-Met, GM-CSF alpha and beta receptors, bFGF receptors, TNF receptors, TGF beta receptor I-III, estrogen receptors, and G-protein coupled receptors.

Another class of translocating/internalizing peptides exhibits pH-dependent membrane binding. For an internalizing peptide that assumes a helical conformation at an acidic pH, the internalizing peptide acquires the property of amphiphilicity, e.g., it has both hydrophobic and hydrophilic interfaces. More specifically, within a pH range of approximately 5.0-5.5, an internalizing peptide forms an alpha-helical, amphiphilic structure that facilitates insertion of the moiety into a target membrane. An alpha-helix-inducing acidic pH environment may be found, for example, in the low pH environment present within cellular endosomes. Such internalizing peptides can be used to facilitate transport of PDZ inhibitory peptides and peptidomimetics, taken up by an endocytic mechanism, from endosomal compartments to the cytoplasm.

A preferred pH-dependent membrane-binding internalizing peptide includes a high percentage of helix-forming residues, such as glutamate, methionine, alanine and leucine. In addition, a preferred internalizing peptide sequence includes ionizable residues having pKa's within the range of pH 5-7, so that a sufficient uncharged membrane-binding domain will be present within the peptide at pH 5 to allow insertion into the target cell membrane.

Another preferred pH-dependent membrane-binding internalizing peptide in this regard is aa1-aa2-aa3-EAALA(EALA)4-EALEALAA-amide, which represents a modification of the peptide sequence of Subbarao et al. (1987). Within this peptide sequence, the first amino acid residue (aa1) is preferably a unique residue, such as C or K, that facilitates chemical conjugation of the internalizing peptide to a targeting protein conjugate. Amino acid residues 2-3 may be selected to modulate the affinity of the internalizing peptide for different membranes. For instance, if both residues 2 and 3 are K or R, the internalizing peptide will have the capacity to bind to membranes or patches of lipids having a negative surface charge. If residues 2-3 are neutral amino acids, the internalizing peptide will insert into neutral membranes.

Yet other preferred internalizing peptides include peptides of apo-lipoprotein A-1 and B; peptide toxins, such as melittin, bombolittin, delta hemolysin and the pardaxins; antibiotic peptides, such as alamethicin; peptide hormones, such as calcitonin, corticotrophin releasing factor, beta endorphin, glucagon, parathyroid hormone, pancreatic polypeptide; and peptides corresponding to signal sequences of numerous secreted proteins. In addition, exemplary internalizing peptides may be modified through attachment of substituents that enhance the alpha-helical character of the internalizing peptide at acidic pH.

Yet another class of internalizing peptides suitable for use within the present invention includes hydrophobic domains that are “hidden” at physiological pH, but are exposed in the low pH environment of the target cell endosome. Upon pH-induced unfolding and exposure of the hydrophobic domain, the moiety binds to lipid bilayers and effects translocation of the covalently linked polypeptide into the cell cytoplasm. Such internalizing peptides may be modeled after sequences identified in, e.g., Pseudomonas exotoxin A, clathrin, or Diphtheria toxin.

Pore-forming proteins or peptides may also serve as internalizing peptides herein. Pore-forming proteins or peptides may be obtained or derived from, for example, C9 complement protein, cytolytic T-cell molecules or NK-cell molecules. These moieties are capable of forming ring-like structures in membranes, thereby allowing transport of attached polypeptide through the membrane and into the cell interior.

Mere membrane intercalation of an internalizing peptide may be sufficient for translocation of the PDZ inhibitory peptide or peptidomimetic, across cell membranes. However, translocation may be improved by attaching to the internalizing peptide a substrate for intracellular enzymes (i.e., an “accessory peptide”). It is preferred that an accessory peptide be attached to a portion(s) of the internalizing peptide that protrudes through the cell membrane to the cytoplasmic face. The accessory peptide may be advantageously attached to one terminus of a translocating/internalizing moiety or anchoring peptide. An accessory moiety of the present invention may contain one or more amino acid residues. In one embodiment, an accessory moiety may provide a substrate for cellular phosphorylation (for instance, the accessory peptide may contain a tyrosine residue).

In embodiments wherein the amino terminus of X¹ comprises X²-X³, X³, the optional linker X³ may be any suitable flexible polylinker, including GGGGS (SEQ ID NO: 128) repeated 1 to 3 times (Huston et al., 1988); EGKSSGSGSESKVD (SEQ ID NO: 129) (Chaudhary et al., 1990); KESGSVSSEQLAQFRSLD (SEQ ID NO: 130) (Bird et al., 1988).

In some embodiments, the peptide of the formula X¹-aa²-aa¹-aa⁰ is a peptide of the formula X¹-aa⁸-aa⁷-aa⁶-aa⁵-aa⁴-aa³-aa²-aa¹-aa⁰, wherein X¹ is as defined previously and wherein in some embodiments is a peptide of the formula X²-aa⁸-aa⁷-aa⁶-aa⁵-aa⁴-aa³-aa²-aa¹-aa⁰, wherein, as defined previously, X² is a transmembrane transporter sequence, which in some embodiments is selected from SEQ ID NO: 95 through SEQ ID NO: 127, which in some embodiments is SEQ ID NO: 98, SEQ ID NO: 104 or SEQ ID NO: 119. In some embodiments aa¹ is a hydrophobic aromatic amino acid residue, which may be W or Y. In some embodiments aa⁴ is a basic amino acid residue or acidic amino acid residue, wherein in some embodiments, the basic amino acid residue is R and in some embodiments the acidic amino acid residue is E. In some embodiments, aa⁷ is an acidic, basic or hydrophobic aliphatic amino acid residue, wherein in some embodiments the basic amino acid residue is R, the acidic amino acid residue is D, and the hydrophobic aliphatic amino acid residue is V. In some embodiments, the peptide of formula X⁴-aa⁸-aa⁷-aa⁶-aa⁵-aa⁴-aa³-aa²-aa¹-aa⁰ is SEQ ID NO: 137, SEQ ID NO: 142, SEQ ID NO: 143, SEQ ID NO: 144, SEQ ID NO: 148, SEQ ID NO: 150, SEQ ID NO: 160, SEQ ID NO: 168, or SEQ ID NO: 170.

One aspect of the present invention encompasses compositions and pharmaceutical compositions of the forgoing described peptides that inhibit the binding of the cytoplasmic domain of MUC1 to one or more PDZ domains.

The polypeptides of the present invention can be created by synthetic techniques or recombinant techniques which employ genomic or cDNA cloning methods. Polypeptides can be routinely synthesized using solid phase or solution phase peptide synthesis. Methods of preparing relatively short polypeptides peptides, by chemical synthesis are well known in the art. Such polypeptides could, for example be produced by solid-phase peptide synthesis techniques using commercially available equipment and reagents such as those available from Milligen (Bedford, Mass.) or Applied Biosystems-Perkin Elmer Foster City, Calif.). Alternatively, segments of such polypeptides could be prepared by solid-phase synthesis and linked together using segment condensation methods such as those described by Dawson et al., (1994). During chemical synthesis of such polypeptides, substitution of any amino acid is achieved simply by replacement of the residue that is to be substituted with a different amino acid monomer.

### III. Combination with Chemotherapeutic Agents

The present invention encompasses the use of modulators of MUC1 mediated signal transduction of the present invention in combination with chemotherapeutic agents. While not being limited by any particular theory, MUC1 inhibits the apoptotic response to genotoxic stress induced by certain chemotherapeutic agents, and thereby induces resistance to such agents. Modulators of MUC1 mediated signal transduction may be used to mitigate this MUC1 mediated response to chemotherapeutic agents, thereby enhancing the effectiveness of such agents. In this regard, the compositions of the present invention will be useful for the treatment cancer cells resistant to chemotherapeutic agents, including residual cancers remaining or reoccurring after cancer chemotherapy. The foregoing rational also pertains to the combination of compositions of the present invention and ionizing radiation.

The chemotherapeutic agents useful in the methods of the invention include the full spectrum of compositions and compounds which are known to be active in killing and/or inhibiting the growth of cancer cells. The chemotherapeutic agents, grouped by mechanism of action include DNA-interactive agents, antimetabolites, tubulin interactive agents, anti-hormonals, anti-virals, ODC inhibitors and other cytotoxics such as hydroxy urea Any of these agents are suitable for use in the methods of the present invention. DNA-interactive agents include the alkylating agents, e.g., cisplatin, cyclophosphamide, altretamine; the DNA strand-breakage agents, such as bleomycin; the intercalating topoisomerase II inhibitors, e.g., dactinomycin and doxorubicin); the nonintercalating topoisomerase II inhibitors such as, etoposide and teniposide; and the DNA minor groove binder plicamycin.

The alkylating agents form covalent chemical adducts with cellular DNA, RNA and protein molecules and with smaller amino acids, glutathione and similar chemicals. Generally, these alkylating agents react with a nucleophilic atom in a cellular constituent, such as an amino, carboxyl, phosphate, sulfhydryl group in nucleic acids, proteins, amino acids, or glutathione. The mechanism and the role of these alkylating agents in cancer therapy is not well understood. Typical alkylating agents include: nitrogen mustards, such as chlorambucil, cyclophosphamide, ifosfamide, mechlorethamine, melphalan, uracil mustard; aziridine such as thiotepa; methanesulphonate esters such as busulfan; nitroso ureas, such as carmustine, lomustine, streptozocin; platinum complexes such as cisplatin, carboplatin; bioreductive alkylators, such as mitomycin and procarbazine, dacarbazine and altretemine; DNA strand-breaking agents including bleomycin.

Topoisomerases are ubiquitous cellular enzymes which initiate transient DNA strand breaks during replication to allow for free rotation of the strands. The functionality of these enzymes is critical to the replication process of DNA. Without them, the torsional strain in the DNA helix prohibits free rotation, the DNA strands are unable to separate properly, and the cell eventually dies without dividing. Topo I links to the 3′-terminus of a DNA single strand break, while Topo II links to the 5′-terminus of a double strand DNA break. DNA topoisomerase II inhibitors include intercalators such as amsacrine, dactinomycin, daunorubicin, doxorubicin, idarubicin and mitoxantrone; nonintercalators such as etoposide and teniposide; camptothecins including irinotecan (CPT-II) and topotecan. A representative DNA minor groove binder is plicamycin.

The antimetabolites generally exert cytotoxic activity by interfering with the production of nucleic acids by one or the other of two major mechanisms. Some of the drugs inhibit production of the deoxyribonucleoside triphosphates that are the immediate precursors of DNA synthesis, thus inhibiting DNA replication. Some of the compounds are sufficiently like purines or pyrimidines to be able to substitute for them in the anabolic nucleotide pathways. These analogs can then be substituted into the DNA and RNA instead of their normal counterparts. The antimetabolites useful herein include: folate antagonists such as methotrexate and trimetrexate; pyrimidine antagonists such as fluorouracil, fluorodeoxyuridine, azacitidine, cytarabine, and floxuridine; purine antagonists include mercaptopurine, 6-thioguanine, fludarabine, pentostatin; sugar modified analogs include cytarabine, fludarabine; ribonucleotide reductase inhibitors include hydroxyurea.

Tubulin interactive agents interfere with cell division by binding to specific sites on Tubulin, a protein that polymerizes to form cellular microtubules. Microtubules are critical cell structure units. When the interactive agents bind on the protein, the cell cannot properly form microtubules. Tubulin interactive agents include vincristine and vinblastine, both alkaloids and the taxanes (paclitaxel and docetaxel). Although their mechanisms of action are different, both taxanes and vinca alkaloids exert their biological effects on the cell microtubles. Taxanes act to promote the polymerization of tubulin, a protein subunit of spindle microtubles. The end result is the inhibition of depolymerization of the microtubles, which causes the formation of stable and nonfunctional microtubles. This disrupts the dynamic equilibrium within the microtuble system, and arrests the cell cycle in the late G₂ and M phases, which inhibits cell replication.

Like taxanes, vinca alkaloids also act to affect the microtuble system within the cells. In contrast to taxanes, vinca alkaloids bind to tubulin and inhibit or prevent the polymerization of tubulin subunits into microtubles. Vinca alkaloids also induce the depolymerization of microtubles, which inhibits microtuble assembly and mediates cellular metaphase arrest. Vinca alkaloids also exert effects on nucleic acid and protein synthesis; amino acid, cyclic AMP, and glutathione synthesis; cellular respiration; and exert immunosuppressive activity at higher concentrations.

Antihormonal agents exert cytotoxic activity by blocking hormone action at the end-receptor organ. Several different types of neoplasm require hormonal stimulation to propagate cell reproduction. The antihormonal agents, by blocking hormone action, deprive the neoplastic cells of a necessary stimulus to reproduce. As the cells reach the end of their life cycle, they die normally, without dividing and producing additional malignant cells. Antihormonal agents are typically derived from natural sources and include: estrogens, conjugated estrogens and ethinyl estradiol and diethylstibesterol, chlortrianisen and idenestrol; progestins such as hydroxyprogesterone caproate, medroxyprogesterone, and megestrol; androgens such as testosterone, testosterone propionate; fluoxymesterone, ethyltestosterone.

Adrenal corticosteroids are derived from natural adrenal cortisol or hydrocortisone. They are used because of their anti-inflammatory benefits as well as the ability of some to inhibit mitotic divisions and to halt DNA synthesis. These compounds include prednisone, dexamethasone, methylprednisolone, and prednisolone.

Leutinizing-releasing hormone agents or gonadotropin-releasing hormone antagonists are used primarily in the treatment of prostate cancer. These include leuprolide acetate and goserelin acetate. They prevent the biosynthesis of steroids in the testes.

Anti-hormonal agents include antiestrogenic agents such as tamoxifen, antiandrogen agents such as flutamide, and antiadrenal agents such as mitotane and aminoglutethimide.

ODC (or ornithine decarboxylase) inhibitors inhibit cancerous and pre-cancerous cell proliferation by depleting or otherwise interfering with the activity of ODC, the rate limiting enzyme of polyamine biosynthesis important to neoplastic cell growth. In particular, polyamine biosynthesis wherein ornithine is converted to the polyamine, putrescine, with putrescine being subsequently by converted to spermidine and spermine appears to be an essential biochemical event in the proliferation of neoplastic growth in a variety of cancers and cancer cell lines and the inhibition of ODC activity or depletion of ODC in such neoplastic cells has been shown to reduce polyamine levels in such cells leading to cell growth arrest; more differentiated cell morphology and even cellular senescence and death. In this regard, ODC or polyamine synthesis inhibitors are considered to be more cytotoxic agents functioning to prevent cancer reoccurrence or the conversion of pre-cancerous cells to cancerous cells than cytotoxic or cell killing agents. A suitable ODC inhibitor is eflornithine or α-difluoromethyl-ornithine, an orally available, irreversible ODC inhibitor, as well as a variety of polyamine analogs which are in various stages of pre-clinical and clinical research.

Other cytotoxics include agents which interfere or block various cellular processes essential for maintenance of cellular functions or cell mitosis as well as agents which promote apoptosis. In this regard, hydroxyurea appears to act via inhibitors of the enzyme ribonucleotide reductase whereas asparaginase enzymatically converts asparagine into non-functional aspartic acid thereby blocking protein synthesis in a tumor.

Compositions of the present invention can also be used in combination with antibodies to HER-2, such as Trastuzumab (Herceptin (I)). In addition, the present invention also encompasses the use of MUC1 domain antagonists in combination with epidermal growth factor receptor-interactive agents such as tyrosine kinase inhibitors. Tyrosine kinase inhibitors suitably include imatinib (Norvartis), OSI-774 (OSI Pharmaceuticals), ZD-1839 (AstraZeneca), SU-101 (Sugen) and CP-701 (Cephalon).

When used in the treatment methods of the present invention, it is contemplated that the chemotherapeutic agent of choice can be conveniently used in any formulation which is currently commercially available, and at dosages which fall below or within the approved label usage for single agent use.

### IV. Combination with Ionizing Radiation

In the present invention, the term “ionizing radiation” means radiation comprising particles or photons that have sufficient energy or can produce sufficient energy via nuclear interactions to produce ionization (gain or loss of electrons). An exemplary and preferred ionizing radiation is an x-radiation. Means for delivering x-radiation to a target tissue or cell are well known in the art. The amount of ionizing radiation needed in a given cell generally depends on the nature of that cell. Means for determining an effective amount of radiation are well known in the art. Used herein, the term “an effective dose” of ionizing radiation means a dose of ionizing radiation that produces cell damage or death when given in conjunction with the modulators of MUC1 mediated signal transduction of the present invention, optionally further combined with a chemotherapeutic agent.

Dosage ranges for x-rays range from daily doses of 50 to 200 roentgens for prolonged periods of time (3 to 4 weeks), to single doses of 2000 to 6000 roentgens. Dosage ranges for radioisotopes vary widely, and depend on the half-life of the isotope, the strength and type of radiation emitted, and the uptake by the neoplastic cells.

Any suitable means for delivering radiation to a tissue may be employed in the present invention, in addition to external means. For example, radiation may be delivered by first providing a radiolabeled antibody that immunoreacts with an antigen of the tumor, followed by delivering an effective amount of the radiolabeled antibody to the tumor. In addition, radioisotopes may be used to deliver ionizing radiation to a tissue or cell.

### V. Formulations

The compositions of the present invention such as peptides can be formulated in a variety of conventional pharmaceutical formulations and administered to cancer patients, in need of treatment, by any one of the drug administration routes conventionally employed including oral, intravenous, intraarterial, parental or intrapenitoneal.

For oral administration the compositions of the present invention may be formulated, for example, with an inert diluent or with an assimiable edible carrier, or enclosed in hard or soft shell gelatin capsules, or compressed into tablets, or incorporated directly with the food of the diet. For oral therapeutic administration, the active compound may be incorporated with excipients and used in the form of ingestible tablets, buccal tablets, troches, capsules, elixirs, suspensions, syrups, wafers, and the like. Such compositions and preparations may, of course, be varied and may conveniently be between about 2 to about 60% of the weight of the unit. The amount of active compounds in such therapeutically useful compositions is such that a suitable dosage will be obtained.

The tablets, troches, pills, capsules and the like may also contain the following: a binder, a gum tragacanth, acacia, cornstarch, or gelatin; excipients, such as dicalcium phosphate; a disintegrating agent, such as corn starch, potato starch, alginic acid and the like; a lubricant, such as magnesium stearate; and a sweetening agent, such as sucrose, lactose or saccharin may be added or a flavoring agent, such as peppermint, oil of wintergreen, or cherry flavoring. When the dosage unit for is a capsule, it may contain, in addition to materials of the above type, a liquid carrier. Various other materials may be present as coatings or to otherwise modify the physical form of the dosage unit. For instance, tablets, pills, or capsules may be coated with shellac, sugar or both. A syrup or elixir may contain the active compounds sucrose as a sweetening agent methyl and propylparabens as preservatives, a dye and flavoring, such as cherry or orange flavor. Of course, any material used in preparing a dosage unit form should be pharmaceutically pure and substantially non-toxic in the amounts employed. In addition, other chemotherapeutic compounds may be incorporated into sustained-release preparation and formulations.

Pharmaceutical formulations of the compositions of the present invention that are suitable for injectable use include sterile aqueous solutions or dispersions and sterile powders for the extemporaneous preparation of sterile injectable solutions or dispersions. In all cases the form must be sterile and must be fluid to the extent that syringability exists. It must be stable under the conditions of manufacture and storage and must be preserved against the contaminating action of microorganisms, such as bacteria and fungi. The carrier can be a solvent or dispersion medium containing, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions are prepared by incorporating the compositions of the present invention in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

As used herein, “pharmaceutically acceptable carrier” includes any and all solvents, dispersion media, coatings, antibacterial and antifungal agents, isotonic and absorption delaying agents and the like. The use of such media and agents for pharmaceutical active substances is well known in the art. Except insofar as any conventional media or agent is incompatible with the active ingredient, its use in the therapeutic compositions is contemplated. Supplementary active ingredients can also be incorporated into the composition.

### VI. Treatment Methods

Tumors that can be suitably treated with the methods of the present invention include; but are not limited to, tumors of the brain (glioblastomas, medulloblastoma, astrocytoma, oligodendroglioma, ependymomas), lung, liver, spleen, kidney, lymph node, small intestine, pancreas, blood cells, colon, stomach, breast, endometrium, prostate, testicle, ovary, skin, head and neck, esophagus, bone marrow, blood and other tissue. The tumor may be distinguished as metastatic and non-metastatic. Pre-malignant lesions may also be suitably treated with the methods of the present invention.

The treatment with modulators of compositions of the present invention may precede or follow irradiation and/or chemotherapy by intervals ranging from seconds to weeks and/or be administered concurrently with such treatments. In embodiments where the compositions of the present invention and irradiation and/or chemotherapy are applied separately to the cell, steps should be taken to ensure that a significant period of time does not expire between the time of each delivery, such that the combination of the two or three treatments would still be able to exert an advantageously combined effect on the cell. In such instances, it is contemplated that one would contact the cell with the treatment agents or modalities within amount 0.1 to 25 h of each other and, more preferably, within about 1 to 4 h of each other, with a delay time of only about 1 h to about 2 h being most preferred. In some situations, it is desirable to extend the time period of treatment significantly, however, where several days (2, 3, 4, 5, 6 or 7) or several weeks (1, 2, 3, 4, 5, 6, 7 or 8) lapse between the respective administrations. In any case, the invention contemplates that the compositions of the present invention may be given before, after or even simultaneously with the ionizing radiation and/or chemotherapeutic agent.

In the methods of the present invention, the actual dosage of compositions of the present invention employed will depend on a variety of factors including the type and severity of cancer being treated, and the additive or synergistic treatment effects of the compositions of the present invention and the other treatment modality or modalities selected.

### VII. Screening Methods

One aspect of the present invention is the use of screening methodologies, including high-throughput screens, related to the identification of compounds that modulate the binding of MUC1-CD to PDZ domains. Some embodiments utilize Omi/HtrA2, a MUC1-CD PDZ domain containing protein with serine protease activity that inhibits CIAP1, which is one of at least five human inhibitors of apoptosis (IAP) (Deveraux & Reed, 1999). The inhibition of CIAP1 is caused by cleavage at one of at least two sites, i.e., between amino acid residues 90 and 91 or 130 and 131 (numbering as per GenBank ND_{—}001157[gi:4502417] (Jin et al., 2003). The immediate amino acid residues adjacent to the cleavage points (denoted by ^{∇}) are: GLML^{∇}DNWK with L and D corresponding to amino acid residues 90 and 91, and NTSP^{∇}MRNS with P and M corresponding to amino acid residues 130 and 131. These and related peptides, such as 7-mers, 6-mers, 5-mers, 4-mers, and the like, may be used as model substrates in assays quantifying HtrA2 serine protease activity.

In some embodiments of the present invention, the aforementioned CIAP1 derived sequences are utilized in a homogeneous time-released fluorescence quenching assay (TR-FQA). The principal of such an assay is the use of a peptide substrate with a fluorescent tag, usually a europium chelate (e.g., LANCE, PerkinElmer Life and Analytical Sciences, Boston Mass.) coupled to one end and a quencher of the fluorescence, e.g., dabcyl, coupled to the other end. Upon cleavage of the peptide, the quencher will be separated and a time-resolved fluorescent signal is generated and quantified (see e.g., Karvinen et al., 2002, herein incorporated by reference). The peptides can be synthesized by standard FMOC chemistry (e.g., Applied Biosystems 433A peptide synthesizer, Foster City, Calif.). The building block for dabcyl is available from Molecular Probes (Eugene, Oreg.), and is used to prepare an intermediate peptide e.g., aminohexyl-LMLDNW-dabcyl-aminohexyl. The peptide intermediate is then labeled with the fluorescent europium chelate W1024 (PerkinElmer Life and Analytical Sciences, Boston Mass.). Peptide substrates are purified by conventional methods such as HPLC. Thus, one aspect of the present invention are substrate peptides: X¹-X²-LD-X³-X¹, wherein X¹ is a fluorescent label, which may be a europium chelate, which may be a europium isothiocyanate chelate, which may be W1024, X² is GLM, LM or M, X³ is DNW, DN or D and X⁴ is a dabcyl quenching group, or X¹-X⁵-PM-X⁶-X⁴, wherein X¹ and X⁴ are as described previously and X⁵ is NTS, TS or S and X⁶ is MRN, MR or M.

The foregoing substrates may be used to measure the activity of HtrA2, which may be a purified recombinant HtrA2. The full length human HtrA2 clone is available from the IMAGE consortium (GenBak AI979237-[gi:5804267]). GST fusion proteins may be used, the preparation and purification of such having been disclosed by Faccio et al. (2000), herein incorporated by reference. GST-HtrA2 fusion proteins may be attached to microbeads by methods known in the art. The assays may be undertaken in multi-well plates and time resolved florescence measured by suitable detector means such as a VICTOR V multilable counter or a ViewLux ultra-HTS microplate imager (PerkinElmer Life and Analytical Sciences, Boston Mass.). An alternative method using agarose sheets instead of multi-well plates has been described for Caspase-3 and may be adapted for HtrA2 (Sujatha et al., 2002, herein incorporated by reference).

HtrA2 promotes apoptosis (Martins, 2002) while MUC1 prevents apoptosis. Thus binding of MUC1 to the PDZ domain of HtrA2 should decrease the serine protease activity and consequently inhibit the ability of HtrA2 to inactivate CIAP1. Addition of MUC1-CD to the assay will therefore inhibit the time resolved florescent signal. This system can be used as a high-throughput-screen to select compounds for the ability to inhibit the MUC1-CD binding to the HtrA2 PDZ domain as indicated by the increase in the time resolved florescent signal.

### EXAMPLES OF THE INVENTION

### Example 1

### Requirement for Carboxy-Terminal Amino Acids for Co-Localization of MUC1 with FGFR3

293 cells were transiently transected with pIRESpuro2-Flag-MUC1-CD(1-72) or pIRESpuro2-Flag-MUC1-CD(1-68) that respectively expressed a full length MUC1 cytoplasmic domain: CQCRRKNYGQLDIFPARDTYHPMSEYPTYHTHGRYVPPSSTDRSPYEKVSAG NGGSSLSYTNPAVAATSANL (SEQ ID NO: 131) or a truncated domain formed by deletion of the four carboxy-terminal amino acids:

(SEQ ID NO: 132)

CQCRRKNYGQLDIFPARDTYHPMSEYPTYHTHGRYVPPSSTDRSPYEKVS
AGNGGSSLSYTNPAVAAT.

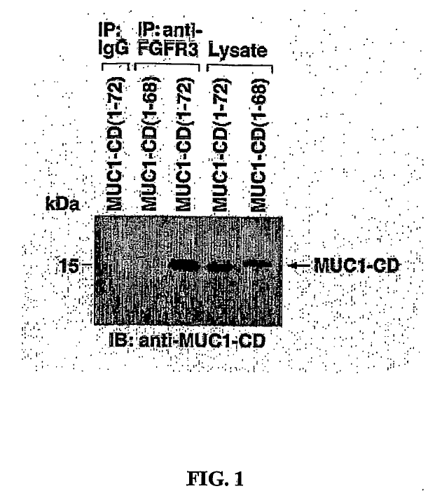

Cell lysates were prepared from subconfluent cells as described by Li et al. (1998). Equal amounts of cell lysate were incubated with anti-FGFR3 or mouse IgG. The immune complexes were prepared as described by Li et al. (1998), separated by SDS-PAGE and transferred to nitrocellulose membranes. The immunoblots were probed with anti-MUC1-CD (Neomarkers, Freemont Calif.). Lysates not subjected to immunoprecipitation were similarly analyzed by immunoblotting with anti-MUC1-CD. Reactivity was detected with a horseradish peroxidase-conjugated second antibody and chemiluminescence. The results are shown in FIG. 1 and indicate that deletion of the four MUC1 carboxy-terminal amino acid residues abolishes the ability of MUC1 to colocalize with FGFR3.

### Example 2

### Requirement for Carboxy-Terminal Amino Acids for Co-Localization of MUC1 with EGFR

293 cells were transiently transected with pIRESpuro2-Flag-MUC1-CD(1-72) or pIRESpuro2-Flag-MUC1-CD(1-68) that respectively expressed a MUC1 with a full length cytoplasmic domain: CQCRRKNYGQLDIFPARDTYHPMSEYPTYHTHGRYVPPSSTDRSPYEKVSAG NGGSSLSYTNPAVAATSANL (SEQ ID NO: 131) or a truncated domain formed by deletion of the four carboxy-terminal amino acids:

(SEQ ID NO: 132)

CQCRRKNYGQLDIFPARDTYHPMSEYPTYHTHGRYVPPSSTDRSPYEKVS
AGNGGSSLSYTNPAVAAT.

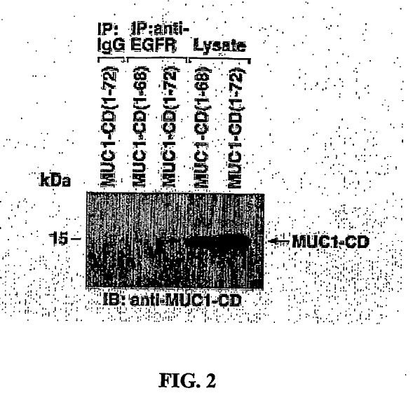

Cell lysates were prepared from subconfluent cells as described by Li et al. (1998). Equal amounts of cell lysate were incubated with anti-EGFR (Santa Cruz Biotechnology, Santa Cruz, Calif.) or mouse IgG. The immune complexes were prepared as described by Li et al. (1998), separated by SDS-PAGE and transferred to nitrocellulose membranes. The immunoblots were probed with anti-MUC1-CD (Neomarkers, Freemont Calif.). Lysates not subjected to immunoprecipitation were similarly analyzed by immunoblotting with anti-MUC1-CD. Reactivity was detected with a horseradish peroxidase-conjugated second antibody and chemiluminescence. The results are shown in FIG. 2 indicate that deletion of the four MUC1 carboxy-terminal amino acid residues abolishes the ability of MUC1 to colocalize with EGFR.

### Example 3

### Interaction of MUC1 with PDZ Domains

The ability of the MUC1 cytoplasmic domain (CD) to interact with a panel of 28 human PDZ domain proteins was screened. A His-tagged MUC1/CD was produced to affect the screening. The CD of MUC1 was amplified by RT-PCR from cDNA derived from human breast cancer MCF7 cells. The MUC1/CD gene was cloned into a bacterial vector pEXP (Panomics Inc.) to generate a His-tagged fusion protein (pEXP/MUC1/CD). DH5α E. Coli cells were transformed with pEXP/MUC1/CD and incubated overnight in 1 ml of LB medium containing 100 μg/ml ampicillin (LB/AMP). Eighty μl of the overnight culture was transferred to a tube of 4 ml of LB/AMP and allowed to grow until an OD₆₀₀ of approximately 0.5-0.8 was evident. IPTG was added to the culture at a final concentration of 0.5 mM to induce expression of the His-tagged MUC1/CD protein. After 4 hours, cells were harvested in 1× resuspension buffer (Panomics Inc.) and lysed by sonication. The lysate was centrifuged at 14000 rpm for 5 minutes at 4° C. The resulting supernatant (bacterial extract) was collected and stored at −80° C. until use.

The TransSignal PDZ Domain Array kit (Panomics Inc.) was used comprising membranes on which the following 28 human PDZ proteins had been immobilized: MINT-2 d1, Mint-3 d1, Mint-3 d2, Mint-1 d1, Mint1 d2, CSKP, Dlg d1, Dlg1 d3, Dlg2 d2, Dlg4 d3, DVL1, DVL3, DVLL, GIPC, HtrA2, LIMK2, MPP2, NEB1, OMP25, hCLIM1, PTPH1, ZO-2 d1, hPTP1E d1, hPTP1E d5, RGS12, RIL, ZO-1 d3 and ZO-2 d3.

Membranes were submerged in a small tray with 20 ml of x1 blocking buffer (Panomics Inc.), and shaken at room temperature for 1 hour. The blocking buffer was removed and membranes rinsed twice with x1 Wash Buffer (Panomics inc.) at room temperature. The bacterial extract was diluted to a final concentration of 0.1 mg/ml in 20 ml Resuspension Buffer (Panomics Inc.). The membrane was incubated with the dilute bacterial extract overnight at 4° C. with gentle shaking. After incubation, the membrane was washed three times with 40 ml 1× Wash Buffer at room temperature for 10 minutes each wash. The membrane was then incubated with 20 ml diluted Anti-Histidine HRP Conjugate (Panomics inc., 1:3000 dilution in 1× Wash Buffer) at room temperature for 1 hour. The membrane was then washed with 40 ml 1× Wash Buffer at room temperature for 10 minutes each wash. The membrane was then visualized using HYPERFILM ECL (Amersham Biosciences) utilizing the Detection Buffers as supplied by Panomics.

Binding was observed between his-tagged MUC1/CD and Mint-1 d2 (XII protein, PDZ domain #2), Mint-2 d1(XIIL protein, PDZ domain #1), HtrA2 (high temperature requirement protein A2), PTPH1 (protein-tyrosine phosphatase H1), RIL (reversion-induced LIM protein) and OMP25 (mitochondrial outer membrane protein 25).

### Example 4

### Inhibition of MUC1 Cytoplasmic Domain Binding to PDZ Domains

His-tagged MUC1/CD (bacterial extract) was incubated in the absence or presence of a 20-fold molar excess of the 7-mer peptide AAASANL (SEQ ID NO: 133) with the appropriate membrane-immobilized PDZ domain, as described above in Example 4. The results are summarized in Table 2.

TABLE 2
Inhibtion of Binding to MUC1/CD to Select PDZ Domains

Relative Binding of MUC1/CD

| | Protein conc. | no 7-mer | |
|---|---|---|---|
| PDZ | ng/spot | peptide | Plus 7-mer peptide |
| Mint-3 d1 | 400 | +++ | − |
| Mint-3 d1 | 80 | + | − |
| Mint-1 d2 | 400 | ++ | − |
| Mint-1 d2 | 80 | + | − |
| HTRA2 | 400 | ++ | − |
| HTRA2 | 80 | + | − |
| PTPH1 | 400 | + | − |
| PTPH1 | 80 | − | − |
| ZOP2 | 400 | − | − |
| ZOP2 | 80 | − | − |

The 7-mer, AAASANL (SEQ ID NO: 133), inhibited the binding of his-tagged MUC1/CD to PDZ domains Mint-1 d2, Mint-2 d1, HtrA2 PTPH1, RIL. The PDZ domain ZOP2 was used as a negative control.

### Example 5

### Deletion of MUC1 PDZ Ligand Domain Abrogates MUC1-Dependent Resistance to Cisplatin

Human HCT116 colon carcinoma cells (ATCC, Manassas, Va.) were cultured in Dulbecco's modified Eagle's medium/F12 with 10% heat-inactivated fetal bovine serum, 100 units/ml penicillin, 100 mg/ml streptomycin and 2 mM L-glutamine. HCT116 cells were transfected with pIRES-puro2, pIRESpuro2-MUC1 or pIRES-puro2-MUC1ΔSANL (MUC1 with the four carboxyl terminal amino acids SNAL deleted) as described (Li et al., 2001). Stable transfectants were selected in the presence of 0.4 mg/ml puromycin (Calbiochem-Novabiochem, San Diego, Calif.).

Cells were incubated with 100 μM cisplatin (CDDP; Sigma), for 24 and 48 hr. Visualization of viable cells indicated a substantially increase in the sensitivity to CDDP-induced cell death of HCT116 cells transfected with MUC1ΔSANL relative to cells transfected with full length MUC1. Data indicates that removal of the MUC1 carboxy-terminal PDZ ligand domain abrogates the ability of MUC1 to confer resistance to genotoxic agents.

### Example 6

### Preparation of Prokaryotic Expression Constructs Encoding PDZ Domains

PDZ domain containing genes or portions of PDZ domain containing genes were cloned into eukaryotic expression vectors in fusion with a glutathione S-transferase (GST) protein tags. Alternative tags include but are not limited to Enhanced Green Fluorescent Protein (EGFP), or Hemagglutinin (HA).

DNA fragments corresponding to PDZ domain containing genes were generated by RT-PCR from RNA from a library of individual cell line (CLONTECH Cat#K4000-1) derived RNA, using random (oligo-nucleotide) primers (Invitrogen Cat. #48190011). DNA fragments corresponding to PDZ domain-containing genes or portions of PDZ domain-containing genes were generated by standard PCR, using purified cDNA fragments (Table 3) and specific primers. Primers used were designed to create restriction nuclease recognition sites at the PCR fragment's ends, to allow cloning of those fragments into appropriate expression vectors. Subsequent to PCR, DNA samples were submitted to agarose gel electrophoresis. Bands corresponding to the expected size were excised. DNA was extracted by Sephaglas Band Prep Kit (Amersham Pharmacia Cat# 27-9285-01) and digested with appropriate restriction endonuclease. Digested DNA samples were purified once more by gel electrophoresis, according to the same protocol used above. Purified DNA fragments were coprecipitated and ligated with the appropriate linearized vector. After transformation into E. coli, bacterial colonies were screened by colony PCR and restriction digest for the presence and correct orientation of insert. Positive clones were innoculated in liquid culture for large scale DNA purification. The insert and flanking vector sites from the purified plasmid DNA were sequenced to ensure correct sequence of fragments and junctions between the vectors and fusion proteins.

TABLE 3
PDZ Domains Used in Screening Assays

| | | | |
|---|---|---|---|
| | GI or | | |
| Gene Name | Acc# | PDZ# | Sequence fused to GST Construct |

| | | | |
|---|---|---|---|
| 26s subunit | 9184389 | 1 | RDMAEAHKEAMSRKLGQSESQGPPRAFAKVNSISPGSPSIAGLQ |
| p27 | | | VDDEIVEFGSVNTQNFQSLHNIGSVVQHSEGALAPTILLSVSM |
| AF6 | 430993 | 1 | LRKEPEIITVTLKKQNGMGLSIVAAKGAGQDKLGIYVKSVVKGG |
| | | | AADVDGRLAAGDQLLSVDGRSLVGLSQERAAELMTRTSSVVTL |
| | | | EVAKQG |
| AIPC | 12751451 | 1 | LIRPSVISIIGLYKEKGKGLGFSIAGGRDCIRGQMGIFVKTIFPNGS |
| | | | AAEDGRLKEGDEILDVNGIPIKGLTFQEAIHTFKQIRSGLFVLTVR |
| | | | TKLVSPSLTNSS |
| AIPC | 12751451 | 2 | GISSLGRKTPGPKDRIVMEVTLNKEPRVGLGIGACCLALENSPPG |
| | | | IYIHSLAPGSVAKMESNLSRGDQILEVNSVNVRHAALSKVHAILS |
| | | | KCPPGPVRLVIGRHPNPKVSEQEMDEVIARSTYQESKEANSS |
| AIPC | 12751451 | 3 | QSENEEDVCFIVLNRKEGSGLGFSVAGGTDVEPKSITVHRVFSQ |
| | | | GAASQEGTMNRGDFLLSVNGASLAGLAHGNVLKVLHQAQLHK |
| | | | DALVVIKKGMDQPRPSNSS |
| AIPC | 12751451 | 4 | LGRSVAVHDALCVEVLKTSAGLGLSLDGGKSSVTGDGPLVIKR |
| | | | VYKGGAAEQAGIIEAGDEILAINGKPLVGLMHFDAWNIMKSVPE |
| | | | GPVQLLIRKHRNSS |
| alpha | 2773059 | 1 | REEGGMPQTVILPGPAAWGFRLSGGIDFNQPLVITRITPGSKAAA |
| actinin-2 | | | ANLCPGDVILAIDGFGTESMTHADGQDRIKAAAHQLCLKIDRGE |
| associated | | | THLWSPHSIV |
| LIM protein | | | |
| APXL-1 | 13651263 | 1 | ILVEVQLSGGAPWGFTLKGGREHGEPLVITKIEEGSKAAAVDKL |
| | | | LAGDEIVGINDIGLSGFRQEAICLVKGSHKTLKLVVKRNSS |
| Atrophin-1 | 2947231 | 1 | REKPLFTRDASQLKGTFLSTTLKKSNMGFGFTIIGGDEPDEFLQV |
| Interacting | | | KSVIPDGPAAQDGKMETGDVIVYINEVCVLGHTHADVVKLFQS |
| Protein | | | VPIGQSVNLVLCRGYP |
| Atrophin-1 | 2947231 | 2 | LSGATQAELMTLTIVKGAQGFGFTIADSPTGQRVKQILDIQGCPG |
| Interacting | | | LCEGDLIVEINQQNVQNLSHTEVVDILKDCPIGSETSLIIHRGGFF |
| Protein | | | |
| Atrophin-1 | 2947231 | 3 | HYKELDVHLRRMESGFGFRILGGDEPGQPILIGAVIAMGSADRD |
| Interacting | | | GRLHPGDELVYVDGIPVAGKTHRYVIDLMHHAARNGQVNLTV |
| Protein | | | RRKVLCG |
| Atrophin-1 | 2947231 | 4 | EGRGISSHSLQTSDAVIHRKENEGFGFVIISSLNRPESGSTITVPHK |
| Interacting | | | IGRIIDGSPADRCAKLKVGDRILAVNGQSIINMPHADIVKLIKDA |
| Protein | | | GLSVTLRIIPQEEL |
| Atrophin-1 | 2947231 | 5 | LSDYRQPQDFDYFTVDMEKGAKGFGFSIRGGREYKMDLYVLRL |
| Interacting | | | AEDGPAIRNGRMRVGDQIIEINGESTRDMTHARAIELIKSGGRRV |
| Protein | | | RLLLKRGTGQ |
| Atrophin-1 | 2947231 | 6 | HESVIGRNPEGQLGFELKGGAENGQFPYLGEVKPGKVAYESGS |
| Interacting | | | KLVSEELLLEVNETPVAGLTIRDVLAVIKHCKDPLRLKCVKQGG |
| Protein | | | IHR |
| CARD11 | 12382772 | 1 | SVGHVRGPGPSVQHTTLNGDSLTSQLTLLGGNARGSFVHSVKP |
| | | | GSLAEKAGLREGHQLLLLEGCIRGERQSVPLDTCTKEEAHWTIQ |
| | | | RCSGPVTLHYKVNHEGYRK |
| CARD14 | 13129123 | 1 | RRPARRILSQVTMLAFQGDALLEQISVIGGNLTGIFIHRVTPGSA |
| | | | ADQMALRPGTQIVMVDYEASEPLFKAVLEDTTLEEAVGLLRRV |
| | | | DGFCCLSVKVNTDGYKR |
| | | | (SEQ ID NO:115) |
| CASK | 3087815 | 1 | TRVRLVQFQKNTDEPMGITLKMNELNHCIVARIMHGGMIHRQG |
| | | | TLHVGDEIREINGISVANQTVEQLQKMLREMRGSITFKIVPSYRT |
| | | | QS |
| Connector | 3930780 | 1 | LEQKAVLEQVQLDSPLGLEIHTTSNCQHFVSQVDTQVPTDSRLQ |
| Enhancer | | | IQPGDEVVQINEQVVVGWPRKNMVRELLREPAGLSLVLKKIPIP |
| Cytohesin | 3192908 | 1 | QRKLVTVEKQDNETFGFEIQSYRPQNQNACSSEMFTLICKIQEDS |
| Binding | | | PAHCAGLQAGDVLANINGVSTEGFTYKQVVDLIRSSGNLLTIET |
| Protein | | | LNG |
| Densin 180 | 16755892 | 1 | RCLIQTKGQRSMDGYPEQFCVRIEKNPGLGFSISGGISGQGNPFK |
| | | | PSDKGIFVTRVQPDGPASNLLQPGDKILQANGHSFVHMEHEKA |
| | | | VLLLKSFQNTVDLVIQRELTV |
| DLG1 | 475816 | 1 | IQVNGTDADYEYEEITLERGNSGLGFSIAGGTDNPHIGDDSSIFIT |
| | | | KIITGGAAAQDGRLRVNDCILQVNEVDVRDVTHSKAVEALKEA |
| | | | GSIVRLYVKRRN |
| DLG1 | 475816 | 2 | IQLIKGPKGLGFSIAGGVGNQHIPGDNSIYVTKIIEGGAAHKDGK |
| | | | LQIGDKLLAVNNVCLEEVTHEEAVTALKNTSDFVYLKVAKPTS |
| | | | MYMNDGN |
| DLG1 | 475816 | 3 | ILHRGSTGLGFNIVGGEDGEGIFISFILAGGPADLSGELRKGDRIIS |
| | | | VNSVDLRAASHEQAAAALKNAGQAVTIVAQYRPEEYSR |
| DLG2 | 12736552 | 1 | ISYVNGTEIEYEFEEITLERGNSGLGFSIAGGTDNPHIGDDPGIFIT |
| | | | KIIPGGAAAEDGRLRVNDCILRVNEVDVSEVSHSKAVEALKEAG |
| | | | SIVRLYVRRR |
| DLG2 | 12736552 | 2 | IPILETVVEIKLFKGPKGLGFSIAGGVGNQHIPGDNSIYVTKIIDGG |
| | | | AAQKDGRLQVGDRLLMVNNYSLEEVTHEEAVAILKNTSEVVYL |
| | | | KVGKPTTIVMTDPYGPPNSS |
| DLG2 | 12736552 | 3 | ILEGEPRKVVLHKGSTGLGFNIVGGEDGEGIFVSFILAGGPADLS |
| | | | GELQRGDQILSVNGIDLRGASHEQAAAALKGAGQTVTIIAQHQP |
| | | | EDYARFEAKIHDLNSS |
| DLG5 | 3650451 | 1 | GIPYVEEPRHVKVQKGSEPLGISIVSGEKGGIYVSKVTVGSIAHQ |
| | | | AGLEYGDQLLEFNGINLRSATEQQARLIIGQQCDTITILAQYNPH |
| | | | VHQLRNSSZLTD |
| DLG5 | 3650451 | 2 | GILAGDANKKTLEPRVVFIKKSQLELGVHLCGGNLHGVFVAEV |
| | | | EDDSPAKGPDGLVPGDLILEYGSLDVRNKTVEEVYVEMLKPRD |
| | | | GVRLKVQYRPEEFIVTD |
| DLG6, | 14647140 | 1 | PTSPEIQELRQMLQAPHFKALLSAHDTIAQKDFEPLLPPLPDNIPE |
| splice | | | SEEAMRIVCLVKNQQPLGATIKRHEMTGDILVARIIHGGLAERS |
| variant 1 | | | GLLYAGDKLVEVNGVSVEGLDPEQVIHILAMSRGTIMFKVVPVS |
| | | | DPPVNSS |
| DLG6, | AB053303 | 1 | PTSPEIQELRQMLQAPHFKGATIKRHEMTGDILVARIIHGGLAER |
| splice | | | SGLLYAGDKLVEVNGVSVEGLDPEQVIHILAMSRGTIMFKVVPV |
| variant 2 | | | SDPPVNSS |
| DVL1 | 2291005 | 1 | LNIVTVTLNMERHHFLGISIVGQSNDRGDGGIYIGSIMKGGAVA |
| | | | ADGRIEPGDMLLQVNDVNFENMSNDDAVRVLREIVSQTGPISLT |
| | | | VAKCW |
| DVL2 | 2291007 | 1 | LNIITVTLNMEKYNFLGISIVGQSNERGDGGIYIGSIMKGGAVAA |
| | | | DGRIEPGDMLLQVNDMNFENMSNDDAVRVLRDIVHKPGPIVLT |
| | | | VAKCWDPSPQNS |
| DVL3 | 6806886 | 1 | IITVTLNMEKYNFLGISIVGQSNERGDGGIYIGSIMKGGAVAADG |
| | | | RIEPGDMLLQVNEINFENMSNDDAVRVLREIVHKPGPITLTVAK |
| | | | CWDPSP |
| ELFIN 1 | 2957144 | 1 | LTTQQIDLQGPGPWGFRLVGRKDFEQPLAISRVTPGSKAALANL |
| | | | CIGDVITAIDGENTSNMTHLEAQNRIKGCTDNLTLTVARSEHKV |
| | | | WSPLVTNSS |
| ENIGMA | 561636 | 1 | IFMDSFKVVLEGPAPWGFRLQGGKDFNVPLSISRLTPGGKAAQA |
| | | | GVAVGDWVLSIDGENAGSLTHIEAQNKIRACGERLSLGLSRAQP |
| | | | V |
| ERBIN | 8923908 | 1 | QGHELAKQEIRVRVEKDPELGFSISGGVGGRGNPFRPDDDGIFV |
| | | | TRVQPEGPASKLLQPGDKIIQANGYSFINIEHGQAVSLLKTFQNT |
| | | | VELIIVREVSS |
| EZRIN | 3220018 | 1 | QMSADAAAGAPLPRLCCLEKGPNGYGFHLHGEKGKLGQYIRLV |
| Binding | | | EPGSPAEKAGLLAGDRLVEVNGENVEKETHQQVVSRIRAALNA |
| Protein 50 | | | VRLLVVDPETDEQLQKLGVQVREELLRAQEAPGQAEPPAAAEV |
| | | | QGAGNENEPREADKSHPEQRELRNSS |
| EZRIN | 3220018 | 2 | IQQRELRPRLCTMKKGPSGYGFNLHSDKSKPGQFIRSVDPDSPA |
| Binding | | | EASGLRAQDRIVEVNGVCMEGKQHGDVVSAIRAGGDETKLLV |
| Protein 50 | | | VDRETDEFFKNSS |
| FLJ00011 | 10440352 | 1 | KNPSGELKTVTLSKMKQSLGISISGGIESKVQPMVKIEKIFPGGA |
| | | | AFLSGALQAGFELVAVDGENLEQVTHQRAVDTIRRAYRNKARE |
| | | | PMELVVRVPGPSPRPSPSD |
| FLJ11215 | 11436365 | 1 | EGHSHPRVVELPKTEEGLGFNIMGGKEQNSPIYISRIIPGGIADRH |
| | | | GGLKRGDQLLSVNGVSVEGEHHEKAVELLKAAQGKVKLVVRY |
| | | | TPKVLEEME |
| FLJ12428 | BC012040 | 1 | PGAPYARKTFTIVGDAVGWGFVVRGSKPCHIQAVDPSGPAAAA |
| | | | GMKVCQFVVSVNGLNVLHVDYRTVSNLILTGPRTIVMEVMEEL |
| | | | EC |
| FLJ12615 | 10434209 | 1 | GQYGGETVKIVRIEKARDIPLGATVRNEMDSVIISRIVKGGAAEK |
| | | | SGLLHEGDEVLEINGIEIRGKDVNEVFDLLSDMHGTLTFVLIPSQ |
| | | | QIKPPPA |
| FLJ20075 | 7019938 | 1 | ILAHVKGIEKEVNVYKSEDSLGLTITDNGVGYAFIKRIKDGGVID |
| | | | SVKTICVGDHIESINGENIVGWRHYDVAKKLKELKKEELFTMKL |
| | | | IEPKKAFEI |
| FLJ21687 | 10437836 | 1 | KPSQASGHFSVELVRGYAGFGLTLGGGRDVAGDTPLAVRGLLK |
| | | | DGPAQRCGRLEVGDLVLHINGESTQGLTHAQAVERIRAGGPQL |
| | | | HLVIRRPLETHPGKPRGV |
| FLJ31349 | AK055911 | 1 | PVMSQCACLEEVHLPNIKPGEGLGMYIKSTYDGLHVITGTTENS |
| | | | PADRSQKIHAGDEVIQVNQQTVVGWQLKNLVKKLRENPTGVV |
| | | | LLLKKRPTGSFNFTPEFIVTD |
| FLJ32798 | AK057360 | 1 | LDDEEDSVKIIRLVKNREPLGATIKKDEQTGAIIVARIMRGGAAD |
| | | | RSGLIHVGDELREVNGIPVEDKRPEEIIQILAQSQGAITFKIIPGSK |
| | | | EETPSNSS |
| GoRASP1 | NM031899 | 1 | MGLGVSAEQPAGGAEGFHLHGVQENSPAQQAGLEPYFDFIITIG |
| | | | HSRLNKENDTLKALLKANVEKPVKLEVFNMKTMRVREVEVVP |
| | | | SNMWGGQGLLGASVRFCSFRRASE |
| GoRASP1 | NM031899 | 2 | RASEQVWHVLDVEPSSPAALAGLRPYTDYVVGSDQILQESEDFF |
| | | | TLIESHEGKPLKLMVYNSKSDSCREVTVTPNAAWGGEGSLGCGI |
| | | | GYGYLHRIPTQ |
| GoRASP2 | 13994253 | 1 | MGSSQSVEIPGGGTEGYHVLRVQENSPGHRAGLEPFFDFIVSING |
| | | | SRLNKDNDTLKDLLKANVEKPVKMLIYSSKTLELRETSVTPSNL |
| | | | WGGQGLLGVSIRFCSFDGANE |
| GoRASP2 | 13994253 | 2 | NENVWHVLEVESNSPAALAGLRPHSDYIIGADTVMNESEDLFSL |
| | | | IETHEAKPLKLYVYNTDTDNCREVIITPNSAWGGEGSLGCGIGY |
| | | | GYLHRIPTR |
| GRIP 1 | 4539083 | 1 | VVELMKKEGTTLGLTVSGGIDKDGKPRVSNLRQGGIAARSDQL |
| | | | DVGDYIKAVNGINLAKFRHDEIISLLKNVGERVVLEVEYE |
| GRIP 1 | 4539083 | 2 | RSSVIFRTVEVTLHKEGNTFGFVIRGGAHDDRNKSRPVVITCVRP |
| | | | GGPADREGTIKPGDRLLSVDGIRLLGTTHAEAMSILKQCGEAA |
| | | | LLIEYDVSVMDSVATASGNSS ( |
| GRIP 1 | 4539083 | 3 | HVATASGPLLVEVAKTPGASLGVALTTSMCCNKQVIVIDKIKSA |
| | | | SIADRCGALHVGDHILSIDGTSMEYCTLAEATQFLANTTDQVKL |
| | | | EILPHHQTRLALKGPNSS |
| GRIP 1 | 4539083 | 4 | HVATASGPLLVEVAKTPGASLGVALTTSMCCNKQVIVIDKIKSA |
| | | | SIADRCGALHVGDHILSIDGTSMEYCTLAEATQFLANTTDQVKL |
| | | | EILPHHQTRLALKGPNSS |
| GRIP 1 | 4539083 | 5 | AESVIPSSGTFHVKLPKKHNVELGITISSPSSRKPGDPLVISDIKKG |
| | | | SVAHRTGTLELGDKLLAIDNIRLDNCSMEDAVQILQQCEDLVKL |
| | | | KIRKDEDNSD |
| GRIP 1 | 4539083 | 6 | IYTVELKRYGGPLGITISGTEEPFDPIIISSLTKGGLAERTGAIHIGD |
| | | | RILAINSSSLKGKPLSEAIHLLQMAGETVTLKIKKQTDAQSA |
| GRIP 1 | 4539083 | 7 | IMSPTPVELHKVTLYKDSDMEDFGFSVADGLLEKGVYVKNIRP |
| | | | AGPGDLGGLKPYDRLLQVNHVRTRDFDCCLVVPLIAESGNKLD |
| | | | LVISRNPLA |
| GTPase | 2389008 | 1 | LSRGCETRELALPRDGQGRLGFEVDAEGFVTHVERFTFAETAGL |
| Activating | | | RPGARLLRVCGQTLPSLRPEAAAQLLRSAPKVCVTVLPPDESGR |
| Enzyme | | | PRNSS |
| Guanine | 6650765 | 1 | CSVMIFEVVEQAGAIILEDGQELDSWYVILNGTVEISHPDGKVEN |
| Exchange | | | LFMGNSFGITPTLDKQYMHGIVRTKVDDCQFVCIAQQDYWRIL |
| Factor | | | NHVEKNTHKVEEEGEIVMVHEFIVTD |
| HEMBA | 10436367 | 1 | LENVIAKSLLIKSNEGSYGFGLEDKNKVPIIKLVEKGSNAEMAG |
| 1000505 | | | MEVGKKIFAINGDLVFMRPFNEVDCFLKSCLNSRKPLRVLVSTK |
| | | | P |
| HEMBA | 10436367 | 2 | PRETVKIPDSADGLGFQIRGFGPSVVHAVGRGTVAAAAGLHPG |
| 1000505 | | | QCIIKVNGINVSKETHASVIAHVTACRKYRRPTKQDSIQNSS |
| HEMBA | 7022001 | 1 | EDFCYVFTVELERGPSGLGMGLIDGMHTHLGAPGLYIQTLLPGS |
| 1003117 | | | PAAADGRLSLGDRILEVNGSSLLGLGYLRAVDLIRHGGKKMRFL |
| | | | VAKSDVETAKKI |
| HSPC227 | 7106843 | 1 | NNELTQFLPRTITLKKPPGAQLGFNIRGGKASQLGIFISKVIPDSD |
| | | | AHRAGLQEGDQVLAVNDVDFQDIEHSKAVEILKTAREISMRVR |
| | | | FFPYNYHRQKE |
| HTRA3 | AY040094 | 1 | LTEFQDKQIKDWKKRFIGIRMRTITPSLVDELKASNPDFPEVSSGI |
| | | | YVQEVAPNSPSQRGGIQDGDIIVKVNGRPLVDSSELQEAVLTESP |
| | | | LLLEVRRGNDDLLFSNSS |
| | | | (SEQ ID NO:158) |
| HTRA4 | AL576444 | 1 | HKKYLGLQMLSLTVPLSEELKMHYPDFPDVSSGVYVCKVVEGT |
| | | | AAQSSGLRDHDVIVNINGKPITTTTDVVKALDSDSLSMAVLRGK |
| | | | DNLLLTVNSS |
| INADL | 2370148 | 1 | IWQIEYIDIERPSTGGLGFSVVALRSQNLGKVDIFVKDVQPGSVA |
| | | | DRDQRLKENDQILAINHTPLDQNISHQQAIALLQQTTGSLRLIVA |
| | | | REPVHTKSSTSSSE |
| INADL | 2370148 | 2 | LPETVCWGHVEEVELINDGSGLGFGIVGGKTSGVVVRTIVPGGL |
| | | | ADRDGRLQTGDHILKIGGTNVQGMTSEQVAQVLRNCGNSVRM |
| | | | LVARDPAGDISVTNSS |
| INADL | 2370148 | 3 | PGSDSSLFETYNVELVRKDGQSLGIRIVGYVGTSHTGEASGIYVK |
| | | | SIIPGSAAYHNGHIQVNDKIVAVDGVNIQGFANHDVVEVLRNAG |
| | | | QVVHLTLVRRKTSSSTSRIHRD |
| INADL | 2370148 | 4 | NSDDAELQKYSKLLPIHTLRLGVEVDSFDGHHYISSIVSGGPVDT |
| | | | LGLLQPEDELLEVNGMQLYGKSRREAVSFLKEVPPPFTLVCCRR |
| | | | LFDDEAS |
| INADL | 2370148 | 5 | LSSPEVKIVELVKDCKGLGFSILDYQDPLDPTRSVIVIRSLVADG |
| | | | VAERSGGLLPGDRLVSVNEYCLDNTSLAEAVEILKAVPPGLVHL |
| | | | GICKPLVEFIVTD |
| INADL | 2370148 | 6 | PNFSHWGPPRIVEIFREPNVSLGISIVVGQTVIKRLKNGEELKGIFI |
| | | | KQVLEDSPAGKTNALKTGDKILEVSGVDLQNASHSEAVEAIKN |
| | | | AGNPVVFIVQSLSSTPRVIPNVHNKANSS |
| INADL | 2370148 | 7 | PGELHIIELEKDKNGLGLSLAGNKDRSRMSIFVVGINPEGPAAAD |
| | | | GRMRIGDELLEINNQILYGRSHQNASAIIKTAPSKVKLVFIRNED |
| | | | AVNQMANSS |
| INADL | 2370148 | 8 | PATCPIVPGQEMIIEISKGRSGLGLSIVGGKDTPLNAIVIHEVYEE |
| | | | GAAARDGRLWAGDQILEVNGVDLRNSSHEEAITALRQTPQKVR |
| | | | LVVY |
| KIAA0147 | 1469875 | 1 | ILTLTILRQTGGLGISIAGGKGSTPYKGDDEGIFISRVSEEGPAAR |
| | | | AGVRVGDKLLEVNGVALQGAEHHEAVEALRGAGTAVQMRVW |
| | | | RERMVEPENAEFIVTD |
| KIAA0147 | 1469875 | 2 | PLRQRHVACLARSERGLGFSIAGGKGSTPYRAGDAGIFVSRIAE |
| | | | GGAAHRAGTLQVGDRVLSINGVDVTEARHDHAVSLLTAASPTI |
| | | | ALLLEREAGG |
| KIAA0147 | 1469875 | 3 | ILEGPYPVEEIRLPRAGGPLGLSIVGGSDHSSHPFGVQEPGVFISK |
| | | | VLPRGLAARSGLRVGDRILAVNGQDVRDATHQEAVSALLRPCL |
| | | | ELSLLVRRDPAEFIVTD |
| KIAA0147 | 1469875 | 4 | RELCIQKAPGERLGISIRGGARGHAGNPRDPTDEGIFISKVSPTGA |
| | | | AGRDGRLRVGLRLLEVNQQSLLGLTHGEAVQLLRSVGDTLTVL |
| | | | VCDGFEASTDAALEVS |
| KIAA0303 | 2224546 | 1 | PHQPIVIHSSGKNYGFTIRAIRVYVGDSDIYTVHHIVWNVEEGSP |
| | | | ACQAGLKAGDLITHINGEPVHGLVHTEVIELLLKSGNKVSITTTP |
| | | | F |
| KIAA0313 | 7657260 | 1 | HLRLLNIACAAKAKRRLMTLTKPSREAPLPFILLGGSEKGFGIFV |
| | | | DSVDSGSKATEAGLKRGDQILEVNGQNFENIQLSKAMEILRNNT |
| | | | HLSITVKTNLFVFKELLTRLSEEKRNGAPNSS |
| KIAA0316 | 6683123 | 1 | IPPAPRKVEMRRDPVLGFGFVAGSEKPVVVRSVTPGGPSEGKLIP |
| | | | GDQIVMINDEPVSAAPRERVIDLVRSCKESILLTVIQPYPSPKSEFI |
| | | | VTD |
| KIAA0340 | 2224620 | 1 | LNKRTTMPKDSGALLGLKVVGGKMTDLGRLGAFITKVKKGSL |
| | | | ADVVGHLRAGDEVLEWNGKPLPGATNEEVYNIILESKSEPQVEII |
| | | | VSRPIGDIPRIHRD |
| KIAA0380 | 2224700 | 1 | QRCVIIQKDQHGFGFTVSGDRIVLVQSVRPGGAAMKAGVKEGD |
| | | | RIIKVNGTMVTNSSHLEVVKLIKSGAYVALTLLGSS |
| KIAA0382 | 7662087 | 1 | ILVQRCVIIQKDDNGFGLTVSGDNPVFVQSVKEDGAAMRAGVQ |
| | | | TGDRIIKVNGTLVTHSNHLEVVKLIKSGSYVALTVQGRPPGNSS |
| KIAA0440 | 2662160 | 1 | SVEMTLRRNGLGQLGFHVNYEGIVADVEPYGYAWQAGLRQGS |
| | | | RLVEICKVAVATLSHEQMIDLLRTSVTVKVVIIPPHD |
| KIAA0545 | 14762850 | 1 | LKVMTSGWETVDMTLRRNGLGQLGFHVKYDGTVAEVEDYGF |
| | | | AWQAGLRQGSRLVEICKVAVVTLTHDQMIDLLRTSVTVKVVIIP |
| | | | PFEDGTPRRGW |
| | | | (SEQ ID NO:179) |
| KIAA0559 | 3043641 | 1 | HYIFPHARIKITRDSKDHTVSGNGLGIRIVGGKEIPGHSGEIGAYI |
| | | | AKILPGGSAEQTGKLMEGMQVLEWNGIPLTSKTYEEVQSIISQQ |
| | | | SGEAEICVRLDLNML |
| KIAA0561 | 3043645 | 1 | LCGSLRPPIVIHSSGKKYGFSLRAIRVYMGDSDVYTVHHVVWSV |
| | | | EDGSPAQEAGLRAGDLITHINGESVLGLVHMDVVELLLKSGNKI |
| | | | SLRTTALENTSIKVGNSS |
| KIAA0613 | 3327039 | 1 | SYSVTLTGPGPWGFRLQGGKDFNMPLTISRITPGSKAAQSQLSQ |
| | | | GDLVVAIDGVNTDTMTHLEAQNKIKSASYNLSLTLQKSKNSS |
| KIAA0751 | 12734165 | 1 | TLNEEHSHSDKHPVTWQPSKDGDRLIGRILLNKRLKDGSVPRDS |
| RIM2 | | | GAMLGLKVVGGKMTESGRLCAFITKVKKGSLADTVGHLRPGD |
| | | | EVLEWNGRLLQGATFEEVYNIILESKPEPQVELVVSRPIG |
| KIAA0807 | 3882334 | 1 | ISALGSMRPPIIIHRAGKKYGFTLRAIRVYMGDSDVYTVHHMVW |
| | | | HVEDGGPASEAGLRQGDLITHVNGEPVHGLVHTEVVELILKSGN |
| | | | KVAISTTPLENSS |
| KIAA0858 | 4240204 | 1 | FSDMRISINQTPGKSLDFGFTIKWDIPGIFVASVEAGSPAEFSQLQ |
| | | | VDDEIIAINNTKFSYNDSKEWEEAMAKAQETGHLVMDVRRYGK |
| | | | AGSPE |
| KIAA0902 | 4240292 | 1 | QSAHLEVIQLANIKPSEGLGMYIKSTYDGLHVITGTTENSPADRC |
| | | | KKIHAGDEVIQVNHQTVVGWQLKNLVNALREDPSGVILTLKKR |
| | | | PQSMLTSAPA |
| KIAA0967 | 4589577 | 1 | ILTQTLIPVRHTVKIDKDTLLQDYGFHISESLPLTVVAVTAGGSA |
| | | | HGKLFPGDQILQMNNEPAEDLSWERAVDILREAEDSLSITVVRC |
| | | | TSGVPKSSNSS |
| KIAA0973 | 4589589 | 1 | GLRSPITIQRSGKKYGFTLRAIRVYMGDTDVYSVHHIVWHVEEG |
| | | | GPAQEAGLCAGDLITHVNGEPVHGMVHPEVVELILKSGNKVAV |
| | | | TTTPFE |
| KIAA1095 | 5889526 | 1 | QGEETKSLTLVLHRDSGSLGFNIIGGRPSVDNHDGSSSEGIFVSKI |
| | | | VDSGPAAKEGGLQIHDRIIEVNGRDLSRATHDQAVEAFKTAKEP |
| | | | IVVQVLRRTPRTKMFTP |
| KIAA1095 | 5889526 | 2 | QEMDREELELEEVDLYRMNSQDKLGLTVCYRTDDEDDIGIYISE |
| | | | IDPNSIAAKDGRIREGDRIIQINGIEVQNREEAVALLTSEENKNFS |
| | | | LLIARPELQLD |
| KIAA1202 | 6330421 | 1 | RSFQYVPVQLQGGAPWGFTLKGGLEHCEPLTVSKIEDGGKAAL |
| | | | SQKMRTGDELVNINGTPLYGSRQEALILIKGSFRILKLIVRRRNA |
| | | | PVS |
| KIAA1222 | 6330610 | 1 | ILEKLELFPVELEKDEDGLGISIIGMGVGADAGLEKLGIFVKTVT |
| | | | EGGAAQRDGRIQVNDQIVEVDGISLVGVTQNFAATVLRNTKGN |
| | | | VRFVIGREKPGQVS |
| KIAA1284 | 6331369 | 1 | KDVNVYVNPKKLTVIKAKEQLKLLEVLVGIIHQTKWSWRRTGK |
| | | | QGDGERLVVHGLLPGGSAMKSGQVLIGDVLVAVNDVDVTTENI |
| | | | ERVLSCIPGPMQVKLTFENAYDVKRET |
| KIAA1389 | 7243158 | 1 | TRGCETVEMTLRRNGLGQLGFHVNFEGIVADVEPFGFAWKAGL |
| | | | RQGSRLVEICKVAVATLTHEQMIDLLRTSVTVKVVIIQPHDDGSP |
| | | | RR |
| KIAA1415 | 7243210 | 1 | VENILAKRLLILPQEEDYGFDIEEKNKAVVVKSVQRGSLAEVAG |
| | | | LQVGRKIYSINEDLVFLRPFSEVESILNQSFCSRRPLRLLVATKAK |
| | | | EIIKIP |
| | | | (SEQ ID NO:195) |
| KIAA1526 | 5817166 | 1 | PDSAGPGEVRLVSLRRAKAHEGLGFSIRGGSEHGVGIYVSLVEP |
| | | | GSLAEKEGLRVGDQILRVNDKSLARVTHAEAVKALKGSKKLVL |
| | | | SVYSAGRIPGGYVTNHIEFIVTD |
| KIAA1526 | 5817166 | 2 | LQGGDEKKVNLVLGDGRSLGLTIRGGAEYGLGIYITGVDPGSEA |
| | | | EGSGLKVGDQILEVNWRSFLNILHDEAVRLLKSSRHLILTVKDV |
| | | | GRLPHARTTVDEEFIVTD |
| KIAA1526 | 5817166 | 3 | WTSGAHVHSGPCEEKCGHPGHRQPLPRIVTIQRGGSAHNCGQL |
| | | | KVGHVILEVNGLTLRGKEHREAARIIAEAFKTKDRDYIDFLDSL |
| KIAA1620 | 10047316 | 1 | ELRRAELVEIIVETEAQTGVSGINVAGGGKEGIFVRELREDSPAA |
| | | | RSLSLQEGDQLLSARVFFENFKYEDALRLLQCAEPYKVSFCLKR |
| | | | TVPTGDLALRP |
| KIAA1634 | 10047344 | 1 | PSQLKGVLVRASLKKSTMGFGFTIIGGDRPDEFLQVKNVLKDGP |
| | | | AAQDGKIAPGDVIVDINGNCVLGHTHADVVQMFQLVPVNQYV |
| | | | NLTLCRGYPLPDDSED |
| KIAA1634 | 10047344 | 2 | ASSGSSQPELVTIPLIKGPKGFGFAIADSPTGQKVKMILDSQWCQ |
| | | | GLQKGDIIKEIYHQNVQNLTHLQVVEVLKQFPVGADVPLLILRG |
| | | | GPPSPTKTAKM |
| KIAA1634 | 10047344 | 3 | LYEDKPPLTNTFLISNPRTTADPRILYEDKPPNTKDLDVFLRKQE |
| | | | SGFGFRVLGGDGPDQSIYIGAIIPLGAAEKDGRLRAADELMCIDG |
| | | | IPVKGKSHKQVLDLMTTAARNGHVLLTVRRKIFYGEKQPEDDS |
| | | | GSPGIHRELT |
| KIAA1634 | 10047344 | 4 | PAPQEPYDVVLQRKENEGFGFVILTSKNKPPPGVIPHKIGRVIEG |
| | | | SPADRCGKLKVGDHISAVNGQSIVELSHDNIVQLIKDAGVTVTL |
| | | | TVIAEEEHHGPPS |
| KIAA1634 | 10047344 | 5 | QNLGCYPVELERGPRGFGFSLRGGKEYNMGLFILRLAEDGPAIK |
| | | | DGRIHVGDQIVEINGEPTQGITHTRAIELIQAGGNKVLLLLRPGT |
| | | | GLIPDHGLA |
| KIAA1719 | 1267982 | 0 | ITVVELIKKEGSTLGLTISGGTDKDGKPRVSNLRPGGLAARSDLL |
| | | | NIGDYIRSVNGIHLTRLRHDEIITLLKNVGERVVLEVEY |
| KIAA1719 | 1267982 | 1 | ILDVSLYKEGNSFGFVLRGGAHEDGHKSRPLVLTYVRPGGPAD |
| | | | REGSLKVGDRLLSVDGIPLHGASHATALATLRQCSHEALFQVEY |
| | | | DVATP |
| KIAA1719 | 1267982 | 2 | IHTVANASGPLMVEIVKTPGSALGISLTTTSLRNKSVITIDRIKPAS |
| | | | VVDRSGALHPGDHILSIDGTSMEHCSLLEATKLLASISEKVRLEIL |
| | | | PVPQSQRPL |
| KIAA1719 | 1267982 | 3 | IQIVHTETTEVVLCGDPLSGFGLQLQGGIFATETLSSPPLVCFIEPD |
| | | | SPAERCGLLQVGDRVLSINGIATEDGTMEEANQLLRDAALAHK |
| | | | VVLEVEFDVAESV |
| KIAA1719 | 1267982 | 4 | IQFDVAESVIPSSGTFHVKLPKKRSVELGITISSASRKRGEPLIISDI |
| | | | KKGSVAHRTGTLEPGDKLLAIDNIRLDNCPMEDAVQILRQCEDL |
| | | | VKLKIRKDEDN |
| KIAA1719 | 1267982 | 5 | IQTTGAVSYTVELKRYGGPLGITISGTEEPFDPIVISGLTKRGLAE |
| | | | RTGAIHVGDRILAINNVSLKGRPLSEAIHLLQVAGETVTLKIKKQ |
| | | | LDR ) |
| KIAA1719 | 1267982 | 6 | ILEMEELLLPTPLEMHKVTLHKDPMRHDFGFSVSDGLLEKGVY |
| | | | VHTVRPDGPAHRGGLQPFDRVLQVNHVRTRDFDCCLAVPLLAE |
| | | | AGDVLELIISRKPHTAHSS |
| LIM | 12734250 | 1 | MALTVDVAGPAPWGFRITGGRDFHTPIMVTKVAERGKAKDAD |
| Mystique | | | LRPGDIIAINGESAEGMLHAEAQSKIRQSPSPLRLQLDRSQATS |
| | | | PGQT |
| LIM Protein | 3108092 | 1 | SNYSVSLVGPAPWGFRLQGGKDFNMPLTISSLKDGGKAAQANV |
| | | | RIGDVVLSIDGINAQGMTHLEAQNKIKGCTGSLNMYLQRAS |
| LIMK1 | 4587498 | 1 | TLVEHSKLYCGHCYYQTVVTPVIEQILPDSPGSHLPHTVTLVSIP |
| | | | ASSHGKRGLSVSIDPPHGPPGCGTEHSHTVRVQGVDPGCMSPDV |
| | | | KNSIHVGDRILEINGTPIRNVPLDEIDLLIQETSRLLQLTLEHD |
| LIMK2 | 1805593 | 1 | PYSVTLISMPATTEGRRGFSVSVESACSNYATTVQVKEVNRMHI |
| | | | SPNNRNAIHPGDRILEINGTPVRTLRVEEVEDAISQTSQTLQLLIE |
| | | | HD |
| LIM-RIL | 1085021 | 1 | IHSVTLRGPSPWGFRLVGRDFSAPLTISRVHAGSKASLAALCPGD |
| | | | LIQAINGESTELMTHLEAQNRIKGCHDHLTLSVSRPE |
| LU-1 | U52111 | 1 | VCYRTDDEEDLGIYVGEVNPNSIAAKDGRIREGDRIIQINGVDVQ |
| | | | NREEAVAILSQEENTNISLLVARPESQLA |
| MAGI1 | 3370997 | 1 | PSELKGKFIHTKLRKSSRGFGFTVVGGDEPDEFLQIKSLVLDGPA |
| | | | ALDGKMETGDVIVSVNDTCVLGHTHAQVVKIFQSIPIGASVDLE |
| | | | LCRGYPLPFDPDGIHRD |
| MAGI1 | 3370997 | 2 | PATQPELITVHIVKGPMGFGFTIADSPGGGGQRVKQIVDSPRCRG |
| | | | LKEGDLIVEVNKKNVQALTHNQVVDMLVECPKGS |
| | | | EVTLLVQRGGNSSZ |
| MAGI1 | 3370997 | 3 | QATQEQDFYTVELERGAKGFGFSLRGGREYNMDLYVLRLAED |
| | | | GPAERCGKMRIGDEILEINGETTKNMKHSRAIELIKNGGRRVRLF |
| | | | LKRG |
| MAGI1 | 3370997 | 4 | PGVVSTVVQPYDVEIRRGENEGFGFVIVSSVSRPEAGTTFAGNA |
| | | | CVAMPHKIGRIIEGSPADRCGKLKVGDRILAVNGCSITNKSHSDI |
| | | | VNLIKEAGNTVTLRIIPGDESSNAEFIVTD |
| MAGI1 | 3370997 | 5 | PDYQEQDIFLWRKETGFGFRILGGNEPGEPIYIGHIVPLGAADTD |
| | | | GRLRSGDELICVDGTPVIGKSHQLVVQLMQQAAKQGHVNLTVR |
| | | | RKVVFAVPKTENSS |
| MGC5395 | BC012477 | 1 | PAKMEKEETTRELLLPNWQGSGSHGLTIAQRDDGVFVQEVTQN |
| | | | SPAARTGVVKEGDQIVGATIYFDNLQSGEVTQLLNTMGHHTVG |
| | | | LKLHRKGDRSPNSS |
| MINT1 | 2625024 | 1 | SENCKdVFIEKQKGEILGVVIVESGWGSILPTVIIANMMHGGPAE |
| | | | KSGKLNIGDQIMSINGTSLVGLPLSTCQSIIKGLKNQSRVKLNIVR |
| | | | CPPVNSS |
| MINT1 | 2625024 | 2 | LRCPPVTTVLIRRPDLRYQLGFSVQNGIICSLMRGGIAERGGVRV |
| | | | GHRIIEINGQSVVATPHEKIVHILSNAVGEIHMKTMPAAMYRLL |
| | | | NSS |
| MINT3 | 3169808 | 1 | HNGDLDHFSNSDNCREVHLEKRRGEGLGVALVESGWGSLLPTA |
| | | | VIANLLHGGPAERSGALSIGDRLTAINGTSLVGLPLAACQAAVR |
| | | | ETKSQTSVTLSIVHCPPVT |
| MINT3 | 3169808 | 2 | PVTTAIIHRPHAREQLGFCVEDGIICSLLRGGIAERGGIRVGHRIIE |
| | | | INGQSVVATPHARIIELLTEAYGEVHIKTMPAATYRLLTG NSS |
| MPP1 | 189785 | 1 | RKVRLIQFEKVTEEPMGITLKLNEKQSCTVARILHGGMIHPQGS |
| | | | LHVGDEILEINGTNVTNHSVDQLQKAMKETKGMISLKVIPNQ |
| MPP2 | 939884 | 1 | PVPPDAVRMVGIRKTAGEHLGVTFRVEGGELVIARILHGGMVA |
| | | | QQGLLHVGDIIKEVNGQPVGSDPRALQELLRNASGSVILKILPNY |
| | | | Q |
| MPP3 | 1022812 | 1 | NIDEDFDEESVKIVRLVKNKEPLGATIRRDEHSGAVVVARIMRG |
| | | | GAADRSGLVHVGDELREVNGIAVLHKRPDEISQILAQSQGSITLK |
| | | | IIPATQEEDR |
| MUPP1 | 2104784 | 1 | QGRHVEVFELLKPPSGGLGFSVVGLRSENRGELGIFVQEIQEGSV |
| | | | AHRDGRLKETDQILAINGQALDQTITHQQAISILQKAKDTVQLVI |
| | | | ARGSLPQLV |
| MUPP1 | 2104784 | 2 | PVHWQHMETIELVNDGSGLGFGIIGGKATGVIVKTILPGGVADQ |
| | | | HGRLCSGDHILKIGDTDLAGMSSEQVAQVLRQCGNRVKLMIAR |
| | | | GAIEERTAPT |
| MUPP1 | 2104784 | 3 | QESETFDVELTKNVQGLGITIAGYIGDKKLEPSGIFVKSITKSSAV |
| | | | EHDGRIQIGDQIIAVDGTNLQGFTNQQAVEVLRHTGQTVLLTLM |
| | | | RRGMKQEA |
| MUPP1 | 2104784 | 4 | LNYEIVVAHVSKFSENSGLGISLEATVGHHFIRSVLPEGPVGHSG |
| | | | KLFSGDELLEVNGITLLGENHQDVVNILKELPIEVTMVCCRRTVP |
| | | | PT |
| MUPP1 | 2104784 | 5 | WEAGIQHIELEKGSKGLGFSILDYQDPIDPASTVIIIRSLVPGGIAE |
| | | | KDGRLLPGDRLMFVNDVNLENSSLEEAVEALKGAPSGTVRIGV |
| | | | AKPLPLSPEENSS |
| MUPP1 | 2104784 | 6 | RNVSKESFERTINIAKGNSSLGMTVSANKDGLGMIVRSIIHGGAI |
| | | | SRDGRIAIGDCILSINEESTISVTNAQARAMLRRHSLIGPDIKITYV |
| | | | PAEHLEE |
| MUPP1 | 2104784 | 7 | LNWNQPRRVELWREPSKSLGISIVGGRGMGSRLSNGEVMRGIFI |
| | | | KHVLEDSPAGKNGTLKPGDRIVEVDGMDLRDASHEQAVEAIRK |
| | | | AGNPVVFMVQSIINRPRKSPLPSLL |
| MUPP1 | 2104784 | 8 | LTGELHMIELEKGHSGLGLSLAGNKDRSRMSVFIVGIDPNGAAG |
| | | | KDGRLQIADELLEINGQILYGRSHQNASSIIKCAPSKVKIIFIRNKD |
| | | | AVNQ |
| MUPP1 | 2104784 | 9 | LSSFKNVQHLELPKDQGGLGIAISEEDTLSGVIIKSLTEHGVAAT |
| | | | DGRLKVGDQILAVDDEIVVGYPIEKFISLLKTAKMTVKLTIHAEN |
| | | | PDSQ |
| MUPP1 | 2104784 | 10 | LPGCETTIEISKGRTGLGLSIVGGSDTLLGAIIIHEVYEEGAACKD |
| | | | GRLWAGDQILEVNGIDLRKATHDEAINVLRQTPQRVRLTLYRD |
| | | | EAPYKE |
| MUPP1 | 2104784 | 11 | KEEEVCDTLTIELQKKPGKGLGLSIVGKRNDTGVFVSDIVKGGIA |
| | | | DADGRLMQGDQILMVNGEDVRNATQEAVAALLKCSLGTVTLE |
| | | | VGRIKAGPFHS |
| MUPP1 | 2104784 | 12 | LQGLRTVEMKKGPTDSLGISIAGGVGSPLGDVPIFIAMMHPTGV |
| | | | AAQTQKLRVGDRIVTICGTSTEGMTHTQAVNLLKNASGSIEMQ |
| | | | VVAGGDVSV |
| MUPP1 | 2104784 | 13 | LGPPQCKSITLERGPDGLGFSIVGGYGSPHGDLPIYVKTVFAKGA |
| | | | ASEDGRLKRGDQIIAVNGQSLEGVTHEEAVAILKRTKGTVTLMV |
| | | | LS |
| NeDLG | 10863920 | 1 | IQYEEIVLERGNSGLGFSIAGGIDNPHVPDDPGIFITKIIPGGAAAM |
| | | | DGRLGVNDCVLRVNEVEVSEVVHSRAVEALKEAGPVVRLVVR |
| | | | RRQN |
| NeDLG | 10863920 | 2 | ITLLKGPKGLGFSIAGGIGNQHIPGDNSIYITKIIEGGAAQKDGRL |
| | | | QIGDRLLAVNNTNLQDVRHEEAVASLKNTSDMVYLKVAKPGSL |
| | | | E |
| NeDLG | 10863920 | 3 | ILLHKGSTGLGFNIVGGEDGEGIFVSFILAGGPADLSGELRRGDRI |
| | | | LSVNGVNLRNATHEQAAAALKRAGQSVTIVAQYRPEEYSRFES |
| | | | KIHDLREQMMNSSMSSGSGSLRTSEKRSLE |
| Neurabin II | AJ401189 | 1 | CVERLELFPVELEKDSEGLGISIIGMGAGADMGLEKLGIFVKTVT |
| | | | EGGAAHRDGRIQVNDLLVEVDGTSLVGVTQSFAASVLRNTKGR |
| | | | VRFMIGRERPGEQSEVAQRIHRD |
| | | | (SEQ ID NO:247) |
| NOS1 | 642525 | 1 | IQPNVISVRLFKRKVGGLGFLVKERVSKPPVIISDLIRGGAAEQSG |
| | | | LIQAGDIILAVNGRPLVDLSYDSALEVLRGIASETHVVLILRGP |
| novel PDZ | 7228177 | 1 | QANSDESDIIHSVRVEKSPAGRLGFSVRGGSEHGLGIFVSKVEEG |
| gene | | | SSAERAGLCVGDKITEVNGLSLESTTMGSAVKVLTSSSRLHMM |
| | | | VRRMGRVPGIKFSKEKNSS |
| novel PDZ | 7228177 | 2 | PSDTSSEDGVRRIVHLYTTSDDFCLGFNIRGGKEFGLGIYVSKVD |
| gene | | | HGGLAEENGIKVGDQVLAANGVRFDDISHSQAVEVLKGQTHIM |
| | | | LTIKETGRYPAYKEMNSS |
| Novel | 1621243 | 1 | KIKKFLTESHDRQAKGKAITKKKYIGIRMMSLTSSKAKELKDRH |
| Serine | | | RDFPDVISGAYIIEVIPDTPAEAGGLKENDVIISINGQSVVSANDV |
| Protease | | | SDVIKRESTLNMVVRRGNEDIMITV |
| Numb | AK056823 | 1 | PDGEITSIKINRVDPSESLSIRLVGGSETPLVHIIIQHIYRDGVIARD |
| Binding | | | GRLLPGDIILKVNGMDISNVPHNYAVRLLRQPCQVLWLTVMRE |
| Protein | | | QKFRSRNSS |
| Numb | AK056823 | 2 | HRPRDDSFHVILNKSSPEEQLGIKLVRKVDEPGVFIFNVLDGGVA |
| Binding | | | YRHGQLEENDRVLAINGHDLRYGSPESAAHLIQASERRVHLVVS |
| Protein | | | RQVRQRSPENSS |
| Numb | AK056823 | 3 | PTITCHEKVVNIQKDPGESLGMTVAGGASHREWDLPIYVISVEP |
| Binding | | | GGVISRDGRIKTGDILLNVDGVELTEVSRSEAVALLKRTSSSIVL |
| Protein | | | KALEVKEYEPQEFIV |
| Outer | 7023825 | 1 | LLTEEEINLTRGPSGLGFNIVGGTDQQYVSNDSGIYVSRIKENGA |
| Membrane | | | AALDGRLQEGDKILSVNGQDLKNLLHQDAVDLFRNAGYAVSL |
| | | | RVQHRLQVQNGIHS |
| p55T | 12733367 | 1 | PVDAIRILGIHKRAGEPLGVTFRVENNDLVIARILHGGMIDRQGL |
| | | | LHVGDIIKEVNGHEVGNNPKELQELLKNISGSVTLKILPSYRDTIT |
| | | | PQQ |
| PAR3 | 8037914 | 1 | PNFSLDDMVKLVEVPNDGGPLGIHVVPFSARGGRTLGLLVKRL |
| | | | EKGGKAEHENLFRENDCIVRINDGDLRNRRFEQAQHMFRQAMR |
| | | | TPIIWFHVVPAANKEQYEQ |
| PAR3 | 8037914 | 2 | GKRLNIQLKKGTEGLGFSITSRDVTIGGSAPIYVKNILPRGAAIQD |
| | | | GRLKAGDRLIEVNGVDLVGKSQEEVVSLLRSTKMEGTVSLLVF |
| | | | RQEDA |
| PAR3 | 8037914 | 3 | PREFLTFEVPLNDSGSAGLGVSVKGNRSKENHADLGIFVKSIING |
| | | | GAASKDGRLRVNDQLIAVNGESLLGKTNQDAMETLRRSMSTEG |
| | | | NKRGMIQLIVASRISKCNELKSNSS |
| PAR3-like | AF428250 | 1 | PRTKDTLSDMTRTVEISGEGGPLGIHVVPFFSSLSGRILGLFIRGIE |
| | | | DNSRSKREGLFHENECIVKINNVDLVDKTFAQAQDVFRQAMKS |
| | | | PSVLLHVLPPQNR |
| PAR3-like | AF428250 | 2 | SNKNAKKIKIDLKKGPEGLGFTVVTRDSSIHGPGPIFVKNILPKG |
| | | | AAIKDGRLQSGDRILEVNGRDVTGRTQEELVAMLRSTKQGETA |
| | | | SLVIARQEGH |
| PAR3-like | AF428250 | 3 | ITSEQLTFEIPLNDSGSAGLGVSLKGNKSRETGTDLGIFIKSIIHGG |
| | | | AAFKDGRLRMNDQLIAVNGESLLGKSNHEAMETLRRSMSMEG |
| | | | NIRGMIQLVILRRPERP |
| PAR6 | 2613011 | 1 | PETHRRVRLHKHGSDRPLGFYIRDGMSVRVAPQGLERVPGIFISR |
| | | | LVRGGLAESTGLLAVSDEILEVNGIEVAGKTLDQVTDMMVANS |
| | | | HNLIVTVKPANQRNNVNSS |
| PAR6 | 13537116 | 1 | PVSSIIDVDILPETHRRVRLYKYGTEKPLGFYIRDGSSVRVTPHGL |
| BETA | | | EKVPGIFISRLVPGGLAQSTGLLAVNDEVLEVNGIEVSGKSLDQV |
| | | | TDMMIANSRNLIITVRPANQRNNRIHRD |
| PAR6 | 13537118 | 1 | IDVDLVPETHRRVRLHRHGCEKPLGFYIRDGASVRVTPHGLEKV |
| GAMMA | | | PGIFISRMVPGGLAESTGLLAVNDEVLEVNGIEVAGKTLDQVTD |
| | | | MMIANSHNLIVTVKPANQRNNVV |
| PDZ-73 | 5031978 | 1 | RSRKLKEVRLDRLHPEGLGLSVRGGLEFGCGLFISHLIKGGQAD |
| | | | SVGLQVGDEIVRINGYSISSCTHEEVINLIRTKKTVSIKVRHIGLIP |
| | | | VKSSPDEFH |
| PDZ-73 | 5031978 | 2 | IPGNRENKEKKVFISLVGSRGLGCSISSGPIQKPGIFISHVKPGSLS |
| | | | AEVGLEIGDQIVEVNGVDFSNLDHKEAVNVLKSSRSLTISIVAAA |
| | | | GRELFMTDEF |
| PDZ-73 | 5031978 | 3 | PEQIMGKDVRLLRIKKEGSLDLALEGGVDSPIGKVVVSAVYERG |
| | | | AAERHGGIVKGDEIMAINGKIVTDYTLAEADAALQKAWNQGG |
| | | | DWIDLVVAVCPPKEYDD |
| PDZK1 | 2944188 | 1 | LTSTFNPRECKLSKQEGQNYGFFLRIEKDTEGHLVRVVEKCSPA |
| | | | EKAGLQDGDRVLRINGVFVDKEEHMQVVDLVRKSGNSVTLLV |
| | | | LDGDSYEKAGSPGIHRD |
| PDZK1 | 2944188 | 2 | RLCYLVKEGGSYGFSLKTVQGKKGVYMTDITPQGVAMRAGVL |
| | | | ADDHLIEVNGENVEDASHEEVVEKVKKSGSRVMFLLVDKETDK |
| | | | REFIVTD |
| PDZK1 | 2944188 | 3 | QFKRETASLKLLPHQPRIVEMKKGSNGYGFYLRAGSEQKGQIIK |
| | | | DIDSGSPAEEAGLKNNDLVVAVNGESVETLDHDSVVEMIRKGG |
| | | | DQTSLLVVDKETDNMYRLAEFIVTD |
| PDZK1 | 2944188 | 4 | PDTTEEVDHKPKLCRLAKGENGYGFHLNAIRGLPGSFIKEVQKG |
| | | | GPADLAGLEDEDVIIEVNGVNVLDEPYEKVVDRIQSSGKNVTLL |
| | | | VZGKNSS |
| PICK1 | 4678411 | 1 | PTVPGKVTLQKDAQNLIGISIGGGAQYCPCLYIVQVFDNTPAAL |
| | | | DGTVAAGDEITGVNGRSIKGKTKVEVAKMIQEVKGEVTIHYNK |
| | | | LQ |
| PIST | 98374330 | 1 | SQGVGPIRKVLLLKEDHEGLGISITGGKEHGVPILISEIHPGQPAD |
| | | | RCGGLHVGDAILAVNGVNLRDTKHKEAVTILSQQRGEIEFEVVY |
| | | | VAPEVDSD |
| prIL16 | 1478492 | 1 | IHVTILHKEEGAGLGFSLAGGADLENKVITVHRVFPNGLASQEG |
| | | | TIQKGNEVLSINGKSLKGTTHHDALAILRQAREPRQAVIVTRKLT |
| | | | PEEFIVTD |
| prIL16 | 1478492 | 2 | TAEATVCTVTLEKMSAGLGFSLEGGKGSLHGDKPLTINRIFKGA |
| | | | ASEQSETVQPGDEILQLGGTAMQGLTRFEAWNIIKALPDGPVTIV |
| | | | IRRKSLQSK |
| PSAP | 6409315 | | IREAKYSGVLSSIGKIFKEEGLLGFFVGLIPHLLGDVVFLWGCNL |
| | | | LAHFINAYLVDDSVSDTPGGLGNDQNPGSQFSQALAIRSYTKFV |
| | | | MGIAVSMLTYPFLLVGDLMAVNNCGLQAGLPPYSPVFKSWIHC |
| | | | WKYLSVQGQLFRGSSLLFRRVSSGSCFALE |
| PSD95 | 3318652 | 1 | LEYEeITLERGNSGLGFSIAGGTDNPHIGDDPSIFITKIIPGGAAAQ |
| | | | DGRLRVNDSILFVNEVDVREVTHSAAVEALKEAGSIVRLYVMR |
| | | | RKPAENSS |
| PSD95 | 3318652 | 2 | HVMRRKPPAEKVMEIKLIKGPKGLGFSIAGGVGNQHIPGDNSIY |
| | | | VTKIIEGGAAHKDGRLQIGDKILAVNSVGLEDVMHEDAVAALK |
| | | | NTYDVVYLKVAKPSNAYLLEFIVTD |
| PSD95 | 3318652 | 3 | RERHTPRTEANCDHRGSTGLGFNIVGGEDGEGILSPLSWPGALQ |
| | | | TSVGSCGRGTRSCRSTVWTSEMPAMSRLPLP |
| PTN-3 | 179912 | 1 | QNDNGDSYLVLIRITPDEDGKFGFNLKGGVDQKMPLVVSRINPE |
| | | | SPADTCIPKLNEGDQIVLINGRDISEHTHDQVVMFIKASRESHSR |
| | | | ELALVIRRRAVRS |
| PTN-4 | 190747 | 1 | IRMKPDENGRFGFNVKGGYDQKMPVIVSRVAPGTPADLCVPRL |
| | | | NEGDQVVLINGRDIAEHTHDQVVLFIKASCERHSGELMLLVRPN |
| | | | A |
| PTPL1 | 515030 | 1 | PEREITLVNLKKDAKYGLGFQIIGGEKMGRLDLGIFISSVAPGGP |
| | | | ADFHGCLKPGDRLISVNSVSLEGVSHHAAIEILQNAPEDVTLVIS |
| | | | QPKEKISKVPSTPVHL |
| PTPL1 | 515030 | 2 | GDIFEVELAKNDNSLGISVTGGVNTSVRHGGIYVKAVIPQGAAE |
| | | | SDGRIHKGDRVLAVNGVSLEGATHKQAVETLRNTGQVVHLLLE |
| | | | KGQSPTSK |
| PTPL1 | 515030 | 3 | TEENTFEVKLFKNSSGLGFSFSREDNLIPEQINASIVRVKKLFAGQ |
| | | | PAAESGKIDVGDVILKVNGASLKGLSQQEVISALRGTAPEVFLLL |
| | | | CRPPPGVLPEIDT |
| PTPL1 | 515030 | 4 | ELEVELLITLIKSEKASLGFTVTKGNQRIGCYVHDVIQDPAKSDG |
| | | | RLKPGDRLIKVNDTDVTNMTHTDAVNLLRAASKTVRLVIGRVL |
| | | | ELPRIPMLPH |
| PTPL1 | 515030 | 5 | MLPHLLPDITLTCNKEELGFSLCGGHDSLYQVVYISDINPRSVAA |
| | | | IEGNLQLLDVIHYVNGVSTQGMTLEEVNRALDMSLPSLVLKAT |
| | | | RNDLPV |
| RGS12 | 3290015 | 1 | RPSPPRVRSVEVARGRAGYGFTLSGQAPCVLSCVMRGSPADFV |
| | | | GLRAGDQILAVNEINVKKASHEDVVKLIGKCSGVLHMVIAEGV |
| | | | GRFESCSNSS |
| RGS3 | 18644735 | 1 | LCSERRYRQITIPRGKDGFGFTICCDSPVRVQAVDSGGPAERAGL |
| | | | QQLDTVLQLNERPVEHWKCVELAHEIRSCPEIILLVWRMVPQV |
| | | | KPGIHRD |
| Rho-GAP | NM020824 | 1 | SEDETFSWPGPKTVTLKRTSQGFGFTLRHFIVYPPESAIQFSYKD |
| 10 | | | EENGNRGGKQRNRLEPMDTIFVKQVKEGGPAFEAGLCTGDRIIK |
| | | | VNGESVIGKTYSQVIALIQNSDTTLELSVMPKDED |
| Rhophilin- | 14279408 | 1 | SAKNRWRLVGPVHLTRGEGGFGLTLRGDSPVLIAAVIPGSQAAA |
| like | | | AGLKEGDYIVSVNGQPCRWWRHAEVVTELKAAGEAGASLQVV |
| | | | SLLPSSRLPSI |
| Serine | 2738914 | 1 | RGEKKNSSSGISGSQRRYIGVMMLTLSPSILAELQLREPSFPDVQ |
| Protease | | | HGVLIHKVILGSPAHRAGLRPGDVILAIGEQMVQNAEDVYEAVR |
| | | | TQSQLAVQIRRGRETLTLYVNSS |
| Shank 2 | 6049185 | 1 | LEEKTVVLQKKDNEGFGFVLRGAKADTPIEEFTPTPAFPALQYL |
| | | | ESVDEGGVAWQAGLRTGDFLIEVNNENVVKVGHRQVVNMIRQ |
| | | | GGNHLVLKVVTVTRNLDPDDNSS |
| Shank 3 | * | 1 | SDYVIDDKVAVLQKRDHEGFGFVLRGAKAETPIEEFTPTPAFPA |
| | | | LQYLESVDVEGVAWRAGLRTGDFLIEVNGVNVVKVGHKQVVA |
| | | | LIRQGGNRLVMKVVSVTRKPEEDG |
| Shroom | 18652858 | 1 | ISNTATKGRYIYLEAFLEGGAPWGFTLKGGLEHGEPLIISKVEEG |
| | | | GKADTLSSKLQAGDEVVHINEVTLSSSRKEAVSLVKGSYKTLRL |
| | | | VVRRDVCTDPGHAD |
| Similar to | 14286261 | 1 | MGLGVSAEQPAGGAEGFHLHGVQENSPAQQAGLEPYFDFIITIG |
| GRASP65 | | | HSRLNKENDTLKALLKANVEKPVKLEVFNMKTMRVREVEVVP |
| | | | SNMWGGQGLLGASVRFCSFRRASE |
| Similar to | 14286261 | 2 | RASEQVWHVLDVEPSSPAALAGLRPYTDYVVGSDQILQESEDFF |
| GRASP65 | | | TLIESHEGKPLKLMVYNSKSDSCRESGMWHWLWVSTPDPNSAP |
| | | | QLPQEATWHPTTFCSTTWCPTT |
| Similar to | BC036755 | 1 | IQPLSLPEGEITTIEIHRSNPYIQLGISIVGGNETPLINIVIQEVYRDG |
| Ligand of | | | VIARDGRLLAGDQILQVNNYNISNVSHNYARAVLSQPCNTLHLT |
| Numb px2 | | | VLRERRFGNRAH |
| Similar to | BC036755 | 2 | SNSPREEIFQVALHKRDSGEQLGIKLVRRTDEPGVFILDLLEGGL |
| Ligand of | | | AAQDGRLSSNDRVLAINGHDLKYGTPELAAQIIQASGERVNLTI |
| Numb px2 | | | ARPGKPQPG |
| Similar to | BC036755 | 3 | QCVTCQEKHITVKKEPHESLGMTVAGGRGSKSGELPIFVTSVPP |
| Ligand of | | | HGCLARDGRIKRGDVLLNINGIDLTNLSHSEAVAMLKASAASPA |
| Numb px2 | | | VALKALEVQIVEEAT |
| Similar to | BC036755 | 4 | PSTLHSCHDIVLRRSYLGSWGFSIVGGYEENHTNQPFFIKTIVLG |
| Ligand of | | | TPAYYDGRLKCGDMIVAVNGLSTVGMSHSALVPMLKEQRNKV |
| Numb px2 | | | TLTVICWPGS |
| Similar to | 21595065 | 1 | SVTDGPKFEVKLKKNANGLGFSFVQMEKESCSHLKSDLVRIKRL |
| PTP | | | FPGQPAEENGAIAAGDIILAVNGRSTEGLIFQEVLH |
| Homolog | | | LLRGAPQEVTLLLCRPPPGA |
| SIP1 | 2047327 | 1 | QPEPLRPRLCRLVRGEQGYGFHLHGEKGRRGQFIRRVEPGSPAE |
| | | | AAALRAGDRLVEVNGVNVEGETHHQVVQRIKAVEGQTRLLVV |
| | | | DQETDEELRRRNSS |
| SIP1 | 2047327 | 2 | PLRELRPRLCHLRKGPQGYGFNLHSDKSRPGQYIRSVDPGSPAA |
| | | | RSGLRAQDRLIEVNGQNVEGLRHAEVVASIKAREDEARLLVVD |
| | | | PETDEHFKRNSS |
| SITAC-18 | 8886071 | 1 | PGVREIHLCKDERGKTGLRLRKVDQGLFVQLVQANTPASLVGL |
| | | | RFGDQLLQIDGRDCAGWSSHKAHQVVKKASGDKIVVVVRDRP |
| | | | FQRTVTM |
| SITAC-18 | 8886071 | 2 | PFQRTVTMHKDSMGHVGFVIKKGKIVSLVKGSSAARNGLLTNH |
| | | | YVCEVDGQNVIGLKDKKIMEILATAGNVVTLTIIPSVIYEHIVEFI |
| | | | V |
| SNPCIIA | 20809633 | 1 | SLERPRFCLLSKEEGKSFGFHLQQELGRAGHVVCRVDPGTSAQR |
| | | | QGLQEGDRILAVNNDVVEHEDYAVVVRRIRASSPRVLLTVLAR |
| | | | HAHDVARAQ |
| SNPCIIA | 20809633 | 3 | ISLPTKPRCLHLEKGPQGFGFLLREEKGLDGRPGQFLWEVDPGL |
| | | | PAKKAGMQAGDRLVAVAGESVEGLGHEETVSRIQGQGSCVSLT |
| | | | VVDPEADR |
| SNPCIIA | 20809633 | 4 | IPSVPLGSRQCFLYPGPGGSYGFRLSCVASGPRLFISQVTPGGSA |
| | | | ARAGLQVGDVILEVNGYPVGGQNDLERLQQLPEAEPPLCLKLA |
| | | | ARSLRGLE |
| Shank1 | 7025450 | 1 | LKEKTVLLQKKDSEGFGFVLRGAKAQTPIEEFTPTPAFPALQYLE |
| | | | SVDEGGVAWRAGLRMGDFLIEVNGQNVVKVGHRQVVNMIRQ |
| | | | GGNTLMVKVVMVTRHPDMDEAVQNSS |
| SYNTENIN | 2795862 | 1 | LEIKQGIREVILCKDQDGKIGLRLKSIDNGIFVQLVQANSPASLV |
| | | | GLRFGDQVLQINGENCAGWSSDKAHKVLKQAFGEKITMRIHRD |
| SYNTENIN | 2795862 | 2 | LRDRPFERTITMHKDSTGHVGFIFKNGKITSIVKDSSAARNGLLT |
| | | | EHNICEINGQNVIGLKDSQIADILSTSGTVVTITMPAFIFEHMNSS |
| Syntrophin 1 | 1145727 | 1 | QRRRVTVRKADAGGLGISIKGGRENKMPILISKIFKGLAADQTE |
| alpha | | | ALFVGDAILSVNGEDLSSATHDEAVQVLKKTGKEVVLEVKYMK |
| | | | DVSPYFK |
| Syntrophin | 476700 | 1 | PVRRVVKQEAGGLGISIKGGRENRMPILISKIFPGLAADQSRALR |
| beta 2 | | | LGDAILSVNGTDLRQATHDQAVQALKRAGKEVLLEVKFIRE |
| Syntrophin | 9507162 | 1 | EPFYSGERTVTIRRQTVGGFGLSIKGGAEHNIPVVVSKISKEQRA |
| gamma 1 | | | ELSGLLFIGDAILQINGINVRKCRHEEVVQVLRNAGEEVTLTVSF |
| | | | LKRAPAFLKLP |
| Syntrophin | 9507164 | 1 | SHQGRNRRTVTLRRQPVGGLGLSIKGGSEHNVPVVISKIFEDQA |
| gamma 2 | | | ADQTGMLFVGDAVLQVNGIHVENATHEEVVHLLRNAGDEVTIT |
| | | | VEYLREAPAFLK |
| TAX2-like | 3253116 | 1 | RGETKEVEVTKTEDALGLTITDNGAGYAFIKRIKEGSIINRIEAVC |
| Protein | | | VGDSIEAINDHSIVGCRHYEVAKMLRELPKSQPFTLRLVQPKRA |
| | | | F |
| TIAM 1 | 4507500 | 1 | HSIHIEKSDTAADTYGFSLSSVEEDGIRRLYVNSVKETGLASKKG |
| | | | LKAGDEILEINNRAADALNSSMLKDFLSQPSLGLLVRTYPELE |
| TIAM 2 | 6912703 | 1 | PLNVYDVQLTKTGSVCDFGFAVTAQVDERQHLSRIFISDVLPDG |
| | | | LAYGEGLRKGNEIMTLNGEAVSDLDLKQMEALFSEKSVGLTLIA |
| | | | RPPDTKATL |
| TIP1 | 2613001 | 1 | QRVEIHKLRQGENLILGFSIGGGIDQDPSQNPFSEDKTDKGIYVT |
| | | | RVSEGGPAEIAGLQIGDKIMQVNGWDMTMVTHDQARKRLTKR |
| | | | SEEVVRLLVTRQSLQK |
| TIP2 | 2613003 | 1 | RKEVEVFKSEDALGLTITDNGAGYAFIKRIKEGSVIDHIHLISVGD |
| | | | MIEAINGQSLLGCRHYEVARLLKELPRGRTFTLKLTEPRK |
| TIP33 | 2613007 | 1 | HSHPRVVELPKTDEGLGFNVMGGKEQNSPIYISRIIPGGVAERHG |
| | | | GLKRGDQLLSVNGVSVEGEHHEKAVELLKAAKDSVKLVVRYT |
| | | | PKVL |
| TIP43 | 2613011 | 1 | LSNQKRGVKVLKQELGGLGISIKGGKENKMPILISKIFKGLAADQ |
| | | | TQALYVGDAILSVNGADLRDATHDEAVQALKRAGKEVLLEVK |
| | | | YMREATPYVKNSS |
| Unknown | | 1 | QRSSIKTVELIKGNLQSVGLTLRLVQSTDGYAGHVIIETVAPNSP |
| PDZ gene | | | AAIADLQRGDRLIAIGGVKITSTLQVLKLIKQAGDRVLVYYERP |
| | | | VGQSNQGA |
| X-11 beta | 3005559 | 1 | IHFSNSENCKELQLEKHKGEILGVVVVESGWGSILPTVILANMM |
| | | | NGGPAARSGKLSIGDQIMSINGTSLVGLPLATCQGIIKGLKNQTQ |
| | | | VKLNIVSCPPVTTVLIKRNSS |
| X-11 beta | 3005559 | 2 | IPPVTTVLIKRPDLKYQLGFSVQNGIICSLMRGGIAERGGVRVGH |
| | | | RIIEINGQSVVATAHEKIVQALSNSVGEIHMKTMPAAMFRLLTG |
| | | | QENSS |
| ZO-1 | 292937 | 1 | IWEQHTVTLHRAPGFGFGIAISGGRDNPHFQSGETSIVISDVLKG |
| | | | GPAEGQLQENDRVAMVNGVSMDNVEHAFAVQQLRKSGKNAKI |
| | | | TIRRKKKVQIPNSS |
| ZO-1 | 292937 | 2 | ISSQPAKPTKVTLVKSRKNEEYGLRLASHIFVKEISQDSLAARDG |
| | | | NIQEGDVVLKINGTVTENMSLTDAKTLIERSKGKLKMVVQRDR |
| | | | ATLLNSS |
| ZO-1 | 292937 | 3 | IRMKLVKFRKGDSVGLRLAGGNDVGIFVAGVLEDSPAAKEGLE |
| | | | EGDQILRVNNVDFTNIIREEAVLFLLDLPKGEEVTILAQKKKDVF |
| | | | SN |
| ZO-2 | 12734763 | 1 | LIWEQYTVTLQKDSKRGFGIAVSGGRDNPHFENGETSIVISDVLP |
| | | | GGPADGLLQENDRVVMVNGTPMEDVLHSFAVQQLRKSGKVAA |
| | | | IVVKRPRKV |
| ZO-2 | 12734763 | 2 | RVLLMKSRANEEYGLRLGSQIFVKEMTRTGLATKDGNLHEGDII |
| | | | LKINGTVTENMSLTDARKLIEKSRGKLQLVVLRDS |
| ZO-2 | 12734763 | 3 | HAPNTKMVRFKKGDSVGLRLAGGNDVGIFVAGIQEGTSAEQEG |
| | | | LQEGDQILKVNTQDFRGLVREDAVLYLLEIPKGEMVTILAQSRA |
| | | | DVY |
| ZO-3 | 10092690 | 1 | IPGNSTIWEQHTATLSKDPRRGFGIAISGGRDRPGGSMVVSDVVP |
| | | | GGPAEGRLQTGDHIVMVNGVSMENATSAFAIQILKTCTKMANIT |
| | | | VKRPRRIHLPAEFIVTD |
| ZO-3 | 10092690 | 2 | QDVQMKPVKSVLVKRRDSEEFGVKLGSQIFIKHITDSGLAARHR |
| | | | GLQEGDLILQINGVSSQNLSLNDTRRLIEKSEGKLSLLVLRDRGQ |
| | | | FLVNIPNSS |
| ZO-3 | 10092690 | 3 | RGYSPDTRVVRFLKGKSIGLRLAGGNDVGIFVSGVQAGSPADG |
| | | | QGIQEGDQILQVNDVPFQNLTREEAVQFLLGLPPGEEMELVTQR |
| | | | KQDIFWKMVQSEFIVTD |

*: No GI number for this PDZ domain containing protein - it was computer cloned by J.S. using rat Shank3 seq against human genomic clone AC000036. In silico spliced together nt6400-6496, 6985-7109, 7211-7400 to create hypothetical human Shank3.

Vectors: All PDZ domain-containing genes were cloned into the vector pGEX-3X (Amersham Pharmacia #274803-01, Genemed Acc#U13852, GI#595717), containing a tac promoter, GST, Factor Xa, β-lactamase, and lac repressor.

The amino acid sequence of the pGEX-3X coding region including GST, Factor Xa, and the multiple cloning site is listed below. Note that linker sequences between the cloned inserts and GST-Factor Xa vary depending on the restriction endonuclease used for cloning. Amino acids in the translated region below that may change depending on the insertion used are indicated in small caps, and are included as changed in the construct sequence listed below.

aa 1-aa 232:

MSPILGYWKIKGLVQPTRLLLEYLEEKYEEHLYERDEGDKWRNKKFELGL
EFPNLPYYIDGDVKLTQSMAIIRYIADKHNMLGGCPKERAEISMLEGAVL
DIRYGVSRIAYSKDFETLKVDFLSKLPEMLKMFEDRLCHKTYLNGDHVTH
PDFMLYDALDVVLYMDPMCLDAFPKLVCFKKRIEAIPQIDKYLKSSKYIA
WPLQGWQATFGGGDHPPKSDLIEGRgipgnss

Constructs: The preparation of the construct for RIM2 (KIAA0751) is exemplified as flows. Constructs of the PDZ domains in Table 3 were prepared by similar methods. Primers used to generate RIM2 DNA fragments by PCR are listed in Table 4. PCR primer combinations and restriction sites for insert and vector are listed below, along with amino acid translation for insert and restriction sites. Non-native amino acid sequences are shown in lower case.

TABLE 4
Primers used in cloning of RIM2 PDZ domain 1.

| ID# (Primer | Primer | | Seq |
|---|---|---|---|
| Name) | Sequence | Description | ID |
| 1968 | AAAGATCTCCCTTA | Forward (5′ to 3′) primer corresponding to | 273 |
| (688KIFlo) | ACGAGGAGCATAG | RIM2, domain 1. Generates a BglII site | |
| | | upstream (5′) of the PDZ boundary. Used | |
| | | for cloning into pGEX-3X. | |
| 1093 | GAACAATTGCAATA | Reverse (3′ to 5′) primer corresponding to | 274 |
| (319KIR) | GGCCTTGAAACTAC | RIM2, domain 1. Generates a MfeI site | |
| | | downstream (3′) of the PDZ boundary. Used | |
| | | for cloning into pGEX-3X. | |

RIM2, PDZ domain 1: GI#: 12734165; Construct: RIM2, PDZ domain 1-pGEX-3×; primers: 1968 & 1093; Vector Cloning Sites (5′/3′): Bam H1/EcoR1; Insert Cloning Sites(5′/3′): BglII/MfeI

aa 1-aa 126

TLNEEHSHSDKHPVTWQPSKDGDRLIGRILLNKRLKDGSVPRDSGAMLGL
KVVGGKMTESGRLCAFITKVKKGSLADTVGHLRPGDEVLEWNGRLLQGAT
FEEVYNIILESKPEPQVELVVSRPIG

GST Fusion Protein Production and Purification: The constructs using pGEX-3X expression vector were used to make fusion proteins according to the protocol outlined in the GST Fusion System, Second Edition, Revision 2, Pharmacia Biotech. Method II and optimized for a IL LGPP.

Purified DNA was transformed into E. coli and allowed to grow to an OD₆₀₀ of 0.4-0.8 (600λ). Protein expression was induced for 1-2 hours by addition of IPTG to cell culture. Cells were harvested and lysed. Lysate was collected and GS4B beads (Pharmacia Cat#17-0756-01) were added to bind GST fusion proteins. Beads were isolated and GST fusion proteins were eluted with GEB II. Purified proteins were stored in GEB II at −80° C.

Purified proteins were used for ELISA-based assays and antibody production.

### Example 7

### Identification of PDZ Domains Bound by the C-Terminus of MUC1

Summary: To determine the human PDZ domains bound by the C-terminus of MUC1, peptides corresponding to the PL (20 amino acids of the C-terminus (SEQ ID NO: 96) or 9 amino acids of the C-terminus coupled to 11 amino acids of the TAT transporter (SEQ ID NO: 102) were synthesized and purified to >95% by HPLC. These peptides were assessed for binding to individual GST-PDZ domain fusion proteins using the modified ELISA describe below. Interactions giving higher absorbance values in the assay were titrated to determine relative EC50 values.

Reagents and Supplies:

### Nunc MaxiSorp 96 well Immuno-plate, Nunc;

### PBS pH 7.4 (phosphate buffered saline, 8 g NaCl, 0.29 g KCl, 1.44 g Na₂HPO₄, 0.24 g KH₂PO₄, add H₂O to 1 L and pH 7.4; 0.2μ filter) Assay Buffer: 2% BSA in PBS (20 g of bovine serum albumin per liter PBS, fraction V, ICN Biomedicals, cat#IC15142983

### Goat anti-GST polyclonal Ab, stock 5 mg/ml, stored at 4° C., Amersham Pharmacia cat#27-4577-01;

### Dilute 1:1000 in PBS, final concentration 5 μg/ml;

### HRP-Streptavidin, 2.5 mg/2 ml stock stored at 4° C., Zymed cat#43-4323, dilute 1:2000 into Assay buffer, final [0.5 μg/ml]

### Wash Buffer, PBS;

### Biotinylated peptides (HPLC purified, stock solution store in −20° C. freezer #7)

### GST-PRISM proteins (stock stored at −80° C., after first thaw store in −10° C. freezer #7)

### TMB (3,3′,5,5′, teramethylbensidine), tablets, Sigma cat.#T5525;

### Per plate, dissolve 1 tablet in 1 mL DMSO, add 9 mL Citrate/Phosphate buffer pH 5.4 and 2 μL

### H₂O₂;

### 0.18M H₂SO₄, Sigma cat.#S1526;

12-w multichannel pipettor & tips;
50 ml reagent reservoirs, Costar#4870;
50, 15 ml polypropylene conical tubes;

### Costar Transtar 96 Costar#7605;

### Transtar 96 Cartridge Costar#7610;

### Cluster tubes;

### Molecular Devices microplate reader (450 and 650 nm filters);

### SoftMax Pro software;

### When using reagents stored at or 4° C. or −20° C., remove and keep on ice Protocol:

### Coat plate with 100 μl of 5 μg/ml anti-GST, O/N at 4° C.;

### Dump contents of plate & out tap dry on paper towels;

### Block with 200 μl Assay Buffer for 2 hrs at room temperature;

### Prepare proteins in Assay Buffer;

### Wash 3× with cold PBS*;

### Add proteins at 50 μl per well, incubate 1 to 2 hrs at 4° C.;

### Prepare peptides in Assay Buffer;

### Wash 3× with cold PBS*;

### Add peptides at 50 μl per well on ice (write time on plate);

### Incubate on ice after last peptide has been added for exactly 10 minutes;

### Place at room temp for exactly 20 minutes;

### Prepare HRP-Streptavidin within 10 minutes of time of use;

### Promptly wash 3× with cold PBS;

### Add 100 μl per well of HRP-Streptavidin (write time on plate);

### Incubate at 4° C. for exactly 20 minutes;

### Turn on plate reader and prepare files;

### Promptly wash 5× with PBS at room temperature;

### Add 100 μl/well TMB substrate (write time on plate);

### Incubate in dark at room temp for a maximum of 30 minutes;

### Read plate at 25 minutes (650 nm);

### Stop reaction with 100 μl of 0.18M H₂SO₄, 30 min. after adding TMB;

### Take last reading at 450 nm soon after stopping reaction;

*do not let plates dry out

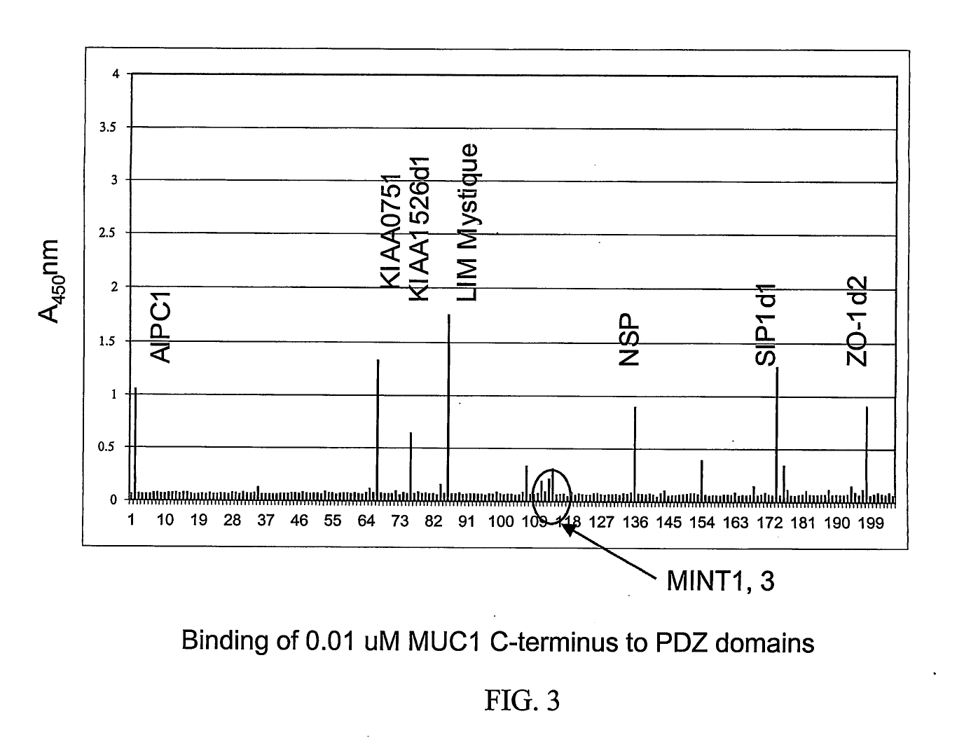

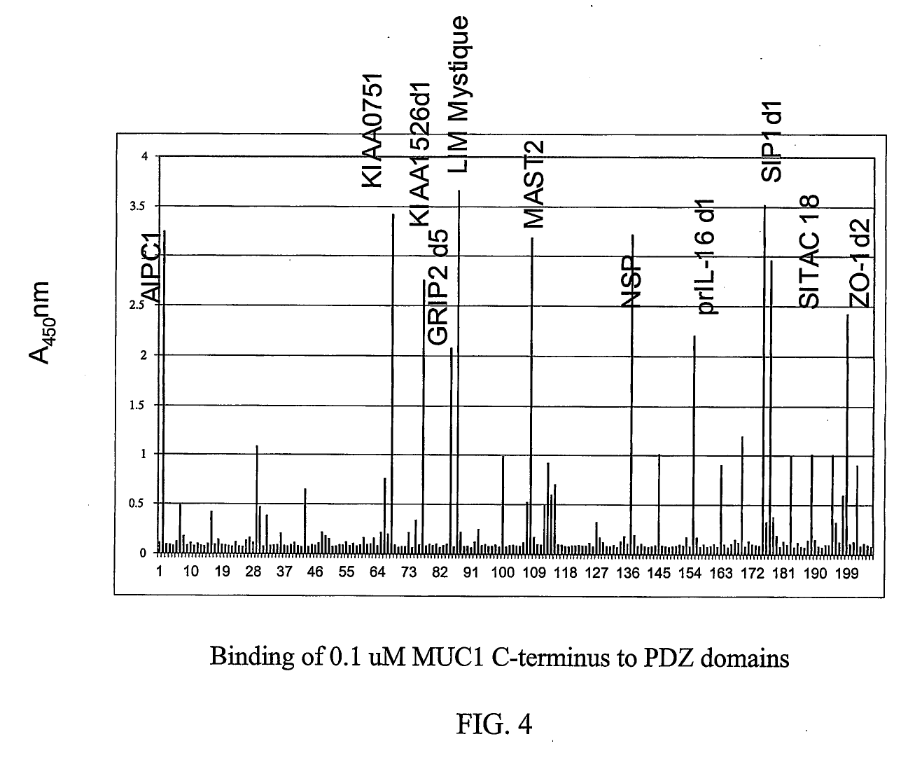

Profile Results Peptides corresponding to the C-terminus were able to bind a number of PDZ domains in a concentration dependent manner. FIG. 3 shows the results of MUC1 binding to individual PDZ domains at a MUC1 peptide concentration of 0.01 μm, and seven interactions are observed to give higher absorbance readings in the assay. When the concentration of MUC1 peptide is increased to 0.1 μm, more interactions are observed (FIG. 4). Identities of the interacting PDZ domains are listed directly above or next to the bar representing the absorbance in the assay.

Titrations to determine relative EC₅₀ values: Peptide corresponding to MUC1 was then titrated against a constant amount of the PDZ domain-containing recombinant proteins identified in the first part of this example. From these, relative EC₅₀ values are listed in Table 4 indicating the concentration of MUC1 peptide for 50 percent binding to the indicated PDZ domain

TABLE 4

| PDZ | EC50 μM |
|---|---|
| Lim Mystique | 0.010 |
| SIP1 d1 | 0.011 |
| AIPC1 | 0.014 |
| KIAA0751 | 0.016 |
| ZO-1 d2 | 0.019 |
| SITAC 18 | 0.026 |
| NSP | 0.027 |
| MAST d2 | 0.039 |
| Pril-16 d1 | 0.041 |
| KIAA1526 d1 | 0.051 |
| GRIP2 d5 | 0.060 |

The C-terminus of MUC1 clearly functions as a PDZ ligand and several PDZ domains can bind to the MUC1 C-terminus, and modulation of these interactions provide a point of therapeutic intervention.

### Example 8

### Expression of PDZ Domains in Human Cancer Cells

Expression of PDZ domains in breast cancer cell lines was examined using quantitative PCR to confirm that PDZ domains shown to interact with the C-terminus of MUC1 are present in cancer cell lines.

Methods: cDNA was prepared from 4 cell lines using standard methods: human breast cancer MCF-7 cells; human breast cancer ZR-75 cells; human colon cancer HCT116 cells transfected with MUC1; human colon cancer HCT116 cells transfected with vector as a control. HCT116 cells do not express MUC1 endogenously. MUC1 transfection of HCT116 cells is described in U.S. Patent Application Publication 2004/0018181 A1, incorporated herein by reference. Amplicon primer pairs were designed using software provided with our ABI7000 Real Time PCR machine. Reactions performed in duplicate, and were repeated independently.

Cells were grown under respective growth conditions to 80% confluency. Total RNA was isolated using TRIZOL and standard protocols. cDNA was generated by using Superscript Reverse Transcriptase and random primers (Invitrogen). Real time PCR was performed on the cDNAs utilizing the SYBR GREEN method (ABI) and quantified in an ABI PRISM 7000 Sequence detection system. Relative expression is based on copy numbers for an EGFR Plasmid/Amplicon primer pair which was used for a standard curve (from 1 million to 320 copies) which was included in each individual plate. Values >200 were considered significant over background. Also included in each plate was a beta-Actin control for each of the four cell types. Minus RT controls were also included and each individual plate contained a non-template control using beta-Actin primers. Amplicon primers were designed using the ABI Primer Design software and corresponded to sequences within the respective hit-PDZ except for MINT-3 where a sequence outside the PDZ domain was used. Reactions were done in duplicates and for all genes which showed no expression, a second independent primer pair within the PDZ sequence (except for MINT-3) was designed and checked against the cDNAs. In addition, each negative primer pair was checked against the respective PDZ Plasmid to confirm whether the primer pair is functional. For all primer pairs except for the GRIP-2 primers functionality was confirmed with the Plasmids. Table 5 shows the primers used to determine PDZ gene expression in ZR-75, MCF7 and HCT116+/−MUC1 transgene expression cell lines.

TABLE 5
Oligonucleotide primers used for RT-PCR

| AVC | | | |
|---|---|---|---|
| No | Oligo Name | Sequence | Description |
| 3303 | Zo-3 dom3 FA | gcatccaggagggagatcag | forward amplicon primer |
| 3302 | Zo-3 dom3 RA | aggttctggaatggcacgtc | reverse amplicon primer |
| 3301 | Zo-3 dom3 FB | gggcatccaggagggagat | forward amplicon primer |
| 3300 | Zo-3 dom3 RB | caggttctggaatggcacg | reverse amplicon primer |
| 3299 | Zo-3 dom1 FA | caggcgaccacatcgtcat | forward amplicon primer |
| 3298 | Zo-3 dom1 RA | gaggtggcattctccatgga | reverse amplicon primer |
| 3297 | Zo-3 dom1 FB | tccatggagaatgccacctc | forward amplicon primer |
| 3296 | Zo-3 dom1 RB | ccatcttggtgcaggtcttga | reverse amplicon primer |
| 3295 | Zo-2 dom1 FA | agtggtcatggtcaatggca | forward amplicon primer |
| 3294 | Zo-2 dom1 RA | gcaaacgaatgaagcacatcc | reverse amplicon primer |
| 3293 | Zo-2 dom1 FB | ctgatgggctgctccaaga | forward amplicon primer |
| 3292 | Zo-2 dom1 RB | gggtgccattgaccatgac | reverse amplicon primer |
| 3291 | Zo-2 dom2 FA | agtatggtctccggcttggg | forward amplicon primer |
| 3290 | Zo-2 dom2 RA | ttcgggtcatttcctttacga | reverse amplicon primer |
| 3289 | Zo-2 dom2 FB | gatgaaaagcagagcgaacga | forward amplicon primer |
| 3288 | Zo-2 dom 2 RB | cgaagatctgactcccaagcc | reverse amplicon primer |
| 3252 | KIA0340 DOM 1 2ND R | caccaagtcgtcctaagtcagtcat | reverse amplicon primer |
| 3251 | KIA0340 DOM 1 2ND F | tgggtctgaaagttgttggagg | forward amplicon primer |
| 3250 | GRIP2 DOM 5 2ND R | cagttgtccaggcggatattg | reverse amplicon primer |
| 3249 | GRIP2 DOM 5 2ND F | ggagccaggcgacaagc | forward amplicon primer |
| 3248 | LIM MYST DOM 1 2ND | cgttgatggccacgattatgt | reverse amplicon primer |
| | R | | |
| 3247 | LIM MYST DOM 1 2ND | aaagccaaggacgctgacct | forward amplicon primer |
| | F | | |
| 3246 | KIA0316 DOM 1 2ND R | aggagtatcgattctttgcagctt | reverse amplicon primer |
| 3245 | KIA0316 DOM 1 2ND F | cagagagcgggtcatcgatc | forward amplicon primer |
| 3244 | MAGI2 DOM5 2ND R | tcctaccctcatcctcccatt | reverse amplicon primer |
| 3243 | MAGI2 DOM5 2ND F | agactggcagaagatggacca | forward amplicon primer |
| 3242 | MAST1 DOM 1 2ND R | tccgtgtcacccatgtagacac | reverse amplicon primer |
| 3241 | MAST1 DOM 1 2ND F | gaagtatggcttcacactgcgt | forward amplicon primer |
| 3240 | MINT3 COMPL 2ND R | catgcctggactccaggct | reverse amplicon primer |
| 3239 | MINT3 COMPL 2ND F | cgatttgggaactgcctgaa | forward amplicon primer |
| 3238 | MUPP1 DOM 3 2ND R | caatgtagccagcaatggtaattc | reverse amplicon primer |
| 3237 | MUPP1 DOM 3 2ND F | gaactcactaaaaatgtccaaggattag | forward amplicon primer |
| 3236 | NOVEL PDZ DOM 1 | ccatggtggtgctctccag | reverse amplicon primer |
| | 2ND R | | |
| 3235 | NOVEL PDZ DOM 1 | gggacaagatcacggaggtg | forward amplicon primer |
| | 2ND F | | |
| 3234 | NSP DOM 1 2ND R | cgctcctgagatcacgtctg | reverse amplicon primer |
| 3233 | NSP DOM 1 2ND F | aaagagctgaaggaccggc | forward amplicon primer |
| 3232 | HER1 2ND R | tggccatcacgtaggcttc | reverse amplicon primer |
| 3231 | HER1 2ND F | agcaacatctccgaaagcca | forward amplicon primer |
| 3230 | SYNTROPHINY DOM 1 | tcagctgcttggtcttcgaat | reverse amplicon primer |
| | R | | |
| 3229 | SYNTROPHINY DOM 1 | gcacaacgtccctgtcgtc | forward amplicon primer |
| | F | | |
| 3228 | PRIL16 DOM 1 R | cgtggtccccttgagagactt | reverse amplicon primer |
| 3227 | PRIL16 DOM 1 F | aagggcaatgaggttctttcc | forward amplicon primer |
| 3226 | KIA 1719 DOM 5 R | gcagttgtccaggcggata | reverse amplicon primer |
| 3225 | KIA 1719 DOM 5 F | gagccaggcgacaagctact | forward amplicon primer |
| 3224 | KIA1526 DOM 1 R | cccgcagtccttccttctc | reverse amplicon primer |
| 3223 | KIA1526 DOM 1 F | acgtgtctctggtggaaccag | forward amplicon primer |
| 3222 | FGFR3 IIIC B NEW R | gcacgtccagcgtgtacgt | reverse amplicon primer |
| 3221 | FGFR3 IIIC B NEW F | tgcgtcgtggagaacaagttt | forward amplicon primer |
| 3220 | FGFR3 IIIC A NEW R | acgtccagcgtgtacgtctg | reverse amplicon primer |
| 3219 | FGFR3 IIIC A NEW F | cgtcgtggagaacaagtttgg | forward amplicon primer |
| 3218 | HER2 B NEW R | ccacttgatgggcaccttg | reverse amplicon primer |
| 3217 | HER2 B NEW F | ctgctggacattgacgagaca | forward amplicon primer |
| 3216 | HER2 A NEW R | ctgtgtacgagccgcacatc | reverse amplicon primer |
| 3215 | HER2 A NEW F | ctggtgtatgcagattgccaa | forward amplicon primer |
| 3214 | VARTUL COMPLETE R | cagatcgttgcctcccagat | reverse amplicon primer |
| 3213 | VARTUL COMPLETE F | cgtccctgtcatttctggtca | forward amplicon primer |
| 3212 | SITAC18 DOM 1 R | tgccttcttcaccacctgatg | reverse amplicon primer |
| 3211 | SITAC18 DOM 1 F | gactgtgctgggtggagctc | forward amplicon primer |
| 3210 | DLG 1 DOM 2 R | cccaggaatatgctgatttcca | reverse amplicon primer |
| 3209 | DLG 1 DOM 2 F | ggtcttgggtttagcattgctg | forward amplicon primer |
| 3208 | DLG 1 DOM1 R | tctccaatgtgtgggttgtcc | reverse amplicon primer |
| 3207 | DLG 1 DOM 1 F | tcagggcttggtttcagcat | forward amplicon primer |
| 3206 | Ubiquitin R | caattgggaatgcaacaactttat | reverse amplicon primer |
| | Chamorro | | |
| 3205 | Ubiquitin F | cacttggtcctgcgcttga | forward amplicon primer |
| | Chamorro | | |
| 3204 | Ubiquitin F | aatcatttgggtcaatatgtaattttca | forward amplicon primer |
| 3203 | Ubiquitin R | gcggacaatttactagtctaacactga | reverse amplicon primer |
| 3202 | 18S RNA R | gggtcgggagtgggtaattt | reverse amplicon primer |
| 3201 | 18S RNA F | ctaccacatccaaggaaggca | forward amplicon primer |
| 3200 | PTPL1 dom4 R | cttttggctggatcctgtatgac | reverse amplicon primer |
| 3199 | PTPL1 dom4 F | tcagagaattggttgttatgttcatg | forward amplicon primer |
| 3198 | Mupp1 dom 6 R | tccggccatctcgactaatg | reverse amplicon primer |
| 3197 | Mupp1 dom 6 F | gggatgatcgttcgaagcat | forward amplicon primer |
| 3196 | Mast 3 com 1 R | agacgtcgctatcacccatgt | reverse amplicon primer |
| 3195 | Mast 3 dom 1 F | tggcaagaagtacggcttca | forward amplicon primer |
| 3194 | Kia340 dom 1 R | aacaactttcagacccagcaatg | reverse amplicon primer |
| 3193 | Kia340 dom 1 F | agaacaaccatgcccaaagact | forward amplicon primer |
| 3192 | INADL dom 3 R | cctgccctgcatttcgtaa | reverse amplicon primer |
| 3191 | INADL dom 3 F | cagggttttgccaaccatg | forward amplicon primer |
| 3190 | PAR 3 dom 3 R | gcccaacagggattctccat | reverse amplicon primer |
| 3189 | PAR3 dom 3 F | ggcttcgggtgaatgatcaa | forward amplicon primer |
| 3188 | Pick 1 dom 1 R | cttcgccacctccaccttag | reverse amplicon primer |
| 3187 | Pick 1 dom 1 F | ggtgtcaatggcaggtcaatc | forward amplicon primer |
| 3186 | RGS3 dom 1 R | gaatccacggcctggactc | reverse amplicon primer |
| 3185 | RGS3 dom 1 F | tggcttcaccatctgctgc | forward amplicon primer |
| 3184 | Sip 1 dom 1 R | cagccttgatcctttgcacc | reverse amplicon primer |
| 3183 | Sip 1 dom 1 F | gtcaacgtggagggcgag | forward amplicon primer |
| 3182 | SIP1 dom 2 R | gccgggacttgtcactatgc | reverse amplicon primer |
| 3181 | SIP 1 dom 2 F | gaaagggacctcagggctatg | forward amplicon primer |
| 3180 | Tip 1 R | ccaatgctgaaacccaggat | reverse amplicon primer |
| 3179 | Tip 1 F | aattcacaagctgcgtcaagg | forward amplicon primer |
| 3178 | AIPC dom 1 F | gggccttggctttagtattgc | forward amplicon primer |
| 3177 | Mint 3 500 bp R | cagctggcatcgtcttgatatg | reverse amplicon primer |
| 3176 | Mint 3 500 bp F | agctgctcaccgaggcctat | forward amplicon primer |
| 3175 | Mint 1 dom2 R | cgcatgaggctgcagataatt | reverse amplicon primer |
| 3174 | Mint 1 dom2 F | ctaccagctcggtttcagcg | forward amplicon primer |
| 3173 | Mint 1 dom1 R | tctggcaggtggacagagg | reverse amplicon primer |
| 3172 | Mint 1 dom1 F | cggtgaccagatcatgtccat | forward amplicon primer |
| 3171 | PTN3 R | acgatttgatccccttcgttc | reverse amplicon primer |
| 3170 | PTN3 F | agtcacctgcggacacctg | forward amplicon primer |
| 3169 | HTRA2 R | gggaaagcttggttctcgaag | reverse amplicon primer |
| 3168 | HTRA2 F | ctgagtcccagcatccttgc | forward amplicon primer |
| 3167 | AIPC dom 1 R | ccccatctgtccacgaatg | reverse amplicon primer |
| 3166 | Mast 2 dom 1 F | acttcttgccagcccttgg | forward amplicon primer |
| 3165 | Mupp1 dom 3 R | ttggtctccaatttggattcttc | reverse amplicon primer |
| 3164 | Mupp1 dom 3 F | acaaaaagcagtgccgttga | forward amplicon primer |
| 3163 | Novel PDZ dom 1 R | cagcacctttacggcgctac | reverse amplicon primer |
| 3162 | Novel PDZ dom 1 F | aatgggctgagcctggaga | forward amplicon primer |
| 3161 | MAGI 2 dom 5 F | tgtggacatggagaaaggagc | forward amplicon primer |
| 3160 | Mast 1 dom 1 R | tgccagacaatgtggtggac | reverse amplicon primer |
| 3159 | Mast 1 dom 1 F | tgtctacatgggtgacacgga | forward amplicon primer |
| 3158 | Mast 2 dom 1 R | gctcggtggatgatgatgg | reverse amplicon primer |
| 3157 | NSP dom 1 R | tcctgagatcacgtctgggaa | reverse amplicon primer |
| 3156 | NSP dom 1 F | aagccaaagagctgaaggacc | forward amplicon primer |
| 3155 | Elfin 1 dom 1 R | ccttgcttccaggagtgacc | reverse amplicon primer |
| 3154 | Elfin 1 dom 1 F | aaaggacttcgagcagcctct | forward amplicon primer |
| 3153 | EBP50 dom 2 R | tccactgaccggatgaactg | reverse amplicon primer |
| 3152 | EBP50 dom 2 F | caacctgcacagcgacaagt | forward amplicon primer |
| 3151 | ZO 1 dom 2 R | gcttgccaatcgaagaccat | reverse amplicon primer |
| 3150 | ZO 1 dom 2 F | acactggtgaaatcccggaa | forward amplicon primer |
| 3149 | EBP50 dom 1 R | tgtactggcccaacttgcc | reverse amplicon primer |
| 3148 | EBP50 dom 1 F | agaagggtccgaacggctac | forward amplicon primer |
| 3147 | APXL dom 1 R | cgcttcctgtctaaaccctga | reverse amplicon primer |
| 3146 | APXL1 dom 1 F | tgagatcgtcggcatcaatg | forward amplicon primer |
| 3145 | Grip 2 dom 5 R | gcagttgtccaggcggata | reverse amplicon primer |
| 3144 | Grip 2 dom 5 F | gagccaggcgacaagctact | forward amplicon primer |
| 3143 | KIA0382 dom 1 R | atggctgctccatcttctttg | reverse amplicon primer |
| 3142 | KIA0382 dom 1 F | cggtcagtggagacaatcca | forward amplicon primer |
| 3141 | Erbin dom 1 R | acaccacctgatatgctaaatcca | reverse amplicon primer |
| 3140 | Erbin dom 1 F | agtgagggttgaaaaggatcca | forward amplicon primer |
| 3139 | KIA0316 dom 1 R | tgaccagatcgatgacccg | reverse amplicon primer |
| 3138 | KIA0316 dom1 F | aatgatgaaccggtcagcg | forward amplicon primer |
| 3137 | KIA0751 (RIM2) | aaagccgacctgattcagtca | reverse amplicon primer |
| | dom1 R | | |
| 3136 | KIA0751 (RIM2) | caatgcttggcttgaaggttg | forward amplicon primer |
| | dom 1 F | | |
| 3135 | Lim Mystique dom 1R | ccgttgatggccacgattat | reverse amplicon primer |
| 3134 | Lim Mystique dom 1F | agccaaggacgctgacctc | forward amplicon primer |
| 3133 | Lim Protein dom1 R | ccttgccgccatcttttaga | reverse amplicon primer |
| 3132 | Lim Protein dom1 F | cggtaaggatttcaacatgcc | forward amplicon primer |
| 3131 | MAGI 2 dom 5 R | cctccacgaatgctgaatcc | reverse amplicon primer |
| 3116 | AIPC As (reverse) | gctgatccatttgggaagatg | Amplicon primer for |
| | | | real-time PCR |
| 3115 | AIPC S (forward) | gcattcgtggacagatggg | Amplicon primer for |
| | | | real-time PCR |
| 3114 | HER 1 As (reverse) | cagggattccgtcatatggct | Amplicon primer for |
| | | | real-time PCR |
| 3113 | HER 1 S (forward) | ccgtttgggagttgatgacc | Amplicon primer for |
| | | | real-time PCR |
| 3112 | HER 2 As (reverse) | ccacttgatgggcaccttg | Amplicon primer for |
| | | | real-time PCR |
| 3111 | HER 2 S (forward) | tgctggacattgacgagacag | Amplicon primer for |
| | | | real-time PCR |
| 3110 | FGFR3C AS (reverse) | cacgtccagcgtgtacgtct | Amplicon primer for |
| | | | real-time PCR |
| 3109 | FGFR3C S (forward) | ctgcgtcgtggagaacaagtt | Amplicon primer for |
| | | | real-time PCR |
| 3108 | b-Catenin AS | gctgggtatcctgatgtgca | Amplicon primer for |
| | (reverse) | | real-time PCR |
| 3107 | b-Catenin S | gggtgccattccacgactag | Amplicon primer for |
| | (Forward) | | real-time PCR |
| 3106 | MUC-1 AS (reverse) | tgtccagctgcccgtagttc | Amplicon primer for |
| | | | real-time PCR |
| 3105 | MUC-1 S (forward) | ttgccttggctgtctgtcag | Amplicon primer for |
| | | | real-time PCR |
| 3414 | RIM2 P7R | tgtggttcaggtttggattctagaa | |
| 3413 | RIM2 P7F | cacatttgaggaagtgtacaacatcat | |
| 3412 | RIM2 P6R | tggctccttgcagtagtcttcc | |
| 3411 | RIM2 P6F | gaccaggtgatgaagtattagaatgg | |
| 3410 | RIM2 P5R | ccaccaaagtacatcatttcctttt | |
| 3409 | RIM2 P5F | gtcggactctaacaccaggtctg | |
| 3408 | RIM2 P4R | tggccaccaaagtacatcatttc | |
| 3407 | RIM2 P4F | ctctaacaccaggtctgagagacaaa | |
| 3406 | RIM2 P3R | ttggttccatttgggttcca | |
| 3405 | RIM2 P3F | ttccagacagaagtgataaaaacaagag | |
| 3404 | RIM2 P2R | tgcattgttcagtgtttgtcca | |
| 3403 | RIM2 P2F | ccaccaaatatcttacaaaatgagctt | |
| 3402 | RIM2 P1R | tccagatcagcatttgccaa | |
| 3393 | RIM2 P1F | acggcatgagagaaggcatag | |

Results: Table 6 shows the RNA expression in four cell lines as described utilizing the primers listed in Table 5. The results indicate that several of the target PDZ mRNAs are expressed in the selected cancer cell lines and are potential targets for therapeutic intervention. In the case of RIM2, alternatively spliced genes were observed; however, the primer sets indicate that the PDZ domain is expressed in these cell lines.

In Table 6, “+” is indicative of expression, and “−” is indicative of low or no expression. * —denotes that different primer pairs were used, corresponding to the pairs listed at the bottom of Table 5. For example, RIM2 P1 was evaluating RNA expression using RIM2 P1F (forward) and RIM2 PIR (reverse) primers.

TABLE 6
RNA expression in cell lines

| | | HCT116 | | |
|---|---|---|---|---|
| | HCT116 | MUC1 | MCF-7 | ZR-75 |
| BETA-CATENIN | + | + | + | + |
| FGFR3IIIC | + | + | + | + |
| HER1 | + | + | + | + |
| HER2 | + | + | + | + |
| MUC1 | − | + | + | + |
| AIPC d1 | − | − | − | + |
| APXL d1 | + | + | + | + |
| DLG1 d1 | + | + | + | + |
| DLG1 d2 | + | + | + | + |
| EBP50 d1 | + | + | + | + |
| EBP50 d2 | + | + | + | + |
| ELFIN d1 | + | + | + | + |
| ERBIN d1 | + | + | + | + |
| GRIP2 d5 | − | − | − | − |
| HTRA2 d1 | + | + | + | + |
| INADL d3 | + | + | + | + |
| KIA0316 d1 | − | − | − | − |
| KIA0340 d1 | − | − | − | − |
| KIA0382 d1 | + | + | + | + |
| KIA0751 d1* | − | − | − | + |
| KIA1526 d1 | − | − | − | − |
| LIM MYSTIQUE d1 | + | + | + | + |
| LIM PROTEIN d1 | + | + | + | + |
| MAGI2 d5 | − | − | + | + |
| MAGI3 d5 | + | + | + | + |
| MAST1 d1 | − | − | − | − |
| MAST2 d1 | + | + | − | + |
| MAST3 d1 | + | + | + | + |
| MINT1 d1 | − | − | − | + |
| MINT1 d2 | − | − | − | − |
| MINT3 full-length | − | − | − | − |
| MUPP1 d3 | − | − | + | + |
| MUPP1 d6 | − | − | + | + |
| NOVEL PDZ d1 | − | − | − | − |
| NSP d1 | − | − | − | − |
| PAR3 d3 | + | + | + | + |
| PICK1 d1 | + | + | + | + |
| prIL-16 d1 | − | − | − | + |
| PTN3 d1 | + | + | + | + |
| PTPL1 d4 | + | + | + | + |
| RGS3 d1 | + | + | + | + |
| SIP1 d1 | + | + | + | + |
| SIP1 d2 | + | + | + | + |
| SITAC18 d1 | − | − | − | − |
| SYNTROPHINy d1 | − | − | − | − |
| TIP1 d1 | + | + | + | + |
| VARTUL d4 | + | + | + | + |
| ZO-1 d2 | + | + | + | + |
| RIM2 P1* | + | + | + | + |
| RIM2 P2* | + | + | + | + |
| RIM2 P3* | +/− | +/− | + | + |
| RIM2 P4* | + | + | + | + |
| RIM2 P5* | + | + | + | + |
| RIM2 P6* | + | + | + | + |
| RIM2 P7* | + | + | + | + |

### Example 9

### Knockdown of MUC1 Binding PDZ Proteins in Cancer Cells

The effects of knocking-down the PDZ domain proteins ZO-1, SIP1, LIM Mystique and KIAAO751 by siRNAs on anti-apoptotic function of MUC1 were examined in human non-small cell lung cancer A549 cells that endogenously express MUC1 and transfected human colon cancer HCT116 cells that exogenously express MUC1.

Cell culture and transfection: Human colon cancer HCT116 cells and human non-small lung cancer A549 were grown in Dulbecco's modified Eagle's medium (DMEM) and RPMI1640 medium, respectively, in a humidified 5% CO₂ atmosphere at 37° C. Media were supplemented with 10% fetal bovine serum (FBS), 100 units/ml of penicillin and 100 μg/ml of streptomycin. HCT116 cells were transfected with pIRES-puro2 or pIRES-puro2-MUC1 as described (Li et al., 2001(a)) and stable transfectants were selected in the presence of 0.4 μg/ml puromycin (Caliochem-Novabiochem).

Generation of siRNA for transfection: siRNAs were synthesized to knock-down expression of LIM-M (GI: 28866956), KIAA0751 (GI: 3882222), ZO-1 (GI: 28416399) and SIP1 (GI: 2047327) (Dharmacon, Inc.). The targeted sequences for these genes were as follows:

LIM Mystique:
5′-AAGCTGGTGAGACAACCTCTG-3′
KIAA0751:
5′-AACACCAGGTCTGAGAGACAA-3′
ZO-1:
5′-AAGTTGGCAACCAGATGTGGA-3′
SIP1:
5′-AAGCTGGCAAGAAGGATGTCA-3′

A nonspecific scrambled control siRNA (SCRsiRNA) was also synthesized (targeted sequence: 5′-AAGCGCGCTTTGTAGGATTCG-3′) (Dharmacon, Inc.). Cells were plated, grown in antibiotic-free medium overnight, and then transiently transfected with siRNAs (0.2-20 nM) using Oligofectamine reagent (Invitrogen Life Technology, Inc.) and Opti-MEM 1 reduced serum medium (Invitrogen Life Technology, Inc.) according to the manufacturer's instructions.

Apoptosis assay: At 48 hr after siRNA transfection, cells were treated with 0, 10 or 100 μM cisplatin (CDDP, Sigma) for 24 hr to induce apoptosis. Apoptotic cells were quantified by analysis of sub-G1 DNA content. Cells were harvested, washed, with PBS, fixed with 75% ethanol, and incubated in PBS containing 200 μg/ml RNase A (Qiagen) for 15 min at 37° C. Cells were then stained with 50 ug/ml propidium iodide (Boeringer Manheim) for 30 min at room temperature in the dark. DNA content was analyzed by flow cytometry (EPICS XL-MCL, Coulter Corp.).

Immunoblotting: Cells were incubated for the indicated times, harvested, washed with ice-cold PBS, and lysed in lysis buffer [150 mM NaCl, 50 mM Tris (pH 7.6), 5 mM EDTA, 0.5% NP-40, and protease inhibitor cocktail (Complete, Roche Diagnostics Corp)]. Whole cell lysates were subjected to SDS-PAGE, transferred to nitrocellurose membrane, and immunoblotted with antibodies against KIAA0751 (Rim2, Santa Cruz Biotechnology), SIP1 (NHERF2, Alpha Diagnostic International), ZO-1 (Zymed Laboratories) or β-actin (Clone AC-15; Sigma). The blots were developed by using the ECL kit (Amersham Pharmacia Biotech).

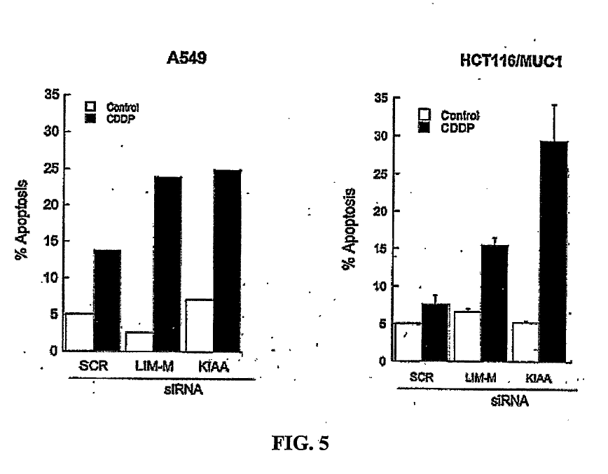

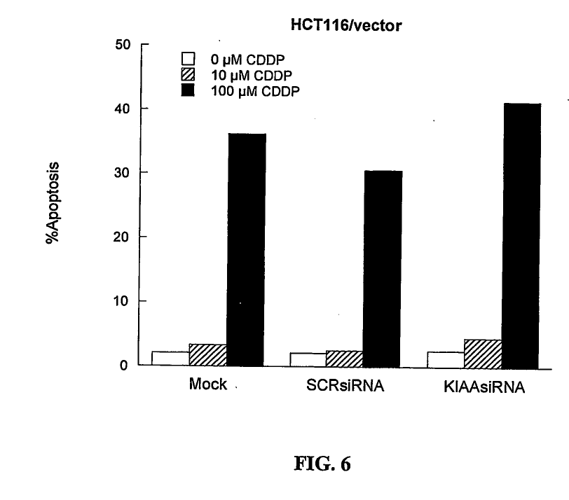

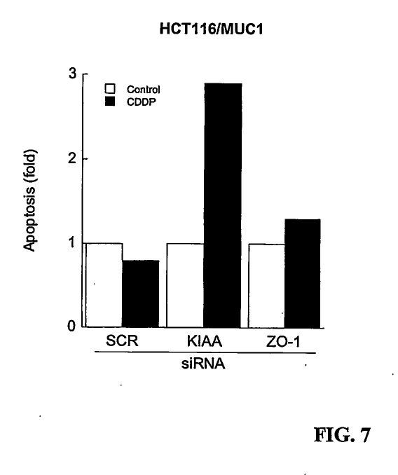

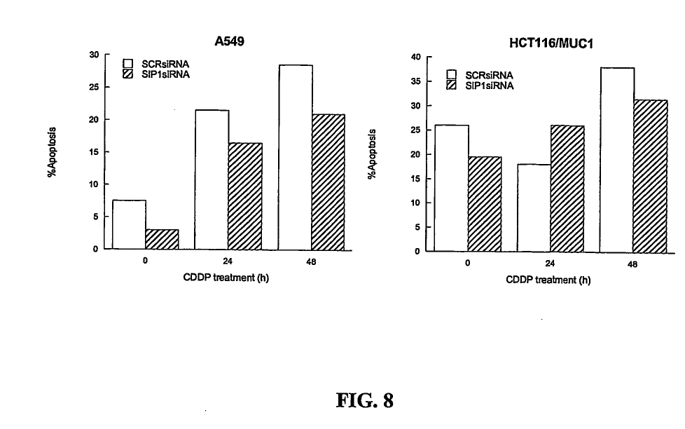

Results: In that MUC1 functions as anti-apoptotic protein, HCT116/vector cells were sensitive to apoptosis induced by CDDP (100 μM) while HCT116/MUC1 cells were relatively resistant to apoptosis. Transient transfections of HCT116/MUC1 cells with KIAA0751siRNA and LIM-MsiRNA were associated with increased apoptotic responses to CDDP (FIG. 5). Similar results were obtained with A549 cells that endogenously express MUC1 (FIG. 5). The apoptosis-sensitizing effect of KIAA0751siRNA was significantly greater than that of LIM-MsiRNA in HCT116/MUC1 cells. Importantly, KIAAO751siRNA did not sensitize cells to apoptosis in MUC1-negative HCT116/vector cells at either 10 μM or 100 μM CDDP, indicating that the observed apoptosis-sensitizing effect of KIAAO751siRNA is dependent on MUC1 (FIG. 6) and that the KIAA0751 protein is involved in the anti-apoptotic function of MUC1. Conversely, neither SIP1siRNA nor ZO1siRNA significantly affected CDDP-induced apoptosis in A549 and HCT116/MUC1 cells (FIG. 7 and FIG. 8).

The knock-down effects of siRNAs on ZO-1, KIAAO751 and SIP1 were determined by immunoblotting and these proteins were knocked-down by approximately 50-70%.

### Example 10

### Comparative Binding of MUC1 Carboxy-Terminal Isoforms

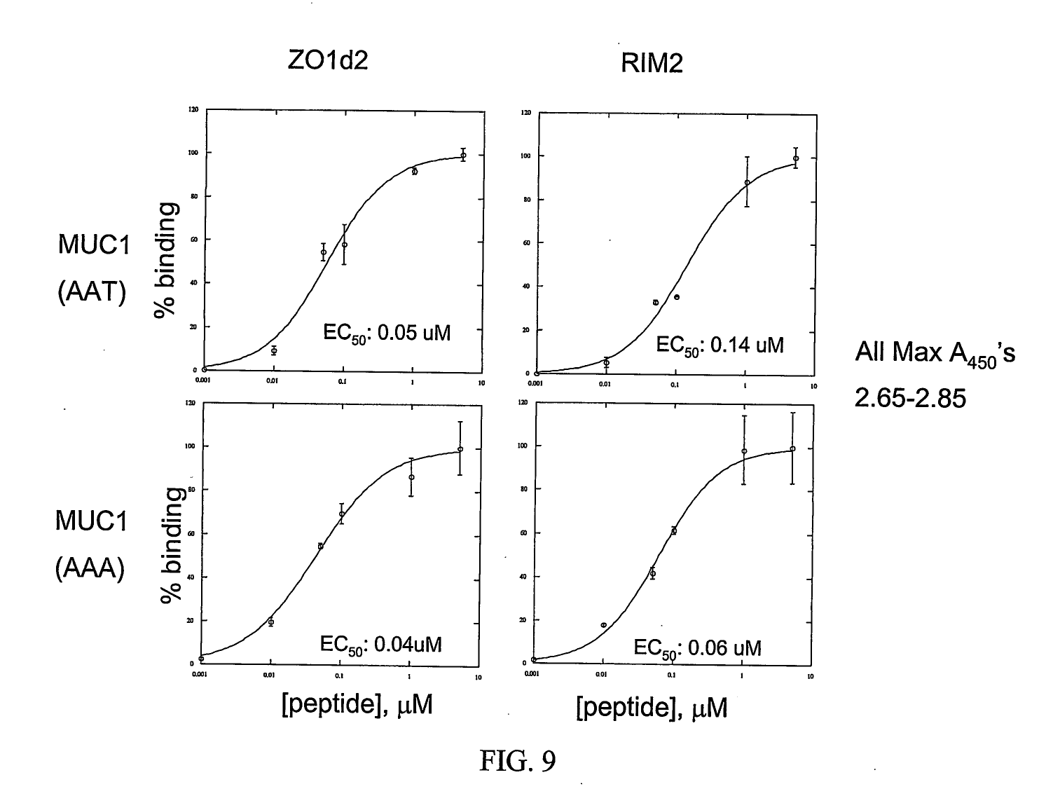

Using the modified ELISA described supra in Example 6, the effect of two variant carboxy-terminal MUC1 peptides were examined. Two MUC1 isoforms with an A/T substitutions at the fifth amino acid residue from the carboxy-terminal end have been reported in the literature, e.g., carboxy-terminal AAASANL disclosed in GenBank P15941 [gi:547937] and carboxy-terminal AATSANL disclosed in GenBank A35175 [gi:11385307]. Peptides were prepared consisting of the TAT sequence SEQ ID NO: 102 and the terminal nine amino acid residues of the relevant MUC1 sequence, i.e., YGRKKRRQRRRAVAATSANL (SEQ ID NO: 134) and YGRKKRRQRRRAVAAASANL (SEQ ID NO: 135) and titrated binding to RIM2 and ZO1 d2. As shown in FIG. 9, the two isoforms bind to RIM3 and ZO1 d2 with similar affinities.

### Example 11

### Comparative Binding of Ligands to PDZ Domains

Using the modified ELISA described supra in Example 6, RIM2, ZO1 d2, SIP1 d1 and Lim Mystique were titrated with three peptides consisting of 9 carboxy-terminal amino acid residues and TAT SEQ ID NO: 102, i.e., biotinylated peptides:

| | |
|---|---|
| YGRKKRRQRRRARGDRKRIV; | (SEQ ID NO: 136) |
| YGRKKRRQRRRQDEEEGIWA; | (SEQ ID NO: 137) |
| and | |
| YGRKKRRQRRRAVAATSINL. | (SEQ ID NO 138) |

As shown in Table 7, SEQ ID NO: 137 binds most tightly to RIM2, followed by SEQ ID NO: 136 and SEQ ID NO: 138. All three peptides bind SIP1 and Lim Mystique with lower affinity than the MUC1 derived sequence SEQ ID NO: 96 (cf Table 4, Example 7), while binding with greater affinity to RIM2 and ZO 1 d2, indicating greater selectivity for the later two PDZ domains than the MUC1 derived sequence. SEQ ID NO: 137 binds RIM2 more strongly than ZO 1 d2.

TABLE 7
EC₅₀ Values for PDZ Binding

| Peptide | RIM2 | ZO1 d2 | SIP1 | Lim Mys. |
|---|---|---|---|---|
| SEQ ID NO: 136 | 0.02 μM | 0.02 μM | >5 μM | >5 μM |
| SEQ ID NO: 137 | 0.005 μM | 0.05 μM | >5 μM | >5 μM |
| SEQ ID NO: 138 | 0.04 μM | 0.008 μM | >5 μM | >5 μM |

### Example 12

### Competitive Binding of Ligands to PDZ Domains

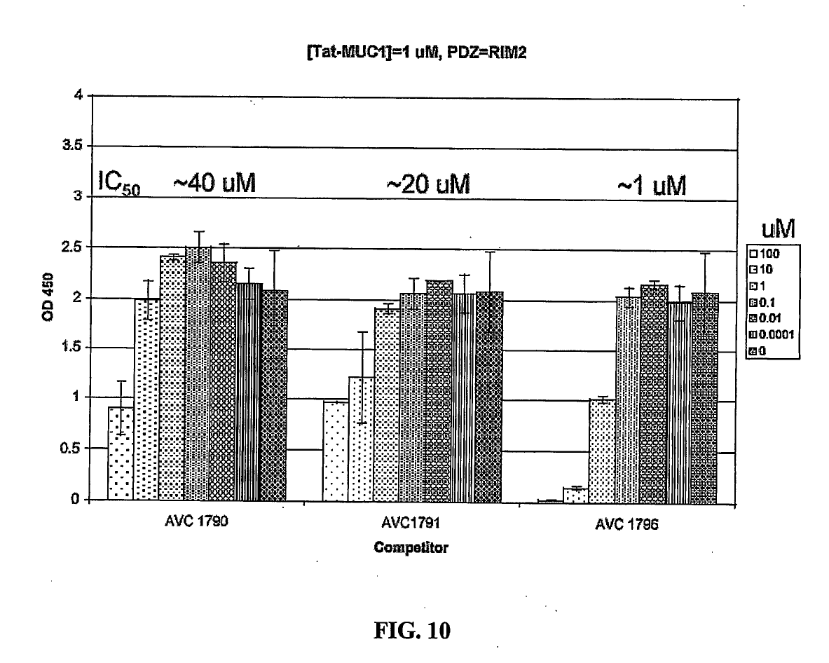

Using the modified ELISA described suprain Example 6, the ability of the peptides: YGRKKRRQRRRARGDRKRIV (SEQ ID NO: 136) (AVC 1796); YGRKKRRQRRRQDEEEGIWA (SEQ ID NO: 137) (AVC 1790); and YGRKKRRQRRRAVAATSINL (SEQ ID NO 138) (AVC 1791), to compete with the binding of the biotinylated TAT-MUC1 derived peptide YGRKKRRQRRRAVAATSANL (SEQ ID NO: 134) to PDZ domains. FIG. 10 shows that SEQ ID NO: 136 (AVC 1796) is the best competitive inhibitor for biotinylated SEQ ID NO: 134 (TAT-MUC1) binding to RIM2, though SEQ ID NO: 137 (AVC 1790) has a lower EC₅₀ for binding to RIM2 (cf Example 10). Similar experiments for binding to ZO1 d2 indicated that SEQ ID NO: 136 (AVC 1796) can also compete for binding to ZO1 d2 while SEQ ID NO: 137 (AVC 1790) is only a relatively weak competitor for ZO1 d2. Self-competition experiments indicated that SEQ ID NO: 137 (AVC 1790) acts the most like a traditional competitive inhibitor of the three peptides tested.

### Example 13

### Matrix Profile of Inhibitors

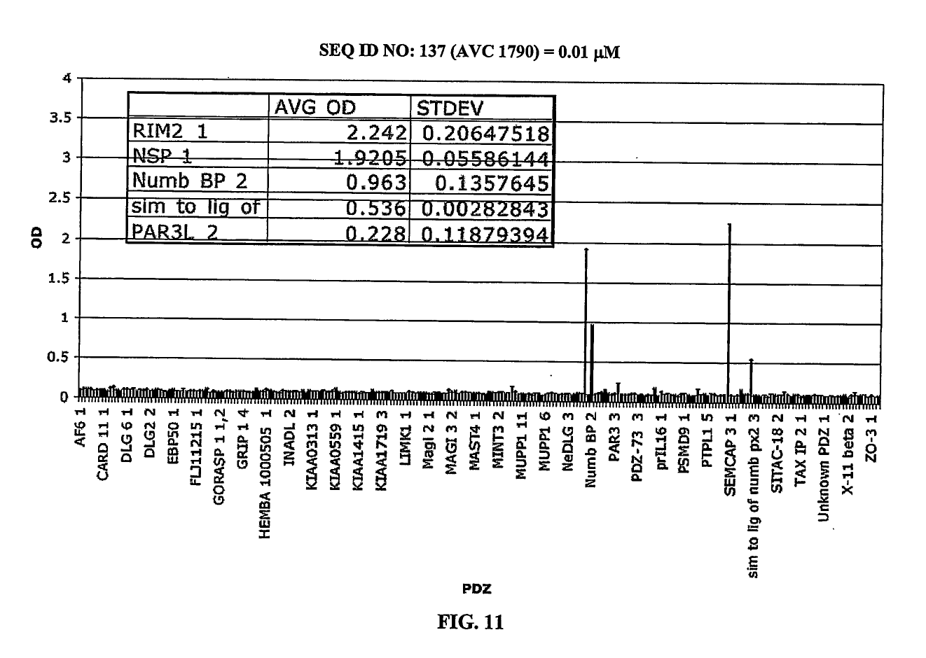

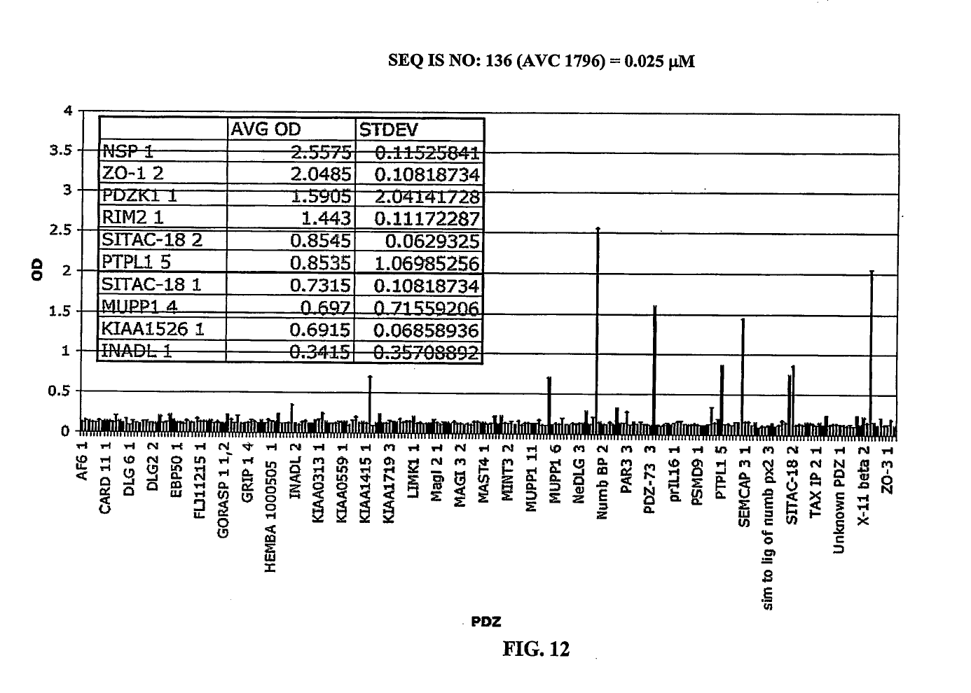

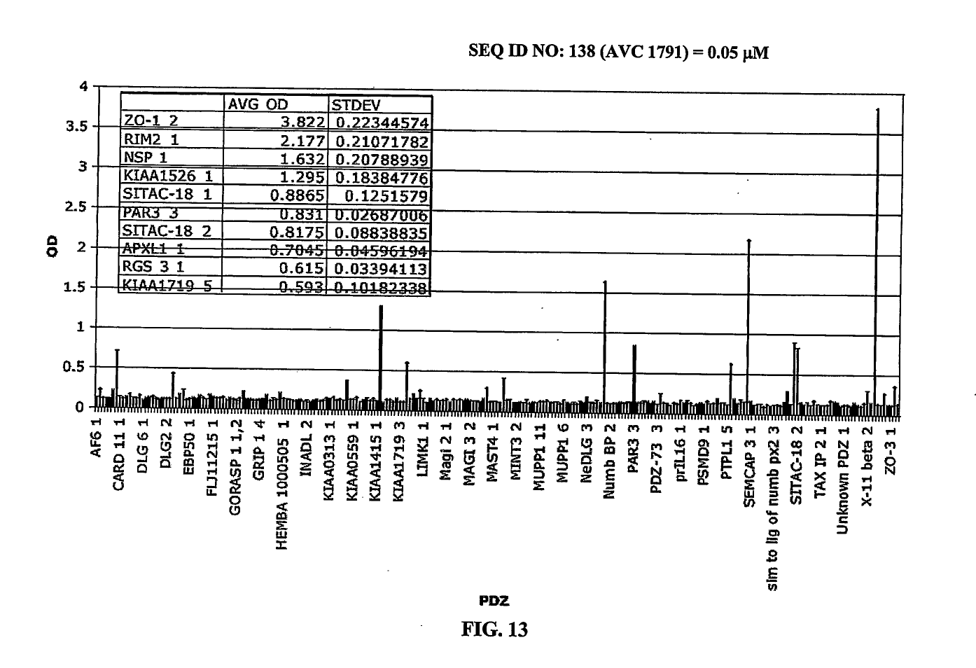

The biotinylated peptides SEQ ID NO: 136 (AVC 1796), SEQ ID NO: 137 (AVC 1790), and SEQ ID NO: 138 (AVC 1791), were screened for binding to PDZ domains as described in Example 7. The results, shown in FIGS. 11, 12 and 13, represent the absorbance and standard deviation of interactions of higher relative strength. The data in FIG. 13 for PDZK1, PTPL1 d5, MUPP1 d4 and INADL d1 have high standard deviations and thus require further verification to validate intensity of binding.

### Example 14

### Identification of Inhibitors of the MUC1-RIM2 Interaction

Using the modified ELISA described supra in Example 7, the binding to the RIM2 PDZ domain of the biotinylated peptide sequences listed in Table 8 were examined. The biotinylated 20-mer amino acid peptides were added at varying concentrations (0.001 μM to 10 μM) to the plated GST-RIM2 PDZ domain. Relative EC₅₀ values were calculated from a curve fit of the data for each interaction.

TABLE 8
Peptide binding to PDZ domain 1 of RIM2

| | | |
|---|---|---|
| | Relative | |
| Peptide (designation) | EC50 | SEQ ID NO: |

| | | |
|---|---|---|
| YGRKKRRQRRRAVAATSANL | 0.065 | SEQ ID NO: 134 |
| YGRKKRRQRRRARGDRKRIV | 0.02 | SEQ ID NO: 136 |
| (AVC#1796) | | |
| YGRKKRRQRRRQDEEEGIWA | 0.005 | SEQ ID NO: 137 |
| (AVC#1790) | | |
| YGRKKRRQRRRAVAATSINL | 0.04 | SEQ ID NO: 138 |
| (AVC#1791) | | |
| YGRKKRRQRRRAVAATYSNL | 0.6 | SEQ ID NO: 139 |
| (AVC#1793) | | |
| YGRKKRRQRRRARGDRKRWA | 0.007 | SEQ ID NO: 140 |
| (AVC#1821) | | |
| YGRKKRRQRRRARGDRKRWL | 0.008 | SEQ ID NO: 141 |
| (AVC#1822) | | |
| YGRKKRRQRRRARSDRGIWA | <0.01 | SEQ ID NO: 142 |
| (AVC#1823) | | |
| YGRKKRRQRRRAVAATGIWA | <0.01 | SEQ ID NO: 143 |
| (AVC#1827) | | |
| YGRKKRRQRRRQDEEETIWA | 0.24 | SEQ ID NO: 144 |
| (AVC#1828) | | |
| YGRKKRRQRRRARSDRTIWA | <0.01 | SEQ ID NO: 145 |
| (AVC#1829) | | |
| YGRKKRRQRRRARSDRTIIA | 0.013 | SEQ ID NO: 146 |
| (AVC#1830) | | |
| YGRKKRRQRRRARSDRKRIA | 0.045 | SEQ ID NO: 147 |
| (AVC#1831) | | |
| YGRKKRRQRRRSRTDRKYWA | <0.01 | SEQ ID NO: 148 |
| (AVC#1832) | | |
| YGRKKRRQRRRQDEEEGIWS | 0.05 | SEQ ID NO: 149 |
| (AVC#1833) | | |
| YGRKKRRQRRRSRTVREIWA | <0.01 | SEQ ID NO: 150 |
| (AVC#1834) | | |
| YGRKKRRQRRRSVTSTSINL | 0.09 | SEQ ID NO: 151 |
| (AVC#1835) | | |
| YGRKKRRQRRRARGDRKIRV | 0.01 | SEQ ID NO: 152 |
| (AVC#1836) | | |
| YGRKKRRQRRRARTDRKVEV | 0.04 | SEQ ID NO: 153 |
| (AVC#1837) | | |
| YGRKKRRQRRRARGDRKYIV | 0.013 | SEQ ID NO: 154 |
| (AVC#1838) | | |
| YGRKKRRQRRRSRTDRKYQI | 0.022 | SEQ ID NO: 155 |
| (AVC#1839) | | |
| YGRKKRRQRRRARGDVRLML | ~0.03 | SEQ ID NO: 156 |
| (AVC#1840) | | |
| YGRKKRRQRRRARGDRKVPV | 0.045 | SEQ ID NO: 157 |
| (AVC#1841) | | |
| YGRKKRRQRRRQDERRLIVL | 0.078 | SEQ ID NO: 158 |
| (AVC#1842) | | |
| YGRKKRRQRRRARGDRLVSL | 0.068 | SEQ ID NO: 159 |
| (AVC#1843) | | |
| YGRKKRRQRRRARGTRLVWV | <0.01 | SEQ ID NO: 160 |
| (AVC#1844) | | |
| YGRKKRRQRRRARGDRYRIV | 0.038 | SEQ ID NO: 161 |
| (AVC#1845) | | |
| YGRKKRRQRRRSRTDRLEYV | 0.01 | SEQ ID NO: 162 |
| (AVC#1846) | | |
| YGRKKRRQRRRARGDRLEIV | 0.132 | SEQ ID NO: 163 |
| (AVC#1847) | | |
| YGRKKRRQRRRARGDRTIIY | 0.03 | SEQ ID NO: 164 |
| (AVC#1848) | | |
| YGRKKRRQRRRARGDRRRIV | 0.037 | SEQ ID NO: 165 |
| (AVC#1849) | | |
| YGRKKRRQRRRARGDRKKIV | 0.047 | SEQ ID NO: 166 |
| (AVC#1850) | | |
| YGRKKRRQRRRARSDRKRIV | 0.047 | SEQ ID NO: 167 |
| (AVC#1851) | | |
| YGRKKRRQRRRKNKDKEYYV | 0.013 | SEQ ID NO: 168 |
| (AVC#1852) | | |
| YGRKKRRQRRRGMTSSSSVV | 0.135 | SEQ ID NO: 169 |
| (AVC#1853) | | |
| YGRKKRRQRRRARGRRETWV | <0.01 | SEQ ID NO: 170 |
| (AVC#1854) | | |
| YGRKKRRQRRRQDERVETRV | 0.88 | SEQ ID NO: 171 |
| (AVC#1855) | | |
| YGRKKRRQRRRLQRRRETQV | 0.033 | |
| (AVC#1856) | | |

### Example 15

### Sensitization of Human Cancer Cells to Chemotherapeutic Agents by Inhibitor Peptides

The effects of peptide inhibitors of the MUC1-RIM2 interaction on sensitizing MUC1-expressing human cancer cells to chemotherapeutic agents is investigated. Suitable human cancer cells include MUC1 transfected HCT116 cells (and vector control cells) and human non-small cell lung cancer A549 cells that endogenously express MUC1. HCT116 cells and A549 cells are grown in Dulbecco's modified Eagle's medium (DMEM) and RPMI1640 medium, respectively, in a humidified 5% CO₂ atmosphere at 37° C. Media is supplemented with 10% fetal bovine serum (FBS), 100 units/ml of penicillin and 100 μg/ml of streptomycin. HCT116 cells are transfected with pIRES-puro2 or pIRES-puro2-MUC1 as described (Li et al., 2001(a)) and stable transfectants are selected in the presence of 0.4 μg/ml puromycin (Caliochem-Novabiochem).

Cancer cells are incubated with inhibitor peptides comprising an internalizing peptide sequence, including SEQ ID NO: 108 or SEQ ID NO: 119, and an inhibitor sequence, including SEQ ID NO: 134 through SEQ ID NO: 171. Suitable controls are also run in parallel. Subsequently, cells are treated with 0, 10 or 100 μM cisplatin (CDDP, Sigma) for 24 hr to induce apoptosis. Apoptotic cells are quantified by analysis of sub-G1 DNA content. Cells are harvested, washed, with PBS, fixed with 75% ethanol, and incubated in PBS containing 200 μg/ml RNase A (Qiagen) for 15 min at 37° C. Cells were then stained with 50 μg/ml propidium iodide (Boeringer Manheim) for 30 min at room temperature in the dark. DNA content was analyzed by flow cytometry (EPICS XL-MCL, Coulter Corp.).

### Example 16

### Human Cancer Cell in In Vivo Xenograft Models

The antitumor effect of inhibitor peptides, as described in Example 14, are assessed against MUC1-expressing human cancer cell xenograft tumor models. Suitable tumor cells include MUC1 transfected human colon cancer HCT116 cells (and vector control cells), human breast cancer ZR-75 cells and human non-small cell lung cancer A549 cells. Human tumors are implanted subcutaneously into the flanks of nude mice. As the tumors reach a predetermined size of approximately 100 mm³, the mice are randomized into therapy groups. Inhibitor peptides and suitable controls are administered by IV injection or intraperitoneal injection for a suitable time period, e.g., 5 daily doses at suitable does levels, e.g., maximum tolerated dose (MTD), ½ MTD, ¼ MTD, or other suitable dose if an MTD is not established. Mean tumor volumes are determined three times per week. Tumor volume is determined by caliper measurements (mm) and using the formula for an ellipsoid sphere: L×W²/2=mm³, where L is the length in mm and W is the width in mm. The formula is also used to calculate tumor weight (mg), assuming unit density (1 mm³=1 mg). The study is terminated when the tumor volumes in the control group(s) reach approximately 2000 mm³. The time to reach evaluation size for the tumor of each animal is used to calculate the overall delay in the growth of the median tumor (T-C).

The present invention has been shown by both description and examples. The Examples are only examples and cannot be construed to limit the scope of the invention. One of ordinary skill in the art will envision equivalents to the inventive process described by the following claims that are within the scope and spirit of the claimed invention.

### REFERENCES

The following references, to the extent that they provide exemplary procedural or other details supplementary to those set forth herein, are specifically incorporated herein by reference.
Banerjee, Biopolymers, 39:769-777, 1996.
Barry & Sharkey, Hum. Pathol., 16:225-7, 1985.
Beusen, Biopolymers, 36:181-200, 1995.
Bird et al., Science, 242:423-426, 1988.
Brunner et al., Nature, 385:829-833, 1997.
Chaudhary et al., Proc. Nat'l. Acad. Sci. U.S.A., 87 :1066-1070, 1990.
Console et al., J. Biol. Chem., 278 :35109-35114, 2003.
Daniels & Reynolds, Mol. Cell. Biol., 19:3614-23, 1999.
Dawson et al., Science, 266:776, 1994.
Derossi et al., J. Biol. Chem., 269:10444-10450, 1994.
Derossi et al., J. Biol. Chem., 271:18188-18193, 1996.
Deveraux & Reed, Genes Dev., 13:239-252, 1999.
Doyle, Cell, 85: 1067-76, 1996.
Elliot & O'Hare, Cell, 88:223-233, 1997. Morris et al., Nucelic Acid Res., 25 :2730-2736, 1997.
Elmquist et al., Exp. Cell Res., 269 :237-244, 2001.
Eubanks et al., in: Peptides, Chemistry and Biology, Garland Marshall (ed.), ESCOM,
Leiden, pp. 566-69, 1988.
Faccio et al., J. Biol. Chem., 28:2581-2588, 2000.
Feigl, J. Amer. Chem. Soc., 108:181-182, 1986.
Finn et al., Immunol. Rev., 145:61-89, 1995.
Frankel and Pabo, Cell, 55:1189-1193, 1989.
Futaki et al., J. Biol. Chem., 276 :5836-5840, 2001.
Green and Loewenstein, Cell 55:1179-1188, 1989.
Harrison, Cell, 86:341-343, 1996.
Higashijima et al., J. Biol. Chem., 265:14176, 1990.
Higgins, J. Pept. Res., 50:421-435, 1997.
Ho et al., Cancer Res., 61:474-477, 2001.
Hruby, Biopolymers, 43:219-266, 1997.
Huston et al., Proc. Nat'l. Acad. Sci. U.S.A., 85 :5979-5883, 1988.
Itzkowitz et al., Cancer, 66:1960-6, 1990.
Jawhari et al., J. Pathol., 189:180-85, 1999.
Jin et al., Genes Dev., 17:359-367, 2003.
Kahn, J. Amer. Chem. Soc., 110:1638-1639, 1988.
Kahn, J. Molec. Recognition, 1:75-79, 1988a.
Karvinen et al., J. Biomol. Screen., 7P:223-231, 2002.
Kemp, Tet. Lett., 29:5057-5060, 1988.
Kufe et al., Hybridoma, 3:223-232, 1984.
Kuppuswamy et al., Nucl. Acids Res., 17:3551-3561, 1989.
Li et al., Mol Cell Biol., 18:7216-24, 1998.
Li et al., J. Biol. Chem., 276:6061-64, 2001.
Li et al., J. Biol. Chem., 276:35239-42, 2001(a).
Li & Kufe, Biochem, Biophys. Res. Commun., 281, 440-43, 2001.
Lin et al., J. Biol. Chem., 270 :14255-14258, 1995.
Liu et al., Proc. Nat'l Acad. Sci. U.S.A., 93 :11819-11824, 1996.
Martins, Cell Death Diff, 9:699-701, 2002.
Mi et al., Mol. Ther., 2 :339-347, 2000.
Molenaar et al., Cell, 86:391-399, 1996.
Nagai, Tet. Lett., 26:647-650, 1985.
Niethammer et al., Neuron., 20:693-707, 1989. Novak & Dedhar, Cell Mol. Life. Sci., 523-37, 1999.
Oehlke et al., Biochim. Biophys. Acta., 1414 :127-139, 1998.
Perey et al., Cancer Res., 52:2563-68, 1992.
Perez et al., J. Cell. Sci., 102:717-722, 1992.
Pescarolo et al., FASEB J., 15 :31-33, 2001.
Pooga et al., Nature Biotech., 16 :857-861, 1998.
Reddish et al., Cancer Immunol. Immunother., 42:303-9, 1996.
Reynolds et al., Mol. Cell. Biol., 9:629-38, 1989.
Reynolds et al., Mol. Cell. Biol., 14:8333-41, 1994.
Rousselle et al., Mol. Pharmacol., 57 :679-686, 2000.
Ruben et al., J. Virol., 63:1-8, 1989.
Schneider et al, Nat. Biotech., 17:170-5, 1998.
Schultz et al., Science, 275:73-77, 1997.
Shimazui et al., Cancer Res., 56:4154-58, 1996.
Smith, J. Amer. Chem. Soc., 114:10672-10674, 1992.
Smith & Waterman, Adv. Appl. Math., 2:482-489, 1981.
Snyder et al., PLOS Biology., 2 :186-193, 2004.
Songyang et al., Science, 275:73-7, 1997.
Soomets et al., Biochim. Biophys. Acta, 1467:165-176, 2000.
Strouss & Decker, Crit. Rev. Biochem., Mol. Biol., 27:57-92, 1992.
Subbarao et al., Biochemistry, 26:2964, 1987.
Sujatha et al., J. Biomol. Screen, 7:317-323, 2002.
Suzuki et al., J. Biol. Chem., 277 :2437-2443, 2002.
Takeichi, Annu. Rev. Biochem., 59:237-52, 1990.
Torchilin & Levchenko, Curr. Protein Pept. Sci., 4:133-140, 2003.
Tseng et al., Mol. Pharmcaol., 62:864-872, 2002.
Ulysse, et al., J. Am. Chem. Soc., 117:8466-8467, 1995.
Vives et al., J. Biol. Chem., 272 :16010-16017, 1997.
Wender et al., Proc. Nat'l. Acad. Sci., U.S.A., 97:13003-13008, 2000.
Willimas et al., J. Biol. Chem., 272:22349-22354, 1997.
Yamamoto et al., J. Biol. Chem., 272:12492-94, 1997.

1. A method of inhibiting the binding of the cytoplasmic domain of MUC1 to a PDZ domain, comprising contacting said PDZ domain with an effective amount of an agent that competes with the binding of the C-terminal region of said cytoplasmic domain of MUC1 with said PDZ domain.

2. The method of claim 1, wherein said PDZ domain is ZO-1 d2, SIP1 d1, LIM MYSTIQUE, AIPC, KIAA0751, MAST2, PRIL-16 d1, GRIP2 d5, SITAC 18, NSP or KIAA1526 d1.

3. The method of claim 1, wherein said agent that competes with binding of said C-terminal region of cytoplasmic domain of MUC1 with said PDZ domain is a peptide of the formula X¹-aa²-aa¹-aa⁰, wherein aa⁰ is a hydrophobic aliphatic amino acid residue or a hydrophobic aromatic amino acid residue, aa² is a hydrophobic aliphatic amino acid residue, hydrophobic aromatic amino acid residue, polar amino acid residue, basic amino acid residue or an acidic amino acid residue, aa¹ is an amino acid residue and X¹ is a sequence of 0 to 50 amino acid residues.

4. The method of claim 3, wherein aa⁰ is V, L, A, I, S or Y and aa² is V, L, A, I, F, Y, W, Q, N, S, T, R, K, D or E.

5. The method of claim 3, wherein aa²-aa¹-aa⁰ is a sequence selected from SEQ ID NO: 1 through SEQ ID NO: 40.

6. The method of claim 3, wherein the carboxy-terminus of said peptide of formula X¹-aa²-aa¹-aa⁰ comprises the carboxy-terminal 4, 5 6, 7, 8 or 9 amino acid residues of a nine amino acid residue sequence selected from SEQ ID NO: 41 through SEQ ID NO: 94.

7. The method of claim 3, wherein the carboxy-terminus of said peptide of formula X¹-aa²-aa¹-aa⁰ comprises the carboxy-terminal 4, 5 6, 7, 8, 9, 10, 11, 12, 13, 14, 15, 16, 17, 18, 19 or 20 amino acid residues of SEQ ID NO: 95 or SEQ ID NO: 96.

8. The method of claim 3, wherein the amino terminus of X¹ comprises X²-X³, wherein X² is a transmembrane transporter peptide sequence and X³ is an optional linker sequence.

9. The method of claim 8, wherein X² is a sequence selected from SEQ ID NO 97 through SEQ ID NO: 127.

10. The method of claim 9, wherein X² is SEQ ID NO: 102, SEQ ID NO: 108 or SEQ ID NO: 119.

11. A method of inhibiting the binding of the cytoplasmic domain of MUC1 to one or more PDZ proteins within a MUC1 expressing cancer cell comprising contacting said MUC1 expressing cancer cell with an effective amount of an agent that competes with the binding of the C-terminal region of said cytoplasmic domain of MUC1 with said PDZ protein.

12. The method of claim 11, wherein one or more PDZ proteins is/are selected from the group consisting of ZO-1 d2, SIP1 d1, LIM MYSTIQUE, AIPC, KIAAO751, MAST2, and PRIL-16 d1.

13. The method of claim 11, wherein said agent that competes with binding of said C-terminal region of cytoplasmic domain of MUC1 with said one or more PDZ proteins is a peptide of the formula X¹-aa²-aa¹-aa⁰, wherein aa⁰ is a hydrophobic aliphatic amino acid residue, aa² is a hydrophobic aliphatic amino acid residue or a hydrophobic aromatic amino acid residue, hydrophobic aromatic amino acid residue, polar amino acid residue, basic amino acid residue or an acidic amino acid residue, aa¹ is an amino acid residue and X¹ is a sequence of 0 to 50 amino acid residues.

14. The method of claim 13, wherein aa⁰ is V, L, A, I, S or Y and aa² is V, L, A, I, F, Y, W, Q, N, S, T, R, K, D or E.

15. The method of claim 13, wherein aa²-aa¹-aa⁰ is a sequence selected from SEQ ID NO: 1 through SEQ ID NO: 40.

16. The method of claim 13, wherein the carboxy-terminus of said peptide of formula X¹-aa²-aa¹-aa⁰ comprises the carboxy-terminal 4, 5 6, 7, 8 or 9 amino acid residues of a nine amino acid residue sequence selected from SEQ ID NO: 41 through SEQ ID NO: 94.

17. The method of claim 13, wherein the carboxy-terminus of said peptide of formula X¹-aa²-aa¹-aa⁰ comprises the carboxy-terminal 4, 5 6, 7, 8, 9, 10, 11, 12, 13, 14, 15, 16, 17, 18, 19 or 20 amino acid residues of SEQ ID NO: 95 or SEQ ID NO: 96.

18. The method of claim 13, wherein the amino terminus of X¹ comprises X²-X³, wherein X² is a transmembrane transporter peptide sequence and X³ is an optional linker sequence.

19. The method of claim 18, wherein X² is a sequence selected from SEQ ID NO: 97 through SEQ ID NO: 127.

20. The method of claim 19, wherein X² is SEQ ID NO: 102, SEQ ID NO: 108 or SEQ ID NO: 119.

21. A method of enhancing the sensitivity of MUC1-expressing cancer cells to chemotherapeutic agents comprising contacting said MUC1-expressing cancer with an effective amount of a peptide of the formula X¹-aa²-aa¹-aa⁰, wherein aa⁰ is a hydrophobic aliphatic amino acid residue or a hydrophobic aromatic amino acid residue, aa² is a hydrophobic aliphatic amino acid residue, hydrophobic aromatic amino acid residue, polar amino acid residue, basic amino acid residue or an acidic amino acid residue, aa¹ is an amino acid residue and X¹ is a sequence of 0 to 50 amino acid residues.

22. The method of claim 21, wherein aa⁰ is V, L, A, I, S or Y and aa² is V, L, A, I, F, Y, W, Q, N, S, T, R, K, D or E.

23. The method of claim 21, wherein aa²-aa¹-aa⁰ is a sequence selected from SEQ ID NO: 1 through SEQ ID NO: 40.

24. The method of claim 21, wherein the carboxy-terminus of said peptide of formula X¹-aa²-aa¹-aa⁰ comprises the carboxy-terminal 4, 5 6, 7, 8 or 9 amino acid residues of a nine amino acid residue sequence selected from SEQ ID NO: 41 through SEQ ID NO: 94.

25. The method of claim 21, wherein the carboxy-terminus of said peptide of formula X¹-aa²-aa¹-aa⁰ comprises the carboxy-terminal 4, 5 6, 7, 8, 9, 10, 11, 12, 13, 14, 15, 16, 17, 18, 19 or 20 amino acid residues of SEQ ID NO: 95 or SEQ ID NO: 96.

26. The method of claim 21, wherein the amino terminus of X¹ comprises X²-X³, wherein X² is a transmembrane transporter peptide sequence and X³ is an optional linker sequence.

27. The method of claim 26, wherein X² is a sequence selected from SEQ ID NO: 97 through SEQ ID NO: 127.

28. The method of claim 27, wherein X² is SEQ ID NO: 102, SEQ ID NO: 108 or SEQ ID NO: 119.

29. A method of killing MUC1-expressing cancer cells comprising contacting said MUC1-expressing cancer cells with an effective amount of a chemotherapeutic agent and an effective amount of a peptide of the formula X¹-aa²-aa¹-aa⁰, wherein aa⁰ is a hydrophobic aliphatic amino acid residue or a hydrophobic aromatic amino acid residue, aa² is a hydrophobic aliphatic amino acid residue, hydrophobic aromatic amino acid residue, polar amino acid residue, basic amino acid residue or an acidic amino acid residue, aa¹ is an amino acid residue and X¹ is a sequence of 0 to 50 amino acid residues.

30. The method of claim 29, wherein said chemotherapeutic agent is a DNA-interactive agent, a tubulin interactive agent, and an antimetabolite chemotherapeutic agent.

31. The method of claim 29, wherein aa⁰ is V, L, A, I, S or Y and aa² is V, L, A, I, F, Y, W, Q, N, S, T, R, K, D or E.

32. The method of claim 29, wherein aa²-aa¹-aa⁰ is a sequence selected from SEQ ID NO: 1 through SEQ ID NO: 40.

33. The method of claim 29, wherein the carboxy-terminus of said peptide of formula X¹-aa²-aa¹-aa⁰ comprises the carboxy-terminal 4, 5 6, 7, 8 or 9 amino acid residues of a nine amino acid residue sequence selected from SEQ ID NO: 41 through SEQ ID NO: 94.

34. The method of claim 29, wherein the carboxy-terminus of said peptide of formula X¹-aa²-aa¹-aa⁰ comprises the carboxy-terminal 4, 5 6, 7, 8, 9, 10, 11, 12, 13, 14, 15, 16, 17, 18, 19 or 20 amino acid residues of SEQ ID NO: 95 or SEQ ID NO: 96.

35. The method of claim 29, wherein the amino terminus of X¹ comprises X²-X³, wherein X² is a transmembrane transporter peptide sequence and X³ is an optional linker sequence.

36. The method of claim 35, wherein X² is a sequence selected from SEQ ID NO: 97 through SEQ ID NO: 127.

37. The method of claim 36, wherein X² is SEQ ID NO: 98, SEQ BD NO: 104 or SEQ ID NO: 119.

38. A method of killing MUC1-expressing cancer cells comprising contacting said MUC1-expressing cancer cells with an effective amount of a chemotherapeutic agent and an effective amount of an agent that inhibits the binding of the carboxy-terminal of the cytoplasmic tail of MUC1 with the PDZ domain of KIAAO751.

39. The method of claim 38, wherein said chemotherapeutic agent is a DNA-interactive agent, a tubulin interactive agent, and an antimetabolite chemotherapeutic agent.

40. The method of claim 38, wherein said agent that inhibits the binding of the carboxy-terminal of the cytoplamsic tail of MUC1 with the PDZ domain of KIAA0751 is a peptide of the formula X¹-aa²-aa¹-aa⁰, wherein aa⁰ is a hydrophobic aliphatic amino acid residue or a hydrophobic aromatic acid residue, aa² is a hydrophobic aliphatic amino acid residue, hydrophobic aromatic amino acid residue, polar amino acid residue, basic amino acid residue or an acidic amino acid residue, aa¹ is an amino acid residue and X¹ is a sequence of 0 to 50 amino acid residues.

41. The method of claim 40, wherein aa⁰ is V, L, A, I, S or Y and aa² is V, L, A, I, F, Y, W, Q, N, S, T, R, K, D or E.

42. The method of claim 40, wherein aa²-aa¹-aa⁰ is a sequence selected from SEQ ID NO: 1 through SEQ ID NO: 40.

43. The method of claim 40, wherein the carboxy-terminus of said peptide of formula X¹-aa²-aa¹-aa⁰ comprises the carboxy-terminal 4, 5 6, 7, 8 or 9 amino acid residues of a nine amino acid residue sequence selected from SEQ ID NO: 41 through SEQ ID NO: 94.

44. The method of claim 40, wherein the carboxy-terminus of said peptide of formula X¹-aa²-aa¹-aa⁰ comprises the carboxy-terminal 4, 5 6, 7, 8, 9, 10, 11, 12, 13, 14, 15, 16, 17, 18, 19 or 20 amino acid residues of SEQ ID NO: 95 or SEQ ID NO: 96.

45. The method of claim 40, wherein the amino terminus of X¹ comprises X²-X³, wherein X² is a transmembrane transporter peptide sequence and X³ is an optional linker sequence.

46. The method of claim 45, wherein X² is a sequence selected from SEQ ID NO: 97 through SEQ ID NO: 127.

47. The method of claim 45, wherein X² is SEQ ID NO: 102, SEQ ID NO: 108 or SEQ ID NO: 119.


SEQUENCE LISTING (190 sequences)

SEQ ID NO: 1 - protein, 3 aa, Artificial Sequence
  DESCRIPTION OF ARTIFICIAL SEQUENCE  SYNTHETIC PEPTIDE
RIV

SEQ ID NO: 2 - protein, 3 aa, Artificial Sequence
  DESCRIPTION OF ARTIFICIAL SEQUENCE  SYNTHETIC PEPTIDE
LYI

SEQ ID NO: 3 - protein, 3 aa, Artificial Sequence
  DESCRIPTION OF ARTIFICIAL SEQUENCE  SYNTHETIC PEPTIDE
SVV

SEQ ID NO: 4 - protein, 3 aa, Artificial Sequence
  DESCRIPTION OF ARTIFICIAL SEQUENCE  SYNTHETIC PEPTIDE
AEV

SEQ ID NO: 5 - protein, 3 aa, Artificial Sequence
  DESCRIPTION OF ARTIFICIAL SEQUENCE  SYNTHETIC PEPTIDE
SQL

SEQ ID NO: 6 - protein, 3 aa, Artificial Sequence
  DESCRIPTION OF ARTIFICIAL SEQUENCE  SYNTHETIC PEPTIDE
SAA

SEQ ID NO: 7 - protein, 3 aa, Artificial Sequence
  DESCRIPTION OF ARTIFICIAL SEQUENCE  SYNTHETIC PEPTIDE
SDA

SEQ ID NO: 8 - protein, 3 aa, Artificial Sequence
  DESCRIPTION OF ARTIFICIAL SEQUENCE  SYNTHETIC PEPTIDE
SLV

SEQ ID NO: 9 - protein, 3 aa, Artificial Sequence
  DESCRIPTION OF ARTIFICIAL SEQUENCE  SYNTHETIC PEPTIDE
SGI

SEQ ID NO: 10 - protein, 3 aa, Artificial Sequence
  DESCRIPTION OF ARTIFICIAL SEQUENCE  SYNTHETIC PEPTIDE
SKV

SEQ ID NO: 11 - protein, 3 aa, Artificial Sequence
  DESCRIPTION OF ARTIFICIAL SEQUENCE  SYNTHETIC PEPTIDE
FYA

SEQ ID NO: 12 - protein, 3 aa, Artificial Sequence
  DESCRIPTION OF ARTIFICIAL SEQUENCE  SYNTHETIC PEPTIDE
TRV

SEQ ID NO: 13 - protein, 3 aa, Artificial Sequence
  DESCRIPTION OF ARTIFICIAL SEQUENCE  SYNTHETIC PEPTIDE
TTL

SEQ ID NO: 14 - protein, 3 aa, Artificial Sequence
  DESCRIPTION OF ARTIFICIAL SEQUENCE  SYNTHETIC PEPTIDE
TDV

SEQ ID NO: 15 - protein, 3 aa, Artificial Sequence
  DESCRIPTION OF ARTIFICIAL SEQUENCE  SYNTHETIC PEPTIDE
SDV

SEQ ID NO: 16 - protein, 3 aa, Artificial Sequence
  DESCRIPTION OF ARTIFICIAL SEQUENCE  SYNTHETIC PEPTIDE
YFI

SEQ ID NO: 17 - protein, 3 aa, Artificial Sequence
  DESCRIPTION OF ARTIFICIAL SEQUENCE  SYNTHETIC PEPTIDE
YYV

SEQ ID NO: 18 - protein, 3 aa, Artificial Sequence
  DESCRIPTION OF ARTIFICIAL SEQUENCE  SYNTHETIC PEPTIDE
ELV

SEQ ID NO: 19 - protein, 3 aa, Artificial Sequence
  DESCRIPTION OF ARTIFICIAL SEQUENCE  SYNTHETIC PEPTIDE
IWA

SEQ ID NO: 20 - protein, 3 aa, Artificial Sequence
  DESCRIPTION OF ARTIFICIAL SEQUENCE  SYNTHETIC PEPTIDE
ANL

SEQ ID NO: 21 - protein, 3 aa, Artificial Sequence
  DESCRIPTION OF ARTIFICIAL SEQUENCE  SYNTHETIC PEPTIDE
IIA

SEQ ID NO: 22 - protein, 3 aa, Artificial Sequence
  DESCRIPTION OF ARTIFICIAL SEQUENCE  SYNTHETIC PEPTIDE
RIA

SEQ ID NO: 23 - protein, 3 aa, Artificial Sequence
  DESCRIPTION OF ARTIFICIAL SEQUENCE  SYNTHETIC PEPTIDE
YWA

SEQ ID NO: 24 - protein, 3 aa, Artificial Sequence
  DESCRIPTION OF ARTIFICIAL SEQUENCE  SYNTHETIC PEPTIDE
IWS

SEQ ID NO: 25 - protein, 3 aa, Artificial Sequence
  DESCRIPTION OF ARTIFICIAL SEQUENCE  SYNTHETIC PEPTIDE
INL

SEQ ID NO: 26 - protein, 3 aa, Artificial Sequence
  DESCRIPTION OF ARTIFICIAL SEQUENCE  SYNTHETIC PEPTIDE
VEV

SEQ ID NO: 27 - protein, 3 aa, Artificial Sequence
  DESCRIPTION OF ARTIFICIAL SEQUENCE  SYNTHETIC PEPTIDE
VEV

SEQ ID NO: 28 - protein, 3 aa, Artificial Sequence
  DESCRIPTION OF ARTIFICIAL SEQUENCE  SYNTHETIC PEPTIDE
YIV

SEQ ID NO: 29 - protein, 3 aa, Artificial Sequence
  DESCRIPTION OF ARTIFICIAL SEQUENCE  SYNTHETIC PEPTIDE
YQI

SEQ ID NO: 30 - protein, 3 aa, Artificial Sequence
  DESCRIPTION OF ARTIFICIAL SEQUENCE  SYNTHETIC PEPTIDE
LML

SEQ ID NO: 31 - protein, 3 aa, Artificial Sequence
  DESCRIPTION OF ARTIFICIAL SEQUENCE  SYNTHETIC PEPTIDE
VPV

SEQ ID NO: 32 - protein, 3 aa, Artificial Sequence
  DESCRIPTION OF ARTIFICIAL SEQUENCE  SYNTHETIC PEPTIDE
IVL

SEQ ID NO: 33 - protein, 3 aa, Artificial Sequence
  DESCRIPTION OF ARTIFICIAL SEQUENCE  SYNTHETIC PEPTIDE
VSL

SEQ ID NO: 34 - protein, 3 aa, Artificial Sequence
  DESCRIPTION OF ARTIFICIAL SEQUENCE  SYNTHETIC PEPTIDE
VWV

SEQ ID NO: 35 - protein, 3 aa, Artificial Sequence
  DESCRIPTION OF ARTIFICIAL SEQUENCE  SYNTHETIC PEPTIDE
EYV

SEQ ID NO: 36 - protein, 3 aa, Artificial Sequence [sequence omitted]

SEQ ID NO: 37 - protein, 3 aa, Artificial Sequence
  DESCRIPTION OF ARTIFICIAL SEQUENCE  SYNTHETIC PEPTIDE
IIY

SEQ ID NO: 38 - protein, 3 aa, Artificial Sequence
  DESCRIPTION OF ARTIFICIAL SEQUENCE  SYNTHETIC PEPTIDE
KIV

SEQ ID NO: 39 - protein, 3 aa, Artificial Sequence
  DESCRIPTION OF ARTIFICIAL SEQUENCE  SYNTHETIC PEPTIDE
TWV

SEQ ID NO: 40 - protein, 3 aa, Artificial Sequence
  DESCRIPTION OF ARTIFICIAL SEQUENCE  SYNTHETIC PEPTIDE
TQV

SEQ ID NO: 41 - protein, 9 aa, Artificial Sequence
  DESCRIPTION OF ARTIFICIAL SEQUENCE  SYNTHETIC PEPTIDE
ARGDRKRIV

SEQ ID NO: 42 - protein, 9 aa, Artificial Sequence
  DESCRIPTION OF ARTIFICIAL SEQUENCE  SYNTHETIC PEPTIDE
TLASHQLYI

SEQ ID NO: 43 - protein, 9 aa, Artificial Sequence
  DESCRIPTION OF ARTIFICIAL SEQUENCE  SYNTHETIC PEPTIDE
GMTSSSSVV

SEQ ID NO: 44 - protein, 9 aa, Artificial Sequence
  DESCRIPTION OF ARTIFICIAL SEQUENCE  SYNTHETIC PEPTIDE
YGSPRYAEV

SEQ ID NO: 45 - protein, 9 aa, Artificial Sequence
  DESCRIPTION OF ARTIFICIAL SEQUENCE  SYNTHETIC PEPTIDE
WPPSSSSQL

SEQ ID NO: 46 - protein, 9 aa, Artificial Sequence
  DESCRIPTION OF ARTIFICIAL SEQUENCE  SYNTHETIC PEPTIDE
DDYDDISAA

SEQ ID NO: 47 - protein, 9 aa, Artificial Sequence
  DESCRIPTION OF ARTIFICIAL SEQUENCE  SYNTHETIC PEPTIDE
LKPPATSDA

SEQ ID NO: 48 - protein, 9 aa, Artificial Sequence
  DESCRIPTION OF ARTIFICIAL SEQUENCE  SYNTHETIC PEPTIDE
DKERLTSDA

SEQ ID NO: 49 - protein, 9 aa, Artificial Sequence
  DESCRIPTION OF ARTIFICIAL SEQUENCE  SYNTHETIC PEPTIDE
FRNETQSLV

SEQ ID NO: 50 - protein, 9 aa, Artificial Sequence
  DESCRIPTION OF ARTIFICIAL SEQUENCE  SYNTHETIC PEPTIDE
ALRASESGI

SEQ ID NO: 51 - protein, 9 aa, Artificial Sequence
  DESCRIPTION OF ARTIFICIAL SEQUENCE  SYNTHETIC PEPTIDE
LVEAQKSKV

SEQ ID NO: 52 - protein, 9 aa, Artificial Sequence
  DESCRIPTION OF ARTIFICIAL SEQUENCE  SYNTHETIC PEPTIDE
PTKQEEFYA

SEQ ID NO: 53 - protein, 9 aa, Artificial Sequence
  DESCRIPTION OF ARTIFICIAL SEQUENCE  SYNTHETIC PEPTIDE
FSRRPKTRV

SEQ ID NO: 54 - protein, 9 aa, Artificial Sequence
  DESCRIPTION OF ARTIFICIAL SEQUENCE  SYNTHETIC PEPTIDE
SSGHTSTTL

SEQ ID NO: 55 - protein, 9 aa, Artificial Sequence
  DESCRIPTION OF ARTIFICIAL SEQUENCE  SYNTHETIC PEPTIDE
NIKKIFTDV

SEQ ID NO: 56 - protein, 9 aa, Artificial Sequence
  DESCRIPTION OF ARTIFICIAL SEQUENCE  SYNTHETIC PEPTIDE
KMDSIESDV

SEQ ID NO: 57 - protein, 9 aa, Artificial Sequence
  DESCRIPTION OF ARTIFICIAL SEQUENCE  SYNTHETIC PEPTIDE
DSSRKEYFI

SEQ ID NO: 58 - protein, 9 aa, Artificial Sequence
  DESCRIPTION OF ARTIFICIAL SEQUENCE  SYNTHETIC PEPTIDE
KNKDKEYYV

SEQ ID NO: 59 - protein, 9 aa, Artificial Sequence
  DESCRIPTION OF ARTIFICIAL SEQUENCE  SYNTHETIC PEPTIDE
VTDHKTELV

SEQ ID NO: 60 - protein, 9 aa, Artificial Sequence
  DESCRIPTION OF ARTIFICIAL SEQUENCE  SYNTHETIC PEPTIDE
QDEEEGIWA

SEQ ID NO: 61 - protein, 9 aa, Artificial Sequence
  DESCRIPTION OF ARTIFICIAL SEQUENCE  SYNTHETIC PEPTIDE
AVAATSINL

SEQ ID NO: 62 - protein, 9 aa, Artificial Sequence
  DESCRIPTION OF ARTIFICIAL SEQUENCE  SYNTHETIC PEPTIDE
AVAATYSNL

SEQ ID NO: 63 - protein, 9 aa, Artificial Sequence
  DESCRIPTION OF ARTIFICIAL SEQUENCE  SYNTHETIC PEPTIDE
ARGDRKRWA

SEQ ID NO: 64 - protein, 9 aa, Artificial Sequence
  DESCRIPTION OF ARTIFICIAL SEQUENCE  SYNTHETIC PEPTIDE
ARGDRKRWL

SEQ ID NO: 65 - protein, 9 aa, Artificial Sequence
  DESCRIPTION OF ARTIFICIAL SEQUENCE  SYNTHETIC PEPTIDE
AVAATGIWA

SEQ ID NO: 66 - protein, 9 aa, Artificial Sequence
  DESCRIPTION OF ARTIFICIAL SEQUENCE  SYNTHETIC PEPTIDE
QDEEETIWA

SEQ ID NO: 67 - protein, 9 aa, Artificial Sequence
  DESCRIPTION OF ARTIFICIAL SEQUENCE  SYNTHETIC PEPTIDE
ARSDRTIWA

SEQ ID NO: 68 - protein, 9 aa, Artificial Sequence
  DESCRIPTION OF ARTIFICIAL SEQUENCE  SYNTHETIC PEPTIDE
ARSDRTIIA

SEQ ID NO: 69 - protein, 9 aa, Artificial Sequence
  DESCRIPTION OF ARTIFICIAL SEQUENCE  SYNTHETIC PEPTIDE
ARSDRKRIA

SEQ ID NO: 70 - protein, 9 aa, Artificial Sequence
  DESCRIPTION OF ARTIFICIAL SEQUENCE  SYNTHETIC PEPTIDE
SRTDRKYWA

SEQ ID NO: 71 - protein, 9 aa, Artificial Sequence
  DESCRIPTION OF ARTIFICIAL SEQUENCE  SYNTHETIC PEPTIDE
QDEEEGIWS

SEQ ID NO: 72 - protein, 9 aa, Artificial Sequence
  DESCRIPTION OF ARTIFICIAL SEQUENCE  SYNTHETIC PEPTIDE
SRTVREIWA

SEQ ID NO: 73 - protein, 9 aa, Artificial Sequence
  DESCRIPTION OF ARTIFICIAL SEQUENCE  SYNTHETIC PEPTIDE
SVTSTSINL

SEQ ID NO: 74 - protein, 9 aa, Artificial Sequence
  DESCRIPTION OF ARTIFICIAL SEQUENCE  SYNTHETIC PEPTIDE
ARGDRKIRV

SEQ ID NO: 75 - protein, 9 aa, Artificial Sequence
  DESCRIPTION OF ARTIFICIAL SEQUENCE  SYNTHETIC PEPTIDE
ARTDRKVEV

SEQ ID NO: 76 - protein, 9 aa, Artificial Sequence
  DESCRIPTION OF ARTIFICIAL SEQUENCE  SYNTHETIC PEPTIDE
ARGDRKYIV

SEQ ID NO: 77 - protein, 9 aa, Artificial Sequence
  DESCRIPTION OF ARTIFICIAL SEQUENCE  SYNTHETIC PEPTIDE
SRTDRKYQI

SEQ ID NO: 78 - protein, 9 aa, Artificial Sequence
  DESCRIPTION OF ARTIFICIAL SEQUENCE  SYNTHETIC PEPTIDE
ARGDVRLML

SEQ ID NO: 79 - protein, 9 aa, Artificial Sequence
  DESCRIPTION OF ARTIFICIAL SEQUENCE  SYNTHETIC PEPTIDE
ARGDRKVPV

SEQ ID NO: 80 - protein, 9 aa, Artificial Sequence
  DESCRIPTION OF ARTIFICIAL SEQUENCE  SYNTHETIC PEPTIDE
QDERRLIVL

SEQ ID NO: 81 - protein, 9 aa, Artificial Sequence
  DESCRIPTION OF ARTIFICIAL SEQUENCE  SYNTHETIC PEPTIDE
ARGDRLVSL

SEQ ID NO: 82 - protein, 9 aa, Artificial Sequence
  DESCRIPTION OF ARTIFICIAL SEQUENCE  SYNTHETIC PEPTIDE
ARGTRLVWV

SEQ ID NO: 83 - protein, 9 aa, Artificial Sequence
  DESCRIPTION OF ARTIFICIAL SEQUENCE  SYNTHETIC PEPTIDE
ARGDRYRIV

SEQ ID NO: 84 - protein, 9 aa, Artificial Sequence
  DESCRIPTION OF ARTIFICIAL SEQUENCE  SYNTHETIC PEPTIDE
SRTDRLEYV

SEQ ID NO: 85 - protein, 9 aa, Artificial Sequence
  DESCRIPTION OF ARTIFICIAL SEQUENCE  SYNTHETIC PEPTIDE
ARGDRLEIV

SEQ ID NO: 86 - protein, 9 aa, Artificial Sequence
  DESCRIPTION OF ARTIFICIAL SEQUENCE  SYNTHETIC PEPTIDE
ARGDRTIIY

SEQ ID NO: 87 - protein, 9 aa, Artificial Sequence
  DESCRIPTION OF ARTIFICIAL SEQUENCE  SYNTHETIC PEPTIDE
ARGDRRRIV

SEQ ID NO: 88 - protein, 9 aa, Artificial Sequence
  DESCRIPTION OF ARTIFICIAL SEQUENCE  SYNTHETIC PEPTIDE
ARGDRKKIV

SEQ ID NO: 89 - protein, 9 aa, Artificial Sequence
  DESCRIPTION OF ARTIFICIAL SEQUENCE  SYNTHETIC PEPTIDE
ARSDRKRIV

SEQ ID NO: 90 - protein, 9 aa, Artificial Sequence
  DESCRIPTION OF ARTIFICIAL SEQUENCE  SYNTHETIC PEPTIDE
KNKDKEYYV

SEQ ID NO: 91 - protein, 9 aa, Artificial Sequence
  DESCRIPTION OF ARTIFICIAL SEQUENCE  SYNTHETIC PEPTIDE
GMTSSSSVV

SEQ ID NO: 92 - protein, 9 aa, Artificial Sequence
  DESCRIPTION OF ARTIFICIAL SEQUENCE  SYNTHETIC PEPTIDE
ARGRRETWV

SEQ ID NO: 93 - protein, 9 aa, Artificial Sequence
  DESCRIPTION OF ARTIFICIAL SEQUENCE  SYNTHETIC PEPTIDE
QDERVETRV

SEQ ID NO: 94 - protein, 9 aa, Artificial Sequence
  DESCRIPTION OF ARTIFICIAL SEQUENCE  SYNTHETIC PEPTIDE
LQRRRETQV

SEQ ID NO: 95 - protein, 20 aa, Artificial Sequence
  DESCRIPTION OF ARTIFICIAL SEQUENCE  SYNTHETIC PEPTIDE
NGGSSLSYTNPAVAAASANL

SEQ ID NO: 96 - protein, 20 aa, Artificial Sequence
  DESCRIPTION OF ARTIFICIAL SEQUENCE  SYNTHETIC PEPTIDE
NGGSSLSYTNPAVAATSANL

SEQ ID NO: 97 - protein, 16 aa, Artificial Sequence
  DESCRIPTION OF ARTIFICIAL SEQUENCE  SYNTHETIC PEPTIDE
RQIKIWFQNRRMKWKK

SEQ ID NO: 98 - protein, 19 aa, Artificial Sequence
  DESCRIPTION OF ARTIFICIAL SEQUENCE  SYNTHETIC PEPTIDE
SGRQIKIWFQNRRMKWKKC

SEQ ID NO: 99 - protein, 16 aa, Artificial Sequence
  DESCRIPTION OF ARTIFICIAL SEQUENCE  SYNTHETIC PEPTIDE
RRWRRWWRRWWRRWRR

SEQ ID NO: 100 - protein, 16 aa, Artificial Sequence
  DESCRIPTION OF ARTIFICIAL SEQUENCE  SYNTHETIC PEPTIDE
KKWKMRRNQFWIKIQR

SEQ ID NO: 101 - protein, 16 aa, Artificial Sequence
  DESCRIPTION OF ARTIFICIAL SEQUENCE  SYNTHETIC PEPTIDE
KKWKMRRNQFWIKIQR

SEQ ID NO: 102 - protein, 11 aa, Artificial Sequence
  DESCRIPTION OF ARTIFICIAL SEQUENCE  SYNTHETIC PEPTIDE
YGRKKRRQRRR

SEQ ID NO: 103 - protein, 13 aa, Artificial Sequence
  DESCRIPTION OF ARTIFICIAL SEQUENCE  SYNTHETIC PEPTIDE
GRRKRRQRRRPPQ

SEQ ID NO: 104 - protein, 14 aa, Artificial Sequence
  DESCRIPTION OF ARTIFICIAL SEQUENCE  SYNTHETIC PEPTIDE
SGYGRKKRRQRRRC

SEQ ID NO: 105 - protein, 11 aa, Artificial Sequence
  DESCRIPTION OF ARTIFICIAL SEQUENCE  SYNTHETIC PEPTIDE
RRRQRRKKRGY

SEQ ID NO: 106 - protein, 11 aa, Artificial Sequence
  DESCRIPTION OF ARTIFICIAL SEQUENCE  SYNTHETIC PEPTIDE
YARAAARQARA

SEQ ID NO: 107 - protein, 9 aa, Artificial Sequence
  DESCRIPTION OF ARTIFICIAL SEQUENCE  SYNTHETIC PEPTIDE
RKKRRQRRR

SEQ ID NO: 108 - protein, 8 aa, Artificial Sequence
  DESCRIPTION OF ARTIFICIAL SEQUENCE  SYNTHETIC PEPTIDE
RRRRRRRR

SEQ ID NO: 109 - protein, 6 aa, Artificial Sequence
  DESCRIPTION OF ARTIFICIAL SEQUENCE  SYNTHETIC PEPTIDE
RRRRRR

SEQ ID NO: 110 - protein, 8 aa, Artificial Sequence
  DESCRIPTION OF ARTIFICIAL SEQUENCE  SYNTHETIC PEPTIDE
RRRRRRRR

SEQ ID NO: 111 - protein, 6 aa, Artificial Sequence
  DESCRIPTION OF ARTIFICIAL SEQUENCE  SYNTHETIC PEPTIDE
RRRQRR

SEQ ID NO: 112 - protein, 27 aa, Artificial Sequence
  DESCRIPTION OF ARTIFICIAL SEQUENCE  SYNTHETIC PEPTIDE
GWTLNSAGYLLGKINLKALAALAKKIL

SEQ ID NO: 113 - protein, 21 aa, Artificial Sequence
  DESCRIPTION OF ARTIFICIAL SEQUENCE  SYNTHETIC PEPTIDE
AGYLLGKINLKALAALAKKIL

SEQ ID NO: 114 - protein, 33 aa, Artificial Sequence
  DESCRIPTION OF ARTIFICIAL SEQUENCE  SYNTHETIC PEPTIDE
DAATATRGRSAASRPTERPRAPARSASRPRRVE

SEQ ID NO: 115 - protein, 27 aa, Artificial Sequence
  DESCRIPTION OF ARTIFICIAL SEQUENCE  SYNTHETIC PEPTIDE
GALFLGFLGAAGSTMGAWSQPKSKRKV

SEQ ID NO: 116 - protein, 18 aa, Artificial Sequence
  DESCRIPTION OF ARTIFICIAL SEQUENCE  SYNTHETIC PEPTIDE
RGGRLSYSRRRFSTSTGR

SEQ ID NO: 117 - protein, 16 aa, Artificial Sequence
  DESCRIPTION OF ARTIFICIAL SEQUENCE  SYNTHETIC PEPTIDE
VTVVLALGALAGVGVG

SEQ ID NO: 118 - protein, 16 aa, Artificial Sequence
  DESCRIPTION OF ARTIFICIAL SEQUENCE  SYNTHETIC PEPTIDE
AAVALLPAVLLALLAP

SEQ ID NO: 119 - protein, 18 aa, Artificial Sequence
  DESCRIPTION OF ARTIFICIAL SEQUENCE  SYNTHETIC PEPTIDE
KLALKLALKALKAALKLA

SEQ ID NO: 120 - protein, 15 aa, Artificial Sequence
  DESCRIPTION OF ARTIFICIAL SEQUENCE  SYNTHETIC PEPTIDE
RRRRNRTRRNRRRVR

SEQ ID NO: 121 - protein, 12 aa, Artificial Sequence
  DESCRIPTION OF ARTIFICIAL SEQUENCE  SYNTHETIC PEPTIDE
PIRRRKKLRRLK

SEQ ID NO: 122 - protein, 12 aa, Artificial Sequence
  DESCRIPTION OF ARTIFICIAL SEQUENCE  SYNTHETIC PEPTIDE
RRQRRTSKLMKR

SEQ ID NO: 123 - protein, 18 aa, Artificial Sequence
  DESCRIPTION OF ARTIFICIAL SEQUENCE  SYNTHETIC PEPTIDE
LLIILRRRIRKQAHAHSK

SEQ ID NO: 124 - protein, 7 aa, Artificial Sequence
  DESCRIPTION OF ARTIFICIAL SEQUENCE  SYNTHETIC PEPTIDE
PKKKRKV

SEQ ID NO: 125 - protein, 16 aa, Artificial Sequence
  DESCRIPTION OF ARTIFICIAL SEQUENCE  SYNTHETIC PEPTIDE
KRPAAIKKAGQAKKKK

SEQ ID NO: 126 - protein, 12 aa, Artificial Sequence
  DESCRIPTION OF ARTIFICIAL SEQUENCE  SYNTHETIC PEPTIDE
CMHIESLDSYTC

SEQ ID NO: 127 - protein, 12 aa, Artificial Sequence
  DESCRIPTION OF ARTIFICIAL SEQUENCE  SYNTHETIC PEPTIDE
CMYIEALDKYAC

SEQ ID NO: 128 - protein, 5 aa, Artificial Sequence
  DESCRIPTION OF ARTIFICIAL SEQUENCE  SYNTHETIC PEPTIDE
GGGGS

SEQ ID NO: 129 - protein, 14 aa, Artificial Sequence
  DESCRIPTION OF ARTIFICIAL SEQUENCE  SYNTHETIC PEPTIDE
EGKSSGSGSESKVD

SEQ ID NO: 130 - protein, 18 aa, Artificial Sequence
  DESCRIPTION OF ARTIFICIAL SEQUENCE  SYNTHETIC PEPTIDE
KESGSVSSEQLAQFRSLD

SEQ ID NO: 131 - protein, 72 aa, Artificial Sequence
  DESCRIPTION OF ARTIFICIAL SEQUENCE  SYNTHETIC PEPTIDE
CQCRRKNYGQLDIFPARDTYHPMSEYPTYHTHGRYVPPSSTDRSPYEKVSAGNGGSSLSYTNPAVAATSANL

SEQ ID NO: 132 - protein, 68 aa, Artificial Sequence
  DESCRIPTION OF ARTIFICIAL SEQUENCE  SYNTHETIC PEPTIDE
CQCRRKNYGQLDIFPARDTYHPMSEYPTYHTHGRYVPPSSTDRSPYEKVSAGNGGSSLSYTNPAVAAT

SEQ ID NO: 133 - protein, 7 aa, Artificial Sequence
  DESCRIPTION OF ARTIFICIAL SEQUENCE  SYNTHETIC PEPTIDE
AAASANL

SEQ ID NO: 134 - protein, 20 aa, Artificial Sequence
  DESCRIPTION OF ARTIFICIAL SEQUENCE  SYNTHETIC PEPTIDE
YGRKKRRQRRRAVAATSANL

SEQ ID NO: 135 - protein, 20 aa, Artificial Sequence
  DESCRIPTION OF ARTIFICIAL SEQUENCE  SYNTHETIC PEPTIDE
YGRKKRRQRRRAVAAASANL

SEQ ID NO: 136 - protein, 20 aa, Artificial Sequence
  DESCRIPTION OF ARTIFICIAL SEQUENCE  SYNTHETIC PEPTIDE
YGRKKRRQRRRARGDRKRIV

SEQ ID NO: 137 - protein, 20 aa, Artificial Sequence
  DESCRIPTION OF ARTIFICIAL SEQUENCE  SYNTHETIC PEPTIDE
YGRKKRRQRRRQDEEEGIWA

SEQ ID NO: 138 - protein, 20 aa, Artificial Sequence
  DESCRIPTION OF ARTIFICIAL SEQUENCE  SYNTHETIC PEPTIDE
YGRKKRRQRRRAVAATSINL

SEQ ID NO: 139 - protein, 20 aa, Artificial Sequence
  DESCRIPTION OF ARTIFICIAL SEQUENCE  SYNTHETIC PEPTIDE
YGRKKRRQRRRAVAATYSNL

SEQ ID NO: 140 - protein, 20 aa, Artificial Sequence
  DESCRIPTION OF ARTIFICIAL SEQUENCE  SYNTHETIC PEPTIDE
YGRKKRRQRRRARGDRKRWA

SEQ ID NO: 141 - protein, 20 aa, Artificial Sequence
  DESCRIPTION OF ARTIFICIAL SEQUENCE  SYNTHETIC PEPTIDE
YGRKKRRQRRRARGDRKRWL

SEQ ID NO: 142 - protein, 20 aa, Artificial Sequence
  DESCRIPTION OF ARTIFICIAL SEQUENCE  SYNTHETIC PEPTIDE
YGRKKRRQRRRARSDRGIWA

SEQ ID NO: 143 - protein, 20 aa, Artificial Sequence
  DESCRIPTION OF ARTIFICIAL SEQUENCE  SYNTHETIC PEPTIDE
YGRKKRRQRRRAVAATGIWA

SEQ ID NO: 144 - protein, 20 aa, Artificial Sequence
  DESCRIPTION OF ARTIFICIAL SEQUENCE  SYNTHETIC PEPTIDE
YGRKKRRQRRRQDEEETIWA

SEQ ID NO: 145 - protein, 20 aa, Artificial Sequence
  DESCRIPTION OF ARTIFICIAL SEQUENCE  SYNTHETIC PEPTIDE
YGRKKRRQRRRARSDRTIWA

SEQ ID NO: 146 - protein, 20 aa, Artificial Sequence
  DESCRIPTION OF ARTIFICIAL SEQUENCE  SYNTHETIC PEPTIDE
YGRKKRRQRRRARSDRTIIA

SEQ ID NO: 147 - protein, 20 aa, Artificial Sequence
  DESCRIPTION OF ARTIFICIAL SEQUENCE  SYNTHETIC PEPTIDE
YGRKKRRQRRRARSDRKRIA

SEQ ID NO: 148 - protein, 20 aa, Artificial Sequence
  DESCRIPTION OF ARTIFICIAL SEQUENCE  SYNTHETIC PEPTIDE
YGRKKRRQRRRSRTDRKYWA

SEQ ID NO: 149 - protein, 20 aa, Artificial Sequence
  DESCRIPTION OF ARTIFICIAL SEQUENCE  SYNTHETIC PEPTIDE
YGRKKRRQRRRQDEEEGIWS

SEQ ID NO: 150 - protein, 20 aa, Artificial Sequence
  DESCRIPTION OF ARTIFICIAL SEQUENCE  SYNTHETIC PEPTIDE
YGRKKRRQRRRSRTVREIWA

SEQ ID NO: 151 - protein, 20 aa, Artificial Sequence
  DESCRIPTION OF ARTIFICIAL SEQUENCE  SYNTHETIC PEPTIDE
YGRKKRRQRRRSVTSTSINL

SEQ ID NO: 152 - protein, 20 aa, Artificial Sequence
  DESCRIPTION OF ARTIFICIAL SEQUENCE  SYNTHETIC PEPTIDE
YGRKKRRQRRRARGDRKIRV

SEQ ID NO: 153 - protein, 20 aa, Artificial Sequence
  DESCRIPTION OF ARTIFICIAL SEQUENCE  SYNTHETIC PEPTIDE
YGRKKRRQRRRARTDRKVEV

SEQ ID NO: 154 - protein, 20 aa, Artificial Sequence
  DESCRIPTION OF ARTIFICIAL SEQUENCE  SYNTHETIC PEPTIDE
YGRKKRRQRRRARGDRKYIV

SEQ ID NO: 155 - protein, 20 aa, Artificial Sequence
  DESCRIPTION OF ARTIFICIAL SEQUENCE  SYNTHETIC PEPTIDE
YGRKKRRQRRRSRTDRKYQI

SEQ ID NO: 156 - protein, 20 aa, Artificial Sequence
  DESCRIPTION OF ARTIFICIAL SEQUENCE  SYNTHETIC PEPTIDE
YGRKKRRQRRRARGDVRLML

SEQ ID NO: 157 - protein, 20 aa, Artificial Sequence
  DESCRIPTION OF ARTIFICIAL SEQUENCE  SYNTHETIC PEPTIDE
YGRKKRRQRRRARGDRKVPV

SEQ ID NO: 158 - protein, 20 aa, Artificial Sequence
  DESCRIPTION OF ARTIFICIAL SEQUENCE  SYNTHETIC PEPTIDE
YGRKKRRQRRRQDERRLIVL

SEQ ID NO: 159 - protein, 20 aa, Artificial Sequence
  DESCRIPTION OF ARTIFICIAL SEQUENCE  SYNTHETIC PEPTIDE
YGRKKRRQRRRARGDRLVSL

SEQ ID NO: 160 - protein, 20 aa, Artificial Sequence
  DESCRIPTION OF ARTIFICIAL SEQUENCE  SYNTHETIC PEPTIDE
YGRKKRRQRRRARGTRLVWV

SEQ ID NO: 161 - protein, 20 aa, Artificial Sequence
  DESCRIPTION OF ARTIFICIAL SEQUENCE  SYNTHETIC PEPTIDE
YGRKKRRQRRRARGDRYRIV

SEQ ID NO: 162 - protein, 20 aa, Artificial Sequence
  DESCRIPTION OF ARTIFICIAL SEQUENCE  SYNTHETIC PEPTIDE
YGRKKRRQRRRSRTDRLEYV

SEQ ID NO: 163 - protein, 20 aa, Artificial Sequence
  DESCRIPTION OF ARTIFICIAL SEQUENCE  SYNTHETIC PEPTIDE
YGRKKRRQRRRARGDRLEIV

SEQ ID NO: 164 - protein, 20 aa, Artificial Sequence
  DESCRIPTION OF ARTIFICIAL SEQUENCE  SYNTHETIC PEPTIDE
YGRKKRRQRRRARGDRTIIY

SEQ ID NO: 165 - protein, 20 aa, Artificial Sequence
  DESCRIPTION OF ARTIFICIAL SEQUENCE  SYNTHETIC PEPTIDE
YGRKKRRQRRRARGDRRRIV

SEQ ID NO: 166 - protein, 20 aa, Artificial Sequence
  DESCRIPTION OF ARTIFICIAL SEQUENCE  SYNTHETIC PEPTIDE
YGRKKRRQRRRARGDRKKIV

SEQ ID NO: 167 - protein, 20 aa, Artificial Sequence
  DESCRIPTION OF ARTIFICIAL SEQUENCE  SYNTHETIC PEPTIDE
YGRKKRRQRRRARSDRKRIV

SEQ ID NO: 168 - protein, 20 aa, Artificial Sequence
  DESCRIPTION OF ARTIFICIAL SEQUENCE  SYNTHETIC PEPTIDE
YGRKKRRQRRRKNKDKEYYV

SEQ ID NO: 169 - protein, 20 aa, Artificial Sequence
  DESCRIPTION OF ARTIFICIAL SEQUENCE  SYNTHETIC PEPTIDE
YGRKKRRQRRRGMTSSSSVV

SEQ ID NO: 170 - protein, 20 aa, Artificial Sequence
  DESCRIPTION OF ARTIFICIAL SEQUENCE  SYNTHETIC PEPTIDE
YGRKKRRQRRRARGRRETWV

SEQ ID NO: 171 - protein, 20 aa, Artificial Sequence
  DESCRIPTION OF ARTIFICIAL SEQUENCE  SYNTHETIC PEPTIDE
YGRKKRRQRRRQDERVETRV

SEQ ID NO: 172 - protein, 20 aa, Artificial Sequence
  DESCRIPTION OF ARTIFICIAL SEQUENCE  SYNTHETIC PEPTIDE
YGRKKRRQRRRLQRRRETQV

SEQ ID NO: 173 - protein, 18 aa, Artificial Sequence
  DESCRIPTION OF ARTIFICIAL SEQUENCE  SYNTHETIC PEPTIDE
SPQPDSTDNDDYDDISAA

SEQ ID NO: 174 - protein, 19 aa, Artificial Sequence
  DESCRIPTION OF ARTIFICIAL SEQUENCE  SYNTHETIC PEPTIDE
PYGTAMEKAQLKPPATSDA

SEQ ID NO: 175 - protein, 19 aa, Artificial Sequence
  DESCRIPTION OF ARTIFICIAL SEQUENCE  SYNTHETIC PEPTIDE
HKAEIHAQPSDKERLTSDA

SEQ ID NO: 176 - protein, 20 aa, Artificial Sequence
  DESCRIPTION OF ARTIFICIAL SEQUENCE  SYNTHETIC PEPTIDE
PKQANGGAYQKPTKQEEFYA

SEQ ID NO: 177 - protein, 19 aa, Artificial Sequence
  DESCRIPTION OF ARTIFICIAL SEQUENCE  SYNTHETIC PEPTIDE
AISGTSSDRGYGSPRYAEV

SEQ ID NO: 178 - protein, 20 aa, Artificial Sequence
  DESCRIPTION OF ARTIFICIAL SEQUENCE  SYNTHETIC PEPTIDE
LNSCSNRRVYKKMPSIESDV

SEQ ID NO: 179 - protein, 20 aa, Artificial Sequence
  DESCRIPTION OF ARTIFICIAL SEQUENCE  SYNTHETIC PEPTIDE
ARKANMKGSYSLVEAQKSKV

SEQ ID NO: 180 - protein, 20 aa, Artificial Sequence
  DESCRIPTION OF ARTIFICIAL SEQUENCE  SYNTHETIC PEPTIDE
ISGTPTSTMVHGMTSSSSVV

SEQ ID NO: 181 - protein, 20 aa, Artificial Sequence
  DESCRIPTION OF ARTIFICIAL SEQUENCE  SYNTHETIC PEPTIDE
KDITSDSENSNFRNEIQSLV

SEQ ID NO: 182 - protein, 19 aa, Artificial Sequence
  DESCRIPTION OF ARTIFICIAL SEQUENCE  SYNTHETIC PEPTIDE
TTNNNPNSAVNIKKIFTDV

SEQ ID NO: 183 - protein, 20 aa, Artificial Sequence
  DESCRIPTION OF ARTIFICIAL SEQUENCE  SYNTHETIC PEPTIDE
TREDIYVNYPTFSRRPKTRV

SEQ ID NO: 184 - protein, 19 aa, Artificial Sequence
  DESCRIPTION OF ARTIFICIAL SEQUENCE  SYNTHETIC PEPTIDE
SSAKSSNKNKKNKDKEYYV

SEQ ID NO: 185 - protein, 19 aa, Artificial Sequence
  DESCRIPTION OF ARTIFICIAL SEQUENCE  SYNTHETIC PEPTIDE
TSGTGHNQTRALRASESGI

SEQ ID NO: 186 - protein, 19 aa, Artificial Sequence
  DESCRIPTION OF ARTIFICIAL SEQUENCE  SYNTHETIC PEPTIDE
QGDPALQDAGDSSRKEYFI

SEQ ID NO: 187 - protein, 20 aa, Artificial Sequence
  DESCRIPTION OF ARTIFICIAL SEQUENCE  SYNTHETIC PEPTIDE
SVFSIPTLWSPWPPSSSSQL

SEQ ID NO: 188 - protein, 19 aa, Artificial Sequence
  DESCRIPTION OF ARTIFICIAL SEQUENCE  SYNTHETIC PEPTIDE
KDSRPSFVGSSSGHTSTTL

SEQ ID NO: 189 - protein, 20 aa, Artificial Sequence
  DESCRIPTION OF ARTIFICIAL SEQUENCE  SYNTHETIC PEPTIDE
HDFRRAFKKILARGDRKRIV

SEQ ID NO: 190 - protein, 20 aa, Artificial Sequence
  DESCRIPTION OF ARTIFICIAL SEQUENCE  SYNTHETIC PEPTIDE
STDNLWRPFMDTLASHQLYI